(19) United States
(12) Patent Application Publication
(10) Pub. No.: US 2022/0002699 A1
(43) Pub. Date: Jan. 6, 2022

(54) ACE2-Fc FUSION PROTEINS FOR SARS-COV-2 MITIGATION

(71) Applicant: PLANET BIOTECHNOLOGY, INC., HAYWARD, CA (US)

(72) Inventors: Keith Lynn WYCOFF, Palo Alto, CA (US); Y TRAN, HAYWARD, CA (US)

(73) Assignee: PLANET BIOTECHNOLOGY, INC., HAYWARD, CA (US)

(21) Appl. No.: 17/194,282

(22) Filed: Mar. 7, 2021

Related U.S. Application Data
(60) Provisional application No. 63/100,321, filed on Mar. 7, 2020.
(60) Provisional application No. 63/100,503, filed on Mar. 12, 2020.
(60) Provisional application No. 63/100,549, filed on Mar. 16, 2020.
(60) Provisional application No. 63/100,697, filed on Mar. 25, 2020.
(60) Provisional application No. 63/100,812, filed on Apr. 1, 2020.
(60) Provisional application No. 63/100,838, filed on Apr. 3, 2020.
(60) Provisional application No. 63/101,086, filed on Apr. 14, 2020.
(60) Provisional application No. 63/101,400, filed on Apr. 27, 2020.
(60) Provisional application No. 63/101,594, filed on May 5, 2020.
(60) Provisional application No. 63/102,161, filed on May 28, 2020.
(60) Provisional application No. 63/103,890, filed on Aug. 28, 2020.
(60) Provisional application No. 63/204,960, filed on Nov. 3, 2020.

(51) Int. Cl. C12N 9/48; C07K 14/165

(52) U.S. Cl. CPC C12N 9/485; C07K 14/165; A61K 38/00; C07K 2319/30; C07K 2319/91; C12Y 304/17023

(57) ABSTRACT

The present disclosure relates to recombinant fusion proteins comprising an extracellular domain of the human angiotensin-converting enzyme 2 (ACE2), optionally having altered amino acid residues that result in increased binding affinity for the S1 spike protein of SARS-CoV-2, linked to a human immunoglobulin Fc region, that can extend the protein half-life (T_{1/2}) and/or the duration of action as a decoy receptor, and compositions and methods of use of these fusion proteins.

### CROSS REFERENCE TO RELATED APPLICATIONS

This application claims priority of US Provisional Patent Application Nos. 63/100,321, filed Mar. 7, 2020, 63/100,503, filed Mar. 12, 2020, No. 63/100,549, filed Mar. 16, 2020, 63/100,697, filed Mar. 25, 2020, 63/100,812, filed Apr. 1, 2020, 63/100,838, filed Apr. 3, 2020, 63/101,086, filed Apr. 14, 2020, 63/101,400, filed Apr. 27, 2020, 63/101,594, filed May 5, 2020, 63/102,161, filed May 28, 2020, 63/103,890, filed Aug. 28, 2020, and 63/204,960, filed Nov. 3, 2020, each of which is hereby incorporated by reference herein.

### REFERENCE TO SEQUENCE LISTING

The official copy of the Sequence Listing is submitted as an ASCII formatted text file with a file name of “16188-001WO1_SeqList_ST25.txt”, a creation date of May 17, 2021, and a size of 174,071 bytes. The Sequence Listing is part of the specification and is incorporated in its entirety by reference herein.

### FIELD

The present disclosure relates to recombinant fusion proteins comprising an extracellular domain of the human angiotensin-converting enzyme 2 (ACE2), optionally having altered amino acid residues that result in increased binding affinity for the S1 spike protein of SARS-CoV-2, linked to a human immunoglobulin Fc region, that can extend the protein half-life (T_{1/2}) and/or the duration of action as a decoy receptor, and compositions and methods of use of these fusion proteins.

### BACKGROUND

A novel coronavirus, SARS-CoV-2, was first identified in humans in 2019 in China and has rapidly spread world-wide to over 70 countries by March 2020. Preliminary epidemiology studies indicate human-to-human transmission of this deadly virus, leading to global concern about a COVID-19 pandemic. Genetic and phylogenetic characterization shows that SARS-CoV-2 belongs to lineage B of the betacoronavirus genus. The direct source and reservoirs of SARS-CoV-2 remain enigmatic. Certain amino-acid homology between the SARS-CoV-2 infecting humans and coronaviruses isolated from pangolins and turtles suggest a zoonotic origin of this emerging pathogen.

A developing understanding of SARS-CoV-2 biology has proceeded rapidly, leading to numerous possibilities for therapeutic and vaccine development. Like other coronaviruses, the SARS-CoV-2 virion uses a large surface spike (S) glycoprotein for interaction with and entry into target cells. The S glycoprotein consists of a globular S1 domain at its N-terminus, followed by a membrane-proximal S2 domain, a transmembrane domain and an intracellular domain at its C-terminus. Determinants for cellular tropism and interaction with the target cell are within the S1 domain, while mediators of membrane fusion are within the S2 domain.

Angiotensin convening enzyme 2 (ACE2) is an exopeptidase that catalyzes the conversion of angiotensin I to the nonapeptide angiotensin 1-9 (Donoghue et al. 2000), or the conversion of angiotensin II to angiotensin 1-7 (Keidar, Kaplan, and Gamliel-Lazarovich 2007; Wang et al. 2016). ACE2 has direct effects on cardiac function and is expressed predominantly in vascular endothelial cells of the heart and the kidneys (Boehm and Nabel 2002). ACE2 receptors have been shown to be the entry point into human cells for some coronaviruses, including the SARS-CoV virus (Kuba et al. 2005). A number of studies have identified that the entry point is the same for SARS-CoV-2, the virus that causes COVID-19 (Zhou et al. 2020).

The structure of the S1 glycoprotein of SARS-CoV-2 bound to ACE2 was solved by Liu et al. (Liu et al. 2020). The structure of the SARS-CoV-2 S1 RBD:human ACE2 complex also has been resolved (PDB: 2AJF). Recently, the structure of SARS-CoV-2 RBD bound to human ACE2 has been worked out as well (PDB: 6LZG and 6M0J). The overall structure of the ACE2: RBD surface interfaces are similar with the two viruses. However, the affinity of human ACE2 for SARS-CoV-2 S1 RBD (˜15 nM) is 10-20 times higher than its affinity for SARS-CoV S1 RBD, which may explain the higher infectivity of SARS-CoV-2.

Some form of soluble (not membrane-bound) ACE2 has been suggested as a potential therapeutic for COVID-19, based on in vitro experiments with SARS-CoV. Recombinant soluble ACE2 blocked the attachment of SARS-CoV S protein to African Green Monkey cells and blocked SARS-CoV infection of human embryonic kidney cells in a dose-dependent manner. Apeiron Biologics has initiated a Phase 2 clinical trial of APN01, a recombinant human ACE2, in COVID-19 patients, on the hypothesis that rhACE2 will bind to the virus, preventing it from binding to and infecting cells. Prior trials had found intravenous administration of rhACE2 (developed as a treatment for pulmonary arterial hypertension and acute lung injury) to be safe and well tolerated at up to 0.4 mg/kg twice a day (BID). Another trial of rhACE2 against COVID-19 had been initiated in China, but has since been withdrawn.

An anti-ACE2 antibody has been proposed as a potential therapeutic to block infection in vitro by SARS-CoV-2, but there are potential problems with this approach. Blocking a widespread human cell-surface antigen with an antibody may have pleiotropic effects on the host or patient. Such an antibody may stimulate a receptor response upon binding or may interfere with or prevent binding of a normal ligand to the receptor. In addition, to the extent that ACE2 circulates in the serum of infected individuals and could be bound by an anti-ACE2 antibody, a greater parenteral dose of soluble ACE2 may be required to achieve a therapeutic effect. Furthermore, an unknown quantity of ACE2 is found on cell surfaces, so, a significant amount of antibody may be needed to block enough cell surface SARS-COV-2 binding sites to prevent infection.

An alternative approach is to provide an antibody against the S1 domain on the SARS-CoV-2 spike protein. In fact, S-protein-specific neutralizing antibodies were generated in patients recovering from SARS. Unfortunately, the development of escape mutants of SARS-CoV-2 that mutate to carry S1 domain-proteins that bind to the antibody yet are still able to bind to the ACE2 receptor, can occur, as has been seen previously with anti-S mAbs to SARS-CoV (Rockx et al. 2010).

### SUMMARY

Generally, the present disclosure provides recombinant fusion proteins comprising the human ACE2 protein, which acts as the cell surface receptor for binding to the SARS-CoV-2 receptor binding domain (RBD), that can disrupt the initial steps binding involved of in SARS-CoV-2 infection. In at least one embodiment, the recombinant fusion protein comprises an extracellular domain of the human ACE2 protein, optionally with amino acid changes that improve binding affinity to SARS-CoV-2 spike protein, fused to a Fc region of a human immunoglobulin, which extends the fusion protein half-life (T_{1/2}). The ACE2-Fc fusion proteins of the present disclosure are capable of acting as a “receptor decoy” to prevent the interaction of SARS-CoV-2 S1 spike protein with its receptor, the human ACE2 protein on human cells, thereby stopping infection. Furthermore, the ACE2-Fc fusion protein's ability to act as a receptor decoy does not allow the virus to select for neutralization escape mutants, as any mutation of the viral spike protein that decreases binding to the receptor decoy fusion protein also decreases virus binding affinity for the native huACE2 receptor, resulting in an attenuated virus.

This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides a modified human ACE2 protein comprising at least amino acids 19-614 of the human ACE2 protein sequence of SEQ ID NO: 1 with at least one consensus contact sequence residue altered relative to SEQ ID NO: 1, wherein affinity of the modified human ACE2 protein for the S1 spike protein of SARS-CoV-2 is increased relative to affinity of the human ACE2 protein of SEQ ID NO: 1 for the S1 spike protein of SARS CoV-2.

In at least one embodiment of the modified human ACE2 protein, said at least one altered residue is selected from amino acid residues 30-42, 81-84, 327-329, and/or 353-357. In at least one embodiment, said at least one altered residue is selected from amino acid residues 30, 31, 34, 38, 40, 41, 81, 82, 329, and/or 354. In at least one embodiment, said at least one altered residue comprises an amino acid change selected from D30E, D30S, K31Q, K31E, H34S, H34V, D38E, F40S, Q42A, Q81K, M82N, M82K, M82T, E329N, E329K, G354H, G354K, and combinations thereof. In at least one embodiment, said at least one residue altered comprises at least two amino acid changes, wherein the two changes are selected from: D38E and F42S, and/or Q81K and M82N. In at least one embodiment, said at least one residue altered is an amino acid change selected from H34S and H34V

In at least one embodiment of the modified human ACE2 protein, at least one N-glycosylation site residue of the ACE2 protein is changed from an N to an amino acid residue that does not glycosylate; optionally, wherein the at least one N-glycosylation site is changed from an N to S. In at least one embodiment, the N-glycosylation site residue at position 546 has been changed from N to S.

In at least one embodiment of the modified human ACE2 protein, an amino acid residue adjacent to an N-glycosylation site residue is changed to an amino acid residue that prevents glycosylation at the adjacent N-glycosylation site. In at least one embodiment, the amino acid residue at position 547 adjacent to the N-glycosylation site at position 546 is changed from S to P.

In at least one embodiment of the modified human ACE2 protein, the ACE2 protein comprises a fusion with a human immunoglobulin Fc region. In at least one embodiment, said human immunoglobulin is selected from the group consisting of IgG1, IgG2, IgG3, IgG4, IgA1, IgA2, IgM, IgD, and IgE. In at least one embodiment, the human immunoglobulin Fc region comprises an amino acid sequence of any one of SEQ ID NOs: 18-22.

In at least one embodiment of the modified human ACE2 protein comprising a fusion with a human immunoglobulin Fc region, said Fc region amino terminus is linked to said ACE2 protein carboxy terminus. In at least one embodiment, said Fc region carboxy terminus linked to said ACE2 protein amino terminus. In at least one embodiment, the ACE2 protein fusion with the Fc region comprises an amino acid sequence selected from any one of SEQ ID NO: 4, 5, 6, 7, 8, 9, 10, 11, 12, 13, 14, 15, 16, and 17.

In at least one embodiment of the modified human ACE2 protein comprising a fusion with a human immunoglobulin Fc region, said fusion further comprises a linker; optionally, wherein the linker comprises an amino acid sequence of any one of SEQ ID NOs: 23-49.

In at least one embodiment of the modified human ACE2 protein comprising a fusion with a human immunoglobulin Fc region, said Fc region further comprises a KDEL sequence at the carboxy terminus thereof.

In at least one embodiment of the modified human ACE2 protein comprising a fusion with a human immunoglobulin Fc region, the protein has typical mammalian glycosylation. In at least one embodiment, the N-glycans of said protein lack detectable β1,2-xylose and α1,3-fucose residues. In at least one embodiment, the protein comprises a terminal β1,4-Gal residue at an N-glycan.

In at least one embodiment of the modified human ACE2 protein comprising a fusion with a human immunoglobulin Fc region, the protein is produced in a DXT/FT plant; optionally, wherein the plant is N. benthamiana. In at least one embodiment, the DXT/FT plant is modified to add terminal β1,4-Gal residues to N-glycan protein; optionally, wherein the modification comprises prior infiltration or co-infiltration with a binary vector encoding a human β1,4-galactosyl-transferase (ST-GalT). In at least one embodiment, the ACE2 protein is humanized.

In at least one embodiment of the modified human ACE2 protein, the protein is enzymatically active. In at least one embodiment, the protein is enzymatically inactive; optionally, wherein the protein amino acid sequence comprises the amino acid change R273K or R273Q, relative to SEQ ID NO: 1.

In at least one embodiment the present disclosure provides a chimeric SARS-CoV-2 S1 spike protein receptor comprising: an immunoglobulin complex, wherein the immunoglobulin complex comprises at least a portion of an immunoglobulin heavy chain, and a modified ACE2 protein linked to the immunoglobulin heavy chain, wherein the modified ACE2 protein comprises at least amino acids 19-614 of the human ACE2 protein sequence of SEQ ID NO: 1 with at least one consensus contact sequence residue altered relative to SEQ ID NO: 1 to increase affinity for the SARS-Cov-2 S1 spike protein.

In at least one embodiment of the chimeric SARS-CoV-2 S1 spike protein receptor, the immunoglobulin complex further comprises at least a portion of an immunoglobulin light chain; optionally, wherein portion comprises a kappa chain or a lambda chain. In at least one embodiment, the linkage between the modified ACE2 protein and the immunoglobulin heavy chain comprises an immunoglobulin hinge.

In at least one embodiment of the chimeric SARS-CoV-2 S1 spike protein receptor, the immunoglobulin heavy chain is from an immunoglobulin selected from the group consisting of IgG1, IgG2, IgG3, IgG4, IgA1, IgA2, IgM, IgD, and IgE. In at least one embodiment, the human immunoglobulin heavy chain is an Fc region fragment; optionally wherein the fragment comprises an amino acid sequence of any one of SEQ ID NOs: 18-22. In at least one embodiment, the immunoglobulin heavy chain is from an IgG and comprises heavy chain constant regions 2 and 3 thereof. In at least one embodiment, the immunoglobulin heavy chain is from an IgA and comprises heavy chain constant regions 2 and 3 thereof.

In at least one embodiment, the present disclosure also provides a nucleic acid comprising a sequence encoding a modified human ACE2 protein or a chimeric SARS-CoV-2 S1 spike protein receptor as disclosed herein. In at least one embodiment, the present disclosure also provides an expression vector comprising such a nucleic acid encoding a modified human ACE2 protein or a chimeric SARS-CoV-2 S1 spike protein receptor as disclosed herein.

In at least one embodiment, the present disclosure also provides a composition comprising the chimeric SARS-CoV-2 S1 spike protein receptor as disclosed herein and plant material. In at least one embodiment, the plant material is selected from the group consisting of: plant cell walls, plant organelles, plant cytoplasm, intact plant cells, plant seeds, and viable plants.

In at least one embodiment, the present disclosure also provides a method for reducing binding of SARS-CoV-2 to a host cell, the method comprising: contacting the SARS-CoV-2 with the chimeric SARS-CoV-2 S1 spike protein receptor of any one of claims 32-38, wherein the chimeric receptor binds to the SARS-CoV-2 Receptor Binding Domain (RBD) thereby reducing the binding of SARS-CoV-2 to the host cell.

In at least one embodiment, the present disclosure provides a method for producing a chimeric SARS-CoV-2 S1 spike protein receptor as disclosed herein, wherein the method comprises introducing an expression vector comprising a nucleic acid encoding the chimeric SARS-CoV-2 S1 spike protein receptor into a cellular host and expressing the immunoglobulin complex and the ACE2 peptide to form the chimeric SARS-CoV-2 S1 spike protein receptor. In at least one embodiment, the host is a plant.

In at least one embodiment, the present disclosure also provides a method for producing a modified human ACE2 protein of as disclosed herein, the method comprising introducing an expression vector comprising a nucleic acid encoding the ACE2 protein into a cellular host; and expressing the ACE2 protein. In at least one embodiment, the host is a plant.

In at least one embodiment, the present disclosure also provides a pharmaceutical composition comprising a modified human ACE2 protein or a chimeric SARS-CoV-2 S1 spike protein receptor as disclosed herein and a pharmaceutically acceptable carrier.

In at least one embodiment, the present disclosure also provides a method for reducing binding of SARS-CoV-2 to a cell, the method comprising: contacting the SARS-CoV-2 with the modified human ACE2 protein or a chimeric SARS-CoV-2 S1 spike protein receptor as disclosed herein, wherein the modified human ACE2 protein binds to the SARS-CoV-2 Receptor Binding Domain (RBD), thereby reducing the binding of SARS-CoV-2 to the cell.

In at least one embodiment, the present disclosure also provides provides a modified human ACE2 protein or a chimeric SARS-CoV-2 S1 spike protein receptor as disclosed herein for use in treating or preventing a SARS-CoV-2 infection, or the effects thereof.

In at least one embodiment, the present disclosure also provides a modified human ACE2 protein or a chimeric SARS-CoV-2 S1 spike protein receptor as disclosed herein for use as a medicament, or for use in the preparation of a medicament.

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1A shows the full-length amino acid sequence of human Angiotensin converting enzyme 2 (ACE2) (residues 1-805; SEQ ID NO: 1), with consensus contact sequences, catalytic domain and sites of N-glycan attachment indicated in green, red, and orange, respectively.

FIG. 1B shows the extracellular domain of human ACE2, also referred to herein as the soluble ACE2 receptor, comprising amino acid sequence of SEQ ID NO: 2, which sequence corresponds residues 19-614 of SEQ ID NO: 1.

FIG. 1C shows the 2166 nucleotide sequence of SEQ ID NO: 3 which encodes the open reading frame of the full length soluble domain of the human Angiotensin converting enzyme 2 (ACE2) amino acid sequence corresponding to residues 19-740 of SEQ ID NO: 1. Nucleotides 1-1788 of SEQ ID NO: 3 encode residues 19-614 of SEQ ID NO: 1.

FIG. 2A shows the amino acid sequence of the huACE2-Fc (IgG₁) fusion protein of SEQ ID NO: 4, which corresponds to the C-terminus of the soluble extracellular domain of amino acids 19-614 of the human ACE2 sequence of SEQ ID NO: 1 fused in sequence to the N-terminus of the Fc region of IgG₁.

FIG. 2B shows the amino acid sequence of the Modified ACE2(H34S)-Fc (IgG₁) fusion protein of SEQ ID NO: 5, which corresponds to the C-terminus of the soluble extracellular domain of amino acids 19-614 of the human ACE2 sequence of SEQ ID NO: 1 with an H34S amino acid change fused in sequence to the N-terminus of the Fc region of IgG₁.

FIG. 3 shows the amino acid sequence of huACE2-Fc (IgA1) fusion protein of SEQ ID NO: 6, which corresponds to the C-terminus of the soluble extracellular domain of amino acids 19-614 of the human ACE2 sequence of SEQ ID NO: 1 fused in sequence to the N-terminus of the Fc region of IgA₁.

FIG. 4 shows the amino acid sequence of huACE2-Fc (IgA2) fusion protein of SEQ ID NO: 7, which corresponds to the C-terminus of the soluble extracellular domain of amino acids 19-614 of the human ACE2 sequence of SEQ ID NO: 1 fused in sequence to the N-terminus of the Fc region of IgA2.

FIG. 5 shows the amino acid sequence of (IgG₁) Fc-huACE2 fusion protein of SEQ ID NO: 8, which corresponds to the C-terminus of the IgG₁ Fc region fused in sequence to the N-terminus of the soluble extracellular domain of amino acids 19-614 of the human ACE2 sequence of SEQ ID NO: 1 (i.e., in the reverse orientation of the fusion protein of FIG. 2A).

FIG. 6 shows the amino acid sequence of (IgA1) Fc-huACE2 fusion protein of SEQ ID NO: 9, which corresponds to the C-terminus of the IgA1 Fc region fused in sequence to the N-terminus of the soluble extracellular domain of amino acids 19-614 of the human ACE2 sequence of SEQ ID NO: 1.

FIG. 7 shows the amino acid sequence of (IgA2) Fc-huACE2 fusion protein of SEQ ID NO: 10, which corresponds to the C-terminus of the IgA2 Fc region fused in sequence to the N-terminus of the soluble extracellular domain of amino acids 19-615 of the human ACE2 sequence of SEQ ID NO: 1.

FIG. 8 shows the amino acid sequence of huACE2-Fc (IgG₃) fusion protein of SEQ ID NO: 11, which corresponds to the C-terminus of the soluble extracellular domain of amino acids 19-614 of the human ACE2 sequence of SEQ ID NO: 1 fused in sequence to the N-terminus of the Fc region of IgG₃.

FIG. 9 shows the amino acid sequence of (IgG₃) Fc-huACE2 fusion protein of SEQ ID NO: 12, which corresponds to the C-terminus of the IgG₃ Fc region fused in sequence to the N-terminus of the soluble extracellular domain of amino acids 19-614 of the human ACE2 sequence of SEQ ID NO: 1.

FIG. 10 shows the amino acid sequence of huACE2-Fc (IgG₁) complement activation knockout fusion protein of SEQ ID NO: 13, which corresponds to the C-terminus of the soluble extracellular domain of amino acids 19-614 of the human ACE2 sequence of SEQ ID NO: 1 fused in sequence to the N-terminus of the Fc region of IgG₁, in which the IgG₁ immunoglobulin residues D652 and K704 are changed to A652 and A704.

FIG. 11 shows the amino acid sequence of (IgG₁) complement activation knockout Fc-huACE2 fusion protein of SEQ ID NO: 14, which corresponds to the to the C-terminus of the IgG₁ Fc region fused in sequence to the N-terminus of the soluble extracellular domain of amino acids 19-614 of the human ACE2 sequence of SEQ ID NO: 1, in which the IgG₁ immunoglobulin residues D55 and K107 are changed to A55 and A107 (i.e., in the reverse orientation of the fusion protein of FIG. 10).

FIG. 12 shows plasmid maps of the plant expression vectors used to express ACE-2-Fc The ACE2-Fc sequence is depicted as integrated into the plasmid pTRAk which has the following regions: P35SS, the CaMV 35S promoter with duplicated transcriptional enhancer; CHS, chalcone synthase 5′ untranslated region; pA35S, CaMV 35S polyadenylation signal; SAR, scaffold attachment region of the tobacco Rb7 gene; LB and RB, the left and right borders for T-DNA integration; ColE1ori, origin of replication for E. coli; RK2ori, origin of replication for Agrobacterium; b/a, ampicillin/carbenicillin resistance gene; LPH, signal peptide sequence from the murine mAb24 heavy chain; npt II, kanamycin resistance gene; Pnos and pAnos, promoter and polyadenylation signal of the nopaline synthase gene. pTRA-P19 is a plasmid that may be co-infiltrated with pTrak-ACE2-Fc. This plasmid provides the silencing supressor sequence P19. The regions named in pTRA-P19 have the same meaning as described in plasmid pTRAk

FIG. 13A shows the amino acid sequence of huACE2-Fc (IgG4) fusion protein of SEQ ID: 15 which corresponds to the C-terminus of the soluble extracellular domain of amino acids 19-614 of the human ACE2 sequence of SEQ ID NO: 1 fused in sequence to the N-terminus of the Fc region of IgG₄.

FIG. 13B shows the amino acid sequence of Modified ACE2-Fc (IgG₄) which corresponds to the C-terminus of the soluble extracellular domain of amino acids 19-614 of the human ACE2 sequence of SEQ ID NO: 1 with an H34S amino acid change fused in sequence to the N-terminus of the Fc region of Iga₄.

FIG. 14 shows the amino acid sequence of (IgG4) Fc-huACE2 which corresponds to the C-terminus of the IgG4 Fc region fused in sequence to the N-terminus of the soluble extracellular domain of amino acids 19-614 of the human ACE2 sequence of SEQ ID NO: 1 (i.e., in the reverse orientation of the fusion protein of FIG. 13A).

FIG. 15 shows results of SDS-PAGE analysis of ACE2-Fc fusion proteins. The gel images show that structural integrity of four ACE2-Fc fusion proteins in reducing and non-reducing SDS-PAGE gels and immunoblots of the same ACE2-Vc variant proteins with Fc-specific antibodies (Southern Biotechnology) and ACE2-specific antibodies (R & D Systems). Samples migrated at ˜250 kD under non-reducing conditions and signal at ˜250 kD for both anti-huIgG and anti-ACE2 immunoblots indicated that the purified samples contain both ACE2 and Fc as expected.

FIG. 16 depicts a graph of results of binding affinity study of huACE2-Fc (IgG₁) fusion protein of SEQ ID NO: 4 and the modified ACE2(H34S)-Fc (IgG₁) fusion protein of SEQ ID NO: 5. The results show that the modified ACE2(H34S)-Fc (IgG₁) fusion protein, which includes the amino acid change H34S, exhibits improved affinity for binding SARS-CoV-2 S1 spike protein

FIG. 17 shows a map of plasmid p1449, pTRAk-c-lph-ACE2(273K)-hFc1.

FIG. 18 shows a map of plasmid p1247, pTRAk-c-lph-oDPP4(39-766, mV1)-hFc(hr4.1).

FIG. 19 shows a map of plasmid p1473, pTRAk-c-lph-ACE2 (19-614)-hFc1.

FIG. 20 shows a map of plasmid p1461, pTRAk-c-lph-ACE2 (19-614) (34S,273K)-hFc1.

FIG. 21 depicts a plot showing the improved binding affinity of ACE2-Fc fusion proteins of the present disclosure for the SARS-CoV2 S1 spike protein.

FIG. 22A and FIG. 22B depict images of SDS-PAGE analysis of samples of ACE2-Fc fusions expressed in N. benthamiana wild type or DXT/FT plants as described in Example 10.

FIGS. 23A and 23B depict plots of ELISA binding assays of samples of ACE2-Fc fusions expressed in N. benthamiana wild type or DXT/FT plants as described in Example 10.

FIG. 24 depicts a plot of results showing effects of H34S mutation on ACE2-Fc binding to SARS-CoV-2 Spike S1 protein as described in Example 11.

FIG. 25 depicts plots of carboxypeptidase activity assay results for various ACE2-Fc fusion proteins as described in Example 12.

FIG. 26A and FIG. 26B depicts HPLC traces of nebulized ACE2-Fc fusion protein samples prepared as described in Example 14.

FIG. 27 depicts plots of results of binding assays of nebulized ACE2-Fc fusion proteins as described in Example 14.

FIG. 28 depicts plots of enzymatic activity assay results of nebulized ACE2-Fc fusion proteins as described in Example 14.

FIG. 29A, FIG. 29B, FIG. 29C, FIG. 29D, and FIG. 29E depict plots of assay results of ACE2-Fc fusion protein binding to the S1 Spike Protein from SARS-CoV-2 variants, Wuhan, UK, Mink, South Africa and UK Plus, as described in Example 15.

### DETAILED DESCRIPTION

A novel coronavirus, recently designated SARS-CoV-2, causes the respiratory disease COVID-19, a newly emerging human health threat with a more than 2% case fatality rate. The cell surface protein angiotensin-converting enzyme 2 (ACE2) is used by SARS-CoV-2 to enter and infect cells. Soluble ACE2 binds the SARS-CoV-2 spike (S) glycoprotein and inhibits SARS-CoV-2 infection of VERO cells. The present disclosure describes a superior inhibitor of SARS-CoV-2 infection comprising a fusion of a modified ACE2 binding sequence to the Fc of human immunoglobulin. This construct exhibits a potency greater than that expected, due to the stoichiometry of ACE2 in the Fc fusion (two ACE2 binding domains per molecule). In addition, to the improved potency, the modified ACE2-Fc fusion protein should have a long circulating half-life and the ability to be delivered to airway mucosal surfaces, the primary site of SARS-CoV-2 infection. Furthermore, unlike anti-SARS-CoV-2 antibodies which have been proposed as potential therapeutics, the modified ACE2-Fc fusion proteins of the present disclosure can act as “receptor decoys” for SARS-CoV-2 binding, and will not subject the virus to selection for neutralization escape mutants, as any mutation that decreases binding to the receptor decoy will necessarily decrease binding to the native receptor, resulting in an attenuated virus.

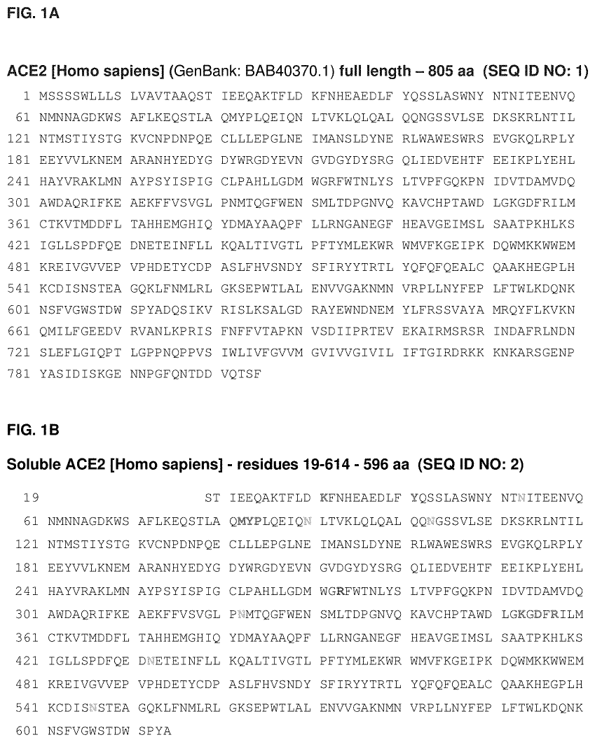

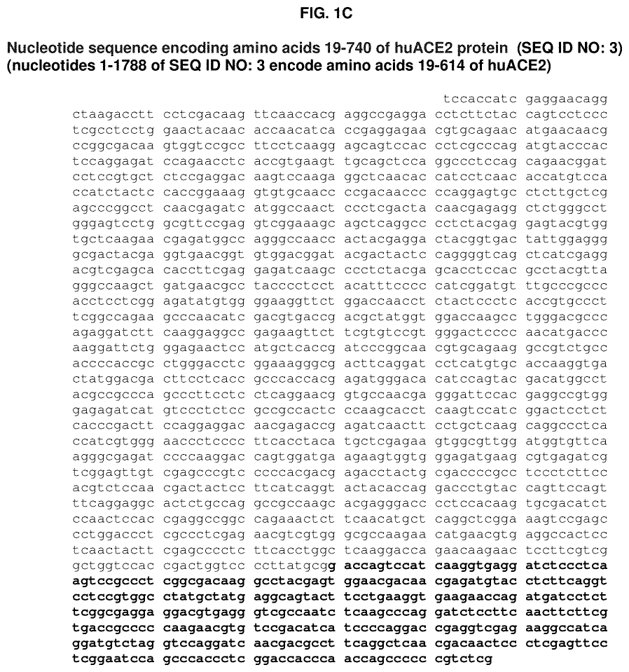

As described in greater detail elsewhere herein, the anti-SARS-CoV-2 inhibitory potency of the modified human ACE2 protein fused to the Fc of the various different human immunoglobulin isotypes—IgG1, IgG3, IgG4, IgA1 and IgA2—is increased compared to the same Fc fusions of unmodified ACE2. Fusions of Fc and the full-length ACE2 extracellular domain (describe variously in the literature as amino acids 19-615 and 19-740 of the 805 amino acid long sequence shown in FIG. 1) are described, and genetic constructs capable of expression by eukaryotic host cells, organs or organisms are provided. In a preferred, but not limiting embodiment, the modified ACE2-Fc fusion is expressed using a rapid transient plant expression system. Purified modified ACE2-Fc fusions and formulations thereof are also shown. The ability of the ACE2-Fc variants to bind the S1 domain of the SARS-CoV-2 spike protein in a functional ELISA as well as in cell culture is disclosed. In further embodiments, of the modified ACE2-Fc fusion, single or multiple amino acid changes at specific regions in the human ACE2 are disclosed that further improve binding to the SARS-CoV-2 spike protein and SARS-Cov-2 viral inactivation in vitro and in vivo.

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00-130, (1987-2020), published by Wiley & Sons, Inc. in the Wiley Online Library (hereinafter “Ausubel”); M. J. MacPherson, et al., eds. Pcr 2: A Practical Approach (1995); Harlow and Lane, eds, Antibodies: A Laboratory Manual (1988), and H. Jones, Methods In Molecular Biology vol. 49, “Plant Gene Transfer And Expression Protocols” (1995)

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

Immunoadhesin: A complex containing a chimeric receptor protein molecule, and optionally containing a secretory component and J chain.

Chimeric receptor protein: A receptor-based protein having at least a portion of its amino acid sequence derived from an extracellular receptor and at least a portion derived from an immunoglobulin complex.

Receptor: As used herein, the term refers to any polypeptide that binds to specific antigens as defined herein, or any proteins, lipoproteins, glycoproteins, polysaccharides or lipopolysaccharides that exert or lead to exertion of a biological or pathogenic effect with an affinity and avidity sufficient to allow a chimeric receptor protein to act as a receptor decoy. For example, a receptor may be a viral attachment receptor such as ICAM-1, which is a receptor for human rhinovirus, or DPP4 which is a receptor for MERS-CoV spike glycoprotein 1, or ACE2 which is a receptor for SARS 2 spike glycoprotein 1, or a receptor for a bacterial toxin, such as CMG2 which is one of the receptors for anthrax protective antigen, or tumor necrosis factor receptor superfamily (TNFRSF) is a group of cytokine receptors characterized by the ability to bind tumor necrosis factors (TNFs) via an extracellular cysteine-rich domain. The receptors as used herein shall at a minimum contain the functional elements for binding of a component or components of the molecule to which they bind but may optionally also include one or more additional polypeptides.

Functional ACE2 means an amino acid sequence substantially identical to SEQ ID NO: 1 (shown in FIG. 1) that maintains the ability to bind to the SARS-CoV-2 S1 glycoprotein. Such functional ACE2 amino acid sequences can comprise the entire human ACE2 protein sequence (805 amino acids) of SEQ ID NO: 1 portions of the human ACE2 polypeptide of SEQ ID NO: 1, including the extracellular domain or soluble domain portions that consist of residues 19-740, or a shorter sequence of the extra cellular domain of 597 amino acid residues spanning residues 19-615, or an even a smaller sub-region of soluble receptor of the ACE2 protein required for binding to SARS-CoV-2 (fewer than 597 amino acid residues) such as 19-614 of SEQ ID NO: 1. A functional ACE2 also may include a Modified ACE2 as herein defined below. Such a Modified ACE2 alone, or associated with a Moiety, for example a fusion with an Fc region, may also be referred to herein as variants.

Modified ACE2 sequence means, with reference the amino acid sequence of human ACE2 protein (SEQ ID NO: 1, as shown in FIG. 1), an ACE2 sequence in which one or more amino acids in the primary sequence of SEQ ID NO: 1 has be changed to another amino acid or has been deleted. Using the amino acid numbering SEQ ID NO: 1, such modified ACE 2 sequence may have such amino acid changes in one or more than one of four general regions on ACE2 that have been identified to be important for binding SARS coronavirus S glycoprotein: (i) residues comprising ACE2 α-helix 1; (ii) residues comprising the ACE2 loop 2; (iii) residues comprising the ACE2 β-sheet 5 (Han, Penn-Nicholson, and Cho 2000: and residues comprising ACE2 α-helix 10. Modification of specific amino acid residues within these three regions are expected to achieve increased binding affinity of the ACE2 receptor for the SARS spike protein.

Consensus contact sequence of ACE2: those amino acid residues found in three general regions of functional ACE2 identified to be important for binding SARS-CoV-2 S1 glycoprotein: with reference to SEQ ID NO: 1 (shown in FIG. 1) (i) residues found in the amino acid sequence spanning residues 31 to 41 on α-helix 1 of ACE2; (ii) residues found in the amino acid sequence spanning residues 82, to 84 on loop 2 of ACE2; and (iii) residues found in the amino acid sequence spanning residues 353 to 357 on β-sheet 5 of ACE (Han, Penn-Nicholson, and Cho 2006).

SARS-CoV-2 Receptor Binding Domain (RBD): A sequence of amino acid residues of SARS-CoV-2 S1 spike protein containing within this sequence the regions that contact amino acid residues located in ACE2. The receptor binding domain of SARS-CoV-2 maps spans from amino acids 329-521 in the spike protein that efficiently elicits neutralizing antibodies (Liu et al. 2020).

Chimeric SARS-CoV-2 spike glycoprotein 1 receptor protein: A protein having at least a portion of its amino acid sequence derived from a functional ACE2 receptor and at least a portion derived from an immunoglobulin complex. The immunoglobulin complex may contain only a portion of an immunoglobulin heavy chain or it may contain both a portion of a heavy chain and a portion of a light chain.

Immunoglobulin molecule or antibody: A polypeptide or multimeric protein containing the immunologically active portions of an immunoglobulin heavy chain and immunoglobulin light chain covalently coupled together and capable of specifically combining with antigen. The immunoglobulins or antibody molecules are a large family of molecules that include several types of molecules such as IgM, IgD, IgG, IgA, secretory IgA (SIgA), and IgE.

Immunoglobulin complex: A polypeptide complex that can include a portion of an immunoglobulin heavy chain or both a portion of an immunoglobulin heavy chain and an immunoglobulin light chain. The two components can be associated with each other via a variety of different means, including covalent linkages such as disulfide bonds. Examples of an immunoglobulin complex include FaB′ and FaB′2.

Portion of an Immunoglobulin heavy chain: s used herein, the term refers to that region of a heavy chain which is necessary for conferring at least one of the following properties on the chimeric receptor proteins as described herein: ability to multimerize, effector functions such as binding to Fc receptors, neonatal Fc receptors or compliment fixation, proteins, ability to be purified by Protein G or A, or improved pharmacokinetics. Typically, this includes at least a portion of the heavy chain constant region. Exemplary portions of an immunoglobulin heavy chain useful in the fusion proteins of the present disclosure include the Fc region portions IgG1, IgG4, IgG3, IgA1, and IgA2, comprising an amino acid sequence of any one of SEQ ID NOs: 18-22.

Fc region: The C-terminal portion of an immunoglobulin heavy chain that interacts with cell surface receptors called Fc receptors and some proteins of the complement system. This property allows antibodies to activate the immune system. In IgG, IgA and IgD antibody isotypes, the Fc region is composed of two identical protein fragments, derived from the second and third constant domains of the antibody's two heavy chains; IgM and IgE Fc regions contain three heavy chain constant domains (C_{H} domains 2-4) in each polypeptide chain. The presence of a Fc region in a chimeric immune complex should confer immunoglobulin effector functions to the complex, such as the ability to mediate the specific lysis of cells in the presence of complement. The heavy chain constant region domains of the immunoglobulins confer various properties known as antibody effector functions on a particular molecule containing that domain. Example effector functions include complement fixation, placental transfer, binding to staphylococcal protein, binding to streptococcal protein G, binding to mononuclear cells, neutrophils or mast cells and basophils. The association of particular domains and particular immunoglobulin isotypes with these effector functions is well known and for example, described in Immunology, Roitt et al., Mosby St. Louis, Mo. (1993 3rd Ed.) In addition, binding of the Fc of IgG1 to the FcRn should allow the immunoadhesins to persist in the circulation much longer (Ober, R. J., Martinez, C., Vaccaro, C., Zhou, J. & Ward, E. S. Visualizing the Site and Dynamics of IgG Salvage by the MHC Class I-Related Receptor, FcRn. J Immunol 172, 2021-2029 (2004)). This may allow the antitoxin to be used as a prophylactic. By contrast Iga₄ antibodies are usually associated with a decrease in allergy symptoms. This is likely to be due, at least in part, to the Fc of IgG₄ having an allergen-blocking effect at the mast cell level and/or at the level of the antigen-presenting cell (preventing IgE-facilitated activation of T cells). In addition, the favorable association reflects the enhanced production of IL-10 and other anti-inflammatory cytokines, which drive the production of IgG4. The existence of the IgG4 subclass, its up-regulation by anti-inflammatory factors and its own anti-inflammatory characteristics may help the immune system to dampen inappropriate inflammatory reactions, mediated through Fcλ, receptors such as those associated with symptoms of allergy and cytokine storm-associated pathology such as ARDS (Aalberse R C, et al., Clin Exp Allergy. 2009 April; 39(4):469-77. doi: 10.1111/j.1365-2222.2009.03207.x. Epub 2009 Feb. 13). A complex interplay of factors, including sequence differences in the IgG₄, Cλ2 domain, length and sequence variation in the hinge, the disposition of the Fabs relative to the Fc region, glycosylation in both IgG and receptor, and sequence variation between receptors, all play a role in the interaction between IgG and FcλRs Thus, for example, affinity constants (KA) for the interactions between IgG and the various Fcλ, receptors range from undetectable levels for IgG4 and FcλRIIIb, to 6.5 9 10⁷ M¹ for IgG1 and FcλRI (Bruhns P, et al. Specificity and affinity of human Fcλ, receptors and their polymorphic variants for human IgG subclasses. Blood 2009; 113:3716-3725.)

Portion of an Immunoglobulin light chain: As used herein, the term refers to that region of a light chain which is necessary for increasing stability of the described chimeric receptor protein and thus increasing production yield. Typically, this includes at least a portion of the immunoglobulin light chain constant region.

Heavy chain constant region: A polypeptide that contains at least a portion of the heavy chain immunoglobulin constant region. Typically, in its native form, IgG, IgD and IgA immunoglobulin heavy chain contain three constant regions joined to one variable region. IgM and IgE contain four constant regions joined to one variable region. As described herein, the constant regions are numbered sequentially from the region proximal to the variable domain. For example, in IgG, IgD, and IgA heavy chains, the regions are named as follows: variable region, constant region 1, constant region 2, constant region 3. For IgM and IgE, the regions are named as follows: variable region, constant region 1, constant region 2, constant region 3 and constant region 4.

Chimeric immunoglobulin heavy chain: An immunoglobulin derived heavy chain wherein at least a first portion of its amino acid sequence is a first antibody isotype or subtype and second peptide, polypeptide or protein or glycoprotein. The second polypeptide, protein or glycoprotein, may itself be derived from an immunoglobulin heavy chain of a different isotype or subtype antibody. Typically, a chimeric immunoglobulin heavy chain has its amino acid residue sequences derived from at least two different isotypes or subtypes of immunoglobulin heavy chain.

J chain: A polypeptide that is involved in the polymerization of immunoglobulins and transport of polymerized immunoglobulins through epithelial cells. See, The Immunoglobulin Helper: The J Chain in Immunoglobulin Genes, at pg. 345, Academic Press (1989). J chain is found in pentameric IgM and dimeric IgA and typically attached via disulfide bonds. J chain has been studied in both mouse and human.

Secretory component (SC): A component of secretory immunoglobulins that helps to protect the immunoglobulin against inactivating agents thereby increasing the biological effectiveness of secretory immunoglobulin. The secretory component may be from any mammal or rodent including mouse or human.

Linker: As used herein, the term refers to any polypeptide sequence used to facilitate the folding, stability or potency of a recombinantly produced polypeptide. Preferably, this linker is a flexible linker, for example, one composed of a polypeptide sequence such as (Gly₃Ser)₃ or (Gly₄Ser)₃. A linker may be interposed between two functional regions of an immunoadhesin for example between the Fc of an immunoglobulin and a functional ligand for example an ACE2 receptor sequence that binds to the Spike protein of SARS-CoV-2. A list of polypeptide linkers useful in the compositions and methods of the present disclosure is provided in Table 1 below. The corresponding nucleotide sequences for such linkers is easily determined from well characterized codon tables, which may also list codon preferences by species as well.

TABLE 1
Polypeptide linkers

| | SEQ ID |
|---|---|
| Linker Amino Acid Sequence | NO: |
| GGGGS | 23 |
| also referred to as (Gly₄Ser) | |
| GGGGSGGGGS | 24 |
| also referred to as (Gly₄Ser)₂ | |
| GGGGSGGGGSGGGGS | 25 |
| also referred to as (Gly₄Ser)₃ | |
| GGGS | 26 |
| also referred to as (Gly₃Ser) | |
| GGGSGGGS | 27 |
| also referred to as (Gly₃Ser)₂ | |
| GGGSGGGSGGGS | 28 |
| also referred to as (Gly₃Ser)₃ | |
| GGGGSGGGGSGGGGSGGGGSGGGGSASGGGGSGGGGSGGGGSGGGGSGGGGSGGGGS | 29 |
| AGGGSGGGGSGGGGSGGGGSGGGGSGGGGSGGGGS | 30 |
| AGGGSGGGGSGGGGSGGGGSGGGGSGGGGSGGGGSGGGGS | 31 |
| AGGGSGGGGSGGGGSGGGGSGGGGSGGGGSGGGGSGGGGSGGGGS | 32 |
| EGKSSGSGSESKEF | 33 |
| AGSGGSGGSGGSPVPSTPPTNSSSTPPTPSPSPVPSTPPTNSSSTPPTPSPSPVPSTPPTNS | 34 |
| SSTPPTPSPS | |
| AGSGGSGGSGGSPVPSTPPTPSPSTPPTPSPSPVPSTPPTNSSSTPPTPSPSPVPSTPPTPS | 35 |
| PSTPPTPSPS | |
| AGSGGSGGSGGSPVPSTPPTPSPSTPPTPSPSGGSGNSSGSGGSPVPSTPPTPSPSTPPTPS | 36 |
| PS | |
| AGSGGSGGSGGSPVPSTPPTPSPSTPPTPSPSPVPSTPPTPSPSTPPTPSPSPVPSTPPTPS | 37 |
| PSTPPTPSPS | |
| AGSGGSGGSGGSPVPSTPPTPSPSTPPTPSPSIQRTPKIQVYSRHPAENGKSNFLNCYVSGF | 38 |
| HPSDIEVDLLKNGERIEKVEHSDLSFSKDWSFYLLYYTEFTPTEKDEYACRVNHVTLSQPKI | |
| VKWDRDPVPSTPPTPSPSTPPTPSPS | |
| AGSGGSGGSGGSPVPSTPPTPSPSTPPTPSPSIQRTPKIQVYSRHPAENGKSNFLNCYVSGF | 39 |
| HPSDIEVDLLKNGERIEKVEHSDLSFSKDWSFYLLYYTEFTPTEKDEYACRVNHVTLSQPKI | |
| VKWDRDGGSGGSGGSGGS | |
| AGPVPSTPPTPSPSTPPTPSPSIQRTPKIQVYSRHPAENGKSNFLNCYVSGFHPSDIEVDLL | 40 |
| KNGERIEKVEHSDLSFSKDWSFYLLYYTEFTPTEKDEYACRVNHVTLSQPKIVKWDRDGGSG | |
| GSGGSGGSIQRTPKIQVYSRHPAENGKSNFLNCYVSGFHPSDIEVDLLKNGERIEKVEHSDL | |
| SFSKDWSFYLLYYTEFTPTEKDEYACRVNHVTLSQPKIVKWDRDGGSGGSGGSG | |
| AGSGGSGGSGGSPVPSTPPTPSPSTPPTPSPSIQRTPKIQVYSRHPAENGKSNFLNCYVSGF | 41 |
| HPSDIEVDLLKNGERIEKVEHSDLSFSKDWSFYLLYYTEFTPTEKDEYACRVNHVTLSQPKI | |
| VKWDRDGGSGGSGGSGGSIQRTPKIQVYSRHPAENGKSNFLNCYVSGFHPSDIEVDLLKNGE | |
| RIEKVEHSDLSFSKDWSFYLLYYTEFTPTEKDEYACRVNHVTLSQPKIVKWDRDPVPSTPPT | |
| PSPSTPPTPSPS | |
| AGSGGSGGSGGSPVPSTPPTPSPSTPPTPSPSQIFVKTLTGKTITLEVEPSDTIENVKAKIQ | 42 |
| DKEGIPPDQQRLIFAGKQLEDGRTLSDYNIQKESTLHLVLRLRGGGGSGGSGGSGGS | |
| AGSGGSGGSGGSPVPSTPPTPSPSTPPTPSPSDGRYSLTYIYTGLSKHVEDVPAFQALGSLN | 43 |
| DLQFFRYNSKDRKSQPMGLWRQVEGMEDWKQDSQLQKAREDIFMETLKDIVEYYNDSNGSHV | |
| LQGRFGCEIENNRSSGAFWKYYYDGKDYIEFNKEIPAWVPFDPAAQITKQKWEAEPVYVQRA | |
| KAYLEEECPATLRKYLKYSKNILDRQDPPSVVVTSHQAPGEKKKLKCLAYDFYPGKIDVHWT | |
| RAGEVQEPELRGDVLHNGNGTYQSWVVVAVPPQDTAPYSCHVQHSSLAQPLVVPWEASPVPS | |
| TPPTPSPSTPPTPS | |
| AGSGGSGGSGGSGGSGGSGGSGGSDGRYSLTYIYTGLSKHVEDVPAFQALGSLNDLQFFRYN | 44 |
| SKDRKSQPMGLWRQVEGMEDWKQDSQLQKAREDIFMETLKDIVEYYNDSNGSHVLQGRFGCE | |
| IENNRSSGAFWKYYYDGKDYIEFNKEIPAWVPFDPAAQITKQKWEAEPVYVQRAKAYLEEEC | |
| PATLRKYLKYSKNILDRQDPPSVVVTSHQAPGEKKKLKCLAYDFYPGKIDVHWTRAGEVQEP | |
| ELRGDVLHNGNGTYQSWVVVAVPPQDTAPYSCHVQHSSLAQPLVVPWEASGGSGGSGGSGGS | |
| DGRYSLTYIYTGLSKHVEDVPAFQALGSLNDLQFFRYNSKDRKSQPMGLWRQVEGMEDWKQD | |
| SQLQKAREDIFMETLKDIVEYYNDSNGSHVLQGRFGCEIENNRSSGAFWKYYYDGKDYIEFN | |
| KEIPAWVPFDPAAQITKQKWEAEPVYVQRAKAYLEEECPATLRKYLKYSKNILDRQDPPSVV | |
| VTSHQAPGEKKKLKCLAYDFYPGKIDVHWTRAGEVQEPELRGDVLHNGNGTYQSWVVVAVPP | |
| QDTAPYSCHVQHSSLAQPLVVPWEASPVPSTPPTPSPSTPPTPSPS | |
| AGSGNSSGSGGSGGSGNSSGSGGSPVPSTPPTPSPSTPPTPSPS | 45 |
| KLSGGGGSGGGGSGGGGSAEAWYNLGNAYYKQGDYQKAIEYYQKALELDPNNAEAWYNLGNA | 46 |
| YYKQGDYQKAIEYYQKALELDPNNAEAWYNLGNAYYKQGDYQKAIEDYQKALELDPNNLQRS | |
| AGGGGSGGGGSGGGG | |
| KLSGGGGSGGGGSGGGGSAEAWYNLGNAYYKQGDYQKAIEYYQKALELDPNNAEAWYNLGNA | 47 |
| YYKQGDYQKAIEYYQKALELDPNNAEAWYNLGNAYYKQGDYQKAIEDYQKALELDPNNLQAE | |
| AWKNLGNAYYKQGDYQKAIEYYQKALELDPNNASAWYNLGNAYYKQGDYQKAIEYYQKALEL | |
| DPNNAKAWYRRGNAYYKQGDYQKAIEDYQKALELDPNNRSRSAGGGGSGGGGSGGGG | |
| KLSGGGGSGGGGSGGGGSAEAWYNLGNAYYKQGDYQKAIEYYQKALELDPNNAEAWYNLGNA | 48 |
| YYKQGDYQKAIEYYQKALELDPNNAEAWYNLGNAYYKQGDYQKAIEDYQKALELDPNNLQAE | |
| AWKNLGNAYYKQGDYQKAIEYYQKALELDPNNASAWYNLGNAYYKQGDYQKAIEYYQKALEL | |
| DPNNAKAWYRRGNAYYKQGDYQKAIEDYQKALELDPNNRSAEAWYNLGNAYYKQGDYQKAIE | |
| YYQKALELDPNNAEAWYNLGNAYYKQGDYQKAIEYYQKALELDPNNAEAWYNLGNAYYKQGD | |
| YQKAIEDYQKALELDPNNLQRSAGGGGSGGGGSGGGG | |
| KLSGGGGSGGGGSGGGGSAEAWYNLGNAYYKQGDYQKAIEYYQKALELDPNNAEAWYNLGNA | 49 |
| YYKQGDYQKAIEYYQKALELDPNNAEAWYNLGNAYYKQGDYQKAIEDYQKALELDPNNLQAE | |
| AWKNLGNAYYKQGDYQKAIEYYQKALELDPNNASAWYNLGNAYYKQGDYQKAIEYYQKALEL | |
| DPNNAKAWYRRGNAYYKQGDYQKAIEDYQKALELDPNNRSAEAWYNLGNAYYKQGDYQKAIE | |
| YYQKALELDPNNAEAWYNLGNAYYKQGDYQKAIEYYQKALELDPNNAEAWYNLGNAYYKQGD | |
| YQKAIEDYQKALELDPNNLQAEAWKNLGNAYYKQGDYQKAIEYYQKALELDPNNASAWYNLG | |
| NAYYKQGDYQKAIEYYQKALELDPNNAKAWYRRGNAYYKQGDYQKAIEDYQKALELDPNNRS | |
| AGGGGSGGGGSGGGG | |

Transgenic plant: Genetically engineered plant or progeny of genetically engineered plants. The transgenic plant usually contains material from at least one unrelated organism, such as a virus, bacterium, fungus, another plant or animal.

Plant Material: Materials derived from plants including, plant cell walls, plant organelles, plant cytoplasm, intact plant cells, plant tissues, plant leaves, plant stems, plant roots, plant seeds, and viable plants.

Monocots: Flowering plants whose embryos have one cotyledon or seed leaf. Examples of monocots are: lilies; grasses; corn; grains, including oats, wheat and barley; orchids; irises; onions and palms.

Dicots: Flowering plants whose embryos have two seed halves or cotyledons. Examples of dicots are: tobacco; tomato; the legumes including alfalfa; oaks; maples; roses; mints; squashes; daisies; walnuts; cacti; violets and buttercups.

Glycosylation: The modification of a protein by oligosaccharides. See, Marshall, Ann. Rev. Biochem., 41:673 (1972) and Marshall, Biochem. Soc. Symp., 40:17 (1974) for a general review of the polypeptide sequences that function as glycosylation signals. These signals are recognized in both mammalian and in plant cells.

Plant-specific glycosylation: The glycosylation pattern found on plant-expressed proteins, which is different from that found in proteins made in mammalian or insect cells. Proteins expressed in plants or plant cells have a different pattern of glycosylation than do proteins expressed in other types of cells, including mammalian cells and insect cells. Detailed studies characterizing plant-specific glycosylation and comparing it with glycosylation in other cell types have been performed by Cabanes-Macheteau et al., Glycobiology 9(4):365-372 (1999), Lerouge et al., Plant Molecular Biology 38:31-48 (1998) and Altmann, Glycoconjugate J. 14:643-646 (1997). Plant-specific glycosylation generates glycans that have xylose linked β(1,2) to mannose. Neither mammalian nor insect glycosylation generate xylose linked β(1,2) to mannose. Plants do not have a sialic acid linked to the terminus of the glycan, whereas mammalian cells do. In addition, plant-specific glycosylation results in a fucose linked α(1,3) to the proximal GlcNAc, while glycosylation in mammalian cells results in typically a fucose linked α(1,6) to the proximal GlcNAc.

Wild Type: As used herein the term “wild type” referring to glycosylation of proteins produced in plants including but not limited N. benthamiana, means plant-specific glycosylation wherein glycans have xylose linked β(1,2) to mannose, and fucose linked α(1,3) to the proximal GlcNAc.

DXT/FT: The term “DXT/FT” means a plant, including but not limited to N. benthamiana in which the endogenous β1,2-xylosyltransferase (XylT) and α1,3-fucosyltransferase (FucT) genes have been substantially down-regulated or eliminated by any method including but not limited to RNA interference. Glycoproteins produced in DXT/FT plants, including those produced in N. benthamiana, contain almost homogeneous N-glycan species without detectable plant-specific β1,2-xylose and α1,3-fucose residues.

Humanized: The term “humanized” referring to glycosylation of proteins produced in plants refers to glycoproteins produced in DXT/FT plants that have been modified to add terminal β1,4-Gal residues to N-glycan. This may be accomplished by prior infiltration into the plant or by co-infiltration with a binary vector that encodes a modified human β1,4-galactosyl-transferase (ST-Gaff).

Immunoglobulin Heavy Chain: The chimeric ACE2 and modified or altered ACE2 receptor proteins contain at least a portion of an immunoglobulin heavy chain constant region sufficient to confer either the ability to multimerize the attached anthrax receptor protein, confer antibody effector functions, stabilize the chimeric protein in the plant, confer the ability to be purified by Protein A or G, or to improve pharmacokinetics. These properties are conferred by the constant regions of the immunoglobulin heavy chains. If the chimeric toxin receptor protein contains only an immunoglobulin heavy chain, the portion of the heavy chain in the immunoglobulin complex preferably contains at least domains CH2 and CH3 and more preferably, only CH2 and CH3. If the chimeric toxin receptor protein contains both a heavy chain and a light chain, the portion of the heavy chain in the immunoglobulin complex preferably also contains a CH1 domain. One of skill in the art will readily be able to identify immunoglobulin heavy chain constant region sequences. For example, a number of immunoglobulin DNA and protein sequences are available through public sequence databases such as GenBank and UniProt. Table 2 shows the Accession numbers that can be used to obtain amino acid sequences and the encoding nucleic acid sequences for various human immunoglobulin heavy chains useful in preparing ACE2-Fc fusions of the present disclosure. Additionally, it is contemplated that the ACE2-Fc fusion proteins of the present disclosure can comprise a fragment or portion of a human immunoglobulin heavy chain that comprises the Fc region. The amino acid sequences of exemplary heavy chain Fc region fragments useful in the fusions of the present disclosure are also provided in Table 2 (SEQ ID NOs: 18-22) and the accompanying Sequence Listing.

TABLE 2

| ACCESSION NO. | Human IgG Type Heavy Chain |
|---|---|
| J00220 | IgA1 Heavy Chain Constant Region |
| J00221 | IgA2 Heavy Chain Constant Region |
| J00228 | IgG1 Heavy Chain Constant Region |
| J00230 | IgG2 Heavy Chain Constant Region |
| X03604 | IgG3 Heavy Chain Constant Region |
| K01316 | IgG4 Heavy Chain Constant Region |
| K02876 | IgD Heavy Chain Constant Region |
| K02877 | IgD Heavy Chain Constant Region |
| K02878 | Germline IgD Heavy Chain |
| K02879 | Germline IgD Heavy Chain C-S-3 Domain |
| K01311 | Germline IgD Heavy Chain J-S Region: C-S CH1 |
| K02880 | Germline IgD Heavy Chain Gene, C-Region, Secreted Terminus |
| K02881 | Germline IgD-Heavy Chain Gene, C-Region, First Domain of |
| | Membrane Terminus |
| K02882 | Germline IgD Heavy Chain |
| K02875 | Germline IgD Heavy Chain Gene, C-Region, C-S-1 Domain |
| L00022 | IgE Heavy Chain Constant Region |
| X17115 | IgM Heavy Chain Complete |
| Heavy Chain | |
| Fc Fragments | Fc Fragment Sequence |
| Fc (IgG1) or “Fc1” | EPKS ADKTHTCPPC PAPELLGGPS VFLFPPKPKD TLMISRTPEV |
| (SEQ ID NO: 18) | TCVVVDVSHE DPEVKFNWYV DGVEVHNAKT KPREEQYNST YRVVSVLTVL |
| | HQDWLNGKEY KCKVSNKALP APIEKTISKA KGQPREPQVY TLPPSRDELT |
| | KNQVSLTCLV KGFYPSDIAV EWESNGQPEN NYKTTPPVLD SDGSFFLYSK |
| | LTVDKSRWQQ GNVFSCSVMH EALHNHYTQK SLSLSPGK |
| Fc (IgG4) or “Fc4” | ESKY GPPCPPCPAP EAAGGPSVFL FPPKPKDTLM ISRTPEVTCV |
| (SEQ ID NO: 19) | VVDVSQEDPE VQFNWYVDGV EVHNAKTKPR EEQFNSTYRV VSVLTVLHQD |
| | WLNGKEYKCK VSNKGLPSSI EKTISKAKGQ PREPQVYTLP PSQEEMTKNQ |
| | VSLTCLVKGF YPSDIAVEWE SNGQPENNYK TTPPVLDSDG SFFLYSRLTV |
| | DKSRWQEGNV FSCSVMHEAL HNHYTQKSLS LSLGK |
| Fc (IgG3) or “Fc3” | EPKS CDKTHTCPPC PAPELLGGPS VFLFPPKPKD TLMISRTPEV |
| (SEQ ID NO: 20) | TCVVVDVSHE DPEVQFKWYV DGVEVHNAKT KPREEQYNST FRVVSVLTVL |
| | HQDWLNGKEY KCKVSNKALP APIEKTISKT KGQPREPQVY TLPPSREEMT |
| | KNQVSLTCLV KGFYPSDIAV EWESSGQPEN NYNTTPPMLD SDGSFFLYSK |
| | LTVDKSRWQQ GNIFSCSVMH EALHNHFTQK SLSLSPGK |
| Fc (IgA1) | PVPS TPPTPSPSTP PTPSPSCCHP RLSLHRPALE DLLLGSEAQL |
| (SEQ ID NO: 21) | TCTLTGLRDA SGVTFTWTPS SGKSAVQGPP ERDLCGCYSV SSVLPGCAEP |
| | WNHGKTFTCT AAYPESKTPL TATLSKSGNT FRPEVHLLPP PSEELALNEL |
| | VTLTCLARGF SPKDVLVRWL QGSQELPREK YLTWASRQEP SQGTTTFAVT |
| | SILRVAAEDW KKGDTFSCMV GHEALPLAFT QKTIDRLAGK |
| Fc (IgA2) | PVPP PPPCCHPRLS LHRPALEDLL LGSEAQLTCT LTGLRDASGA |
| (SEQ ID NO: 22) | TFTWTPSSGK SAVQGPPERD LCGCYSVSSV LPGCAQPWNH GETFTCTAAH |
| | PELKTPLTAQ ITKSGNTFRP EVHLLPPPSE ELALNELVTL TCLARGFSPK |
| | DVLVRWLQGS QELPRKYLTW ASRQEPSQGT TTFAVTSILR VAAEDWKKGD |
| | TFSCMVGHEA LPLAFTQKTI DRLAGK |

Moiety that that extends its half-life (T_{1/2}) or/and the duration of action of the receptor—is a moiety that when attached to Functional ACE2 can extend the circulation T_{1/2}, blood T_{1/2}, plasma T_{1/2}, serum T_{1/2}, terminal T_{1/2}, biological T_{1/2}, elimination T_{1/2} or functional T_{1/2}, or any combination thereof, of the Functional ACE2. Such attachment may be covalent or non-covalent. Examples of the Moiety include but are not limited to the Fc region of IgG₁, IgG2, IgG3, IgG₄, IgA₁, IgA₂, IgD, IgM or IgE antibody isotypes. The Fc region is composed constant domains 2 and 3 of the heavy chain of the forgoing antibody isotypes as is known in the art. Preferred are Fcs that have the ability to associate with one another either covalently or non-covalently to form a dimer.

Additional examples of the Moiety that extends its half-life (T_{1/2}) or/and the duration of action of the receptor include human serum albumin (HSA) or a portion thereof (e.g., domain III) that binds to the neonatal Fc receptor (FcRn). The HSA or FcRn-binding portion of HSA can optionally have one or more mutations that confer a beneficial property or effect. In some embodiments, the HSA or FcRn-binding portion thereof has one or more mutations that enhance pH-dependent HSA binding to FcRn or/and increase HSA half-life, such as K573P or/and E505G/V547A. A protracting moiety can be an unstructured polypeptide.

Yet further examples of a Moiety that extends the half-life (T1/2) or/and the duration of action of the receptor include a carboxy-terminal peptide (CTP) derived from the β-subunit of human chorionic gonadotropin (hCG). In the human body, the fourth, fifth, seventh and eight serine residues of the 34-aa CTP of hCG-p typically are attached to O-glycans terminating with a sialic acid residue. Such further examples also include A protracting moiety can be 1, 2, 3, 4, 5 or more units of a synthetic polymer. The synthetic polymer can be biodegradable or non-biodegradable. Biodegradable polymers useful as Moieties that extend the half-life (T_{1/2}) or/and the duration of action of the receptor include include, but are not limited to, poly(2-methacryloyloxyethyl phosphorylcholine) (PMPC) and poly[oligo(ethylene glycol) methyl ether methacrylate] (POEGMA). Non-biodegradable polymers useful as Moieties that extend the half-life (T_{1/2}) or/and the duration of action of the receptor include without limitation poly(ethylene glycol) (PEG), polyglycerol, poly(N-(2-hydroxypropyl)methacrylamide) (PHPMA), polyoxazolines and poly(N-vinylpyrrolidone) (PVP). A synthetic polymer can be polyethylene glycol (PEG). PEGylation can be done by chemical or enzymatic, site-specific coupling or by random coupling.

Modified ACE2 sequence specific to the ACE2 α-helix 1 region means particularly amino acid residues in positions 30 to 42 that have been modified such that the affinity of the resulting modified ACE2 for SARS-COV-2 is increased compared to the affinity of the sequence of SEQ ID NO: 1.

Preferred modified ACE2 sequence residues specific to the ACE2 α-helix 1 region means particular residues within this region that are preferred for modification to increase binding affinity including residues 30, 31, 34, 38, 40 and 42. Selected preferred modified ACE2 sequence residues specific to the ACE2 α-helix 1 are modifications of residue 30 which change it from D to E or S, residue 31 which change it from K to 0 or E, residue 34 which change it from H to S or V, residue 38 which change it from D to E, residue 40 which change it from F to S, or residue 42 which change it from Q to A and double modifications of the forgoing including modification of residue 38 from D to E and 40 from F to S,

Modified ACE2 sequence specific to the ACE2 loop 2, region means particularly amino acid residues in that have been modified such that the affinity of the resulting modified ACE2 for SARS-CoV-2 is increased compared to the affinity of the sequence of SEQ ID NO: 1.

Preferred modified ACE2 sequence residues specific to ACE2 loop 2, region means particular residues within this region that are preferred for modification to increase binding affinity including residues the positions 81 to 84 that may be modified for this effect. Selected preferred modified ACE2 sequence residues specific to the ACE2 loop 2 are modifications of residue 81 from Q to K, and 82 from M to N or K or 1″, and the double modification of residue 81 from Q to K and 82 from M to N.

Modified ACE2 sequence specific to the ACE2 β-sheet 5 region means particularly amino acids that have been modified such that the affinity of the resulting modified ACE2 for SARS-CoV-2 is increased compared to the affinity of the sequence of SEQ ID NO: 1.

Preferred modified ACE2 sequence residues specific to ACE2 β-sheet 5 region means particular amino acid residues within this region that are preferred for modification to increase binding affinity including residues in positions 353 to 357 that may be modified to this effect.

Selected preferred modified ACE2 sequence residues specific to the ACE2 p-sheet 5 region are modifications of residue 354 from G to H or K.

Modified ACE2 sequence specific to the ACE2 α-helix 10 region means particularly amino acids that have been modified such that the affinity of the resulting modified ACE2 for SARS-CoV-2 is increased compared to the affinity of the sequence of SEQ ID NO: 1.

Preferred modified ACE2 sequence residues specific to ACE2 α-helix 10 region means particular amino acid residues within this region that are preferred for modification to increase binding affinity including residues in positions 327 to 329 that may be modified to this effect.

Selected preferred modified ACE2 sequence residues specific to the ACE2 α-helix 10 region are modifications of residue 329 from E to N or K.

Effective amount: An effective amount of an immunoadhesin of the present invention is sufficient to detectably inhibit viral attachment, viral cellular cytopathology or cellular cytotoxicity, or infection of an animal or to reduce the severity or duration of infection or symptoms of infection.

Construct or Vector: An artificially assembled DNA segment to be transferred into a target tissue or cell of a plant or animal, especially a mammal. Typically, the construct will include the gene or genes of a particular interest, a marker gene and appropriate control sequences.

Plasmid “An autonomous, self-replicating extrachromosomal DNA molecule. Plasmid constructs containing suitable regulatory elements are also referred to as “expression cassettes.” In a preferred embodiment, a plasmid construct also contains a screening or selectable marker, for example an antibiotic resistance gene.

Selectable marker: A gene that encodes a product that allows the growth of transgenic tissue or cells on a selective medium. Non-limiting examples of selectable markers include genes encoding for antibiotic resistance, e.g., ampicillin, kanamycin, or the like. Other selectable markers will be known to those of skill in the art.

COVID-19, caused by the coronavirus SARS-CoV-2, is a newly emerging human health threat with a more than 2% case fatality rate. SARS-CoV-2 uses the cell surface protein ACE2 to enter and infect cells. Soluble recombinant human ACE2 binds to SARS-CoV-2 and inhibits infection of VERO cells, but the concentration required to achieve 50% inhibition is fairly high.

Three major strategies for improving the inhibitory potency of soluble human ACE2 against infection are (1) improve the binding of the receptor region of ACE2 with the SARS-CoV-2 spike protein, (2) link the ACE2 receptor to a moiety capable of extending its half-life (T_{1/2}) or/and the duration of action of the receptor and/or (3) when linked to such a moiety provide an orientation whereby the ACE2 receptor is freely accessed by the SARS-CoV-2 S glycoprotein.

Using a fusion of a modified ACE2 binding sequence (extracellular domain) and a moiety such as the Fc region of a human immunoglobulin the present invention provides a superior inhibitor of SARS-CoV-2 infection and a potency greater than the expected increased potency of ACE2-Fc due to the stoichiometry of ACE2 in the Fc fusion (two ACE2 binding domains per molecule). In addition to the improved potency, the modified ACE2-Fc is also expected to have superior pharmacokinetics, as Fc will confer a long circulating half-life and the ability to be delivered to airway mucosal surfaces, the site of SARS-CoV-2 infection. Unlike antibodies against SARS-CoV-2, a ACE2-Fc and the modified ACE2-Fc decoy of the invention will not subject the virus to selection for neutralization by SARS-CoV-2 escape mutants, as any mutation that decreases binding to the decoy will decrease binding to the native receptor on cells, resulting in an attenuated virus.

The anti-SARS-COV-2 inhibitory potency of the modified ACE2 fused to the Fc of different immunoglobulin isotypes including but not limited to—IgG1, IgG3, IgG4 IgA1 and IgA2—is increased compared to the same Fc fusions of unmodified ACE2. Fusions of Fc and the full-length ACE2 extracellular domain (amino acids 19-615) are described, and genetic constructs capable of expression by eukaryotic host cells, tissues organs or organisms are provided. Purified modified ACE2-Fc fusions and formulations thereof are also shown. The ability of the ACE2-Fc variants to bind the S1 domain of the SARS-CoV-2 spike protein in a functional ELISA as well as in cell culture is disclosed. In further preferred embodiments of the modified ACE2-Fc fusion, amino acid changes at specific positions in the human ACE2 and/or Fc are disclosed that further increase binding to the SARS-CoV-2 spike protein and increase SARS-Cov-2 viral inactivation in vitro and in vivo.

In a preferred, but not limiting embodiment, the modified ACE2-Fc fusion is expressed using a rapid transient plant expression system. Nucleotide sequences encoding the ACE2-Fc fusions are cloned into a plant expression vector and the constructs transformed into Agrobacterium tumefaciens (A. t). The Agrobacterium strains transiently transform Nicotiana benthamiana plants, which express the recombinant proteins. In a preferred embodiment vacuum infiltration is used to transport the A.t. into the tissues of plants.

Although the N-glycans in ACE2 do not make contact with the S1 RBD, proper N-glycosylation of the Fc may be important for in vivo viral neutralization. Accordingly, it is preferred to produce fusion proteins with N-glycans as similar to typical mammalian N-glycans as possible using an N. benthamiana line in which the endogenous R1,2-xylosyltransferase (XylT) and α1,3-fucosyltransferase (FucT) genes have been down-regulated by RNA interference. Such strains are produced as described in (Strasser et al. 2008). Glycoproteins produced in this line contain almost homogeneous N-glycan species without detectable plant-specific β1,2-xylose and α1,3-fucose residues. To ensure uniform addition of terminal β1,4-Gal residues to N-glycans, it is additionally preferred to co-infiltrate this N. benthamiana with a binary vector that encodes a modified human β1,4-galactosyl-transferase (ST-GalT) to “humanize” plant-made N-glycans (Strasser et al. 2009).

After a suitable period of time the plant-produced fusion proteins are purified from extracts of plant tissue using standard procedures, including Protein A affinity chromatography in the case of ACE2-IgG Fc fusions. The plant-produced recombinant modified ACE2-Fc fusion proteins are assayed for binding to the recombinant S glycoprotein of SARS-CoV-2 and evaluated in vitro and in vivo for SARS-CoV-2 neutralizing activity.

Genetic fusions of human ACE2 with human immunoglobulin sequences, and preferably immunoglobulin Fc sequences, which include the hinge, CH2 and CH3 of IgG1, IgG3, IgG4, IgA1 and IgA2 are produced. The ACE2 of the fusion is functional in its ability to bind to the SARS-CoV-2 S glycoprotein, and such functional ACE2 include, for example those incorporating the full-length ACE2 extracellular domain (amino acids 19-740 of SEQ ID NO: 1) or shorter sequence of the extracellular domain (amino acids 19-615 of SEQ ID NO: 1), additional variants including modified ACE2 such as fragments of the soluble ACE2 sub-domain that comprise the minimum sequence of amino acids required to bind the SARS-CoV-2 spike protein. Such functional ACE2 proteins may be fused with human immunoglobulin sequences. The full length functional ACE2 extracellular domain (amino acids 19-740) has been expressed in mammalian cells in a form that retains both its enzymatic activity and its ability to bind the SARS-CoV-2 S glycoprotein. Such full length functional ACE2 extracellular domains and Fc fusions thereof can be difficult to express in recombinant hosts resulting in low yield due to instability during processing. Amino acid sequences within the 19-740 full length functional ACE2 extracellular domain are sites where the full length sequence is labile and breaks into fragments that disrupt the functionality of the molecule or the integrity of the fusion molecule and may lead to the accumulation of peptide fragments that must be removed during further processing and purification thereby decreasing yield and increasing costs of production. By using a shorter sequence of the ACE2 receptor extracellular domain (for example amino acids 19-614 or 19-615 of SEQ ID NO: 1) these labile sequences are eliminated in the both the functional ACE2 and in Fc fusions thereof resulting in the expression in the recombinant host of a more stable protein product that can be processed and purified with higher yield and greater purity.

The foregoing soluble ACE2 full length amino acid sequences and shorter sequence of the extracellular domain may be further modified at specific amino acid residues which are involved in binding to the SARS-CoV-2 spike protein. Such amino acid residues which are involved in binding to the SARS-CoV-2 spike protein may be referred to as receptor binding domain contact sequences.

Using the amino acid numbering of SEQ ID NO: 1 (shown in FIG. 1), four general regions on ACE2 have been identified to be important for binding SARS-CoV S glycoprotein: (i) amino acid residues within the ACE2 α-helix 1; (ii) amino acid residues within the ACE2 loop 2; (iii) amino acid residues within ACE2 β-sheet 5 (Han, Penn-Nicholson, and Cho 2006) and amino acid residues within ACE2 α-helix 10. Among the amino acids with the forgoing regions of ACE2 particular residues appear to play a role in binding to the SARS-CoV-2 spike protein: (i) residues K31 and Y41 on the ACE2 α-helix 1; (ii) Q81, M82, Y83 and P84 on the ACE2 loop 2; and (iii) K353, D355 and R357 on ACE2 β-sheet 5 (Han, Penn-Nicholson, and Cho 2006) and residue E329 on ACE2 α-helix 10. This analysis was based on crystal X-ray crystallography of SARS-CoV S glycoprotein bound to human ACE2. Among these amino acids, K31 and K353 in hACE2 appear to be the most critical residues for recognition of the SARS-2 spike protein Receptor Binding Motif (a smaller sub-region of the same protein's RBD), while additional amino acids, D30, H34, D38 and Q42 on the ACE2 α-helix 1 and E329 on the ACE2 α-helix 10, have also been identified as important for SARS-CoV-2 RBD binding by structural modeling (Liu et al. 2020). Modification of specific amino acid residues within these four regions are expected to achieve increased binding affinity of the ACE2 receptor for the SARS-CoV spike protein. Recent analysis of the SARS-CoV-2 S glycoprotein has determined that this new coronavirus, like SARS-CoV-1, also uses ACE2 as its receptor (Wan et al. 2020). A simulation of the structure of SARS-CoV-2 spike protein structure (Liu et al. 2020) was used to determine the probable contact amino acids on ACE2 for that S protein. This suggests that the interaction of SARS-CoV-2 with ACE2 is generally similar to the interaction of SARS-COV with ACE2. Residues 31, 41, 82, 353, 355, and 357 on ACE2 locate in the interface when interacting with SARS-CoV-2 spike protein (Liu et al. 2020).

Modification of the amino acid sequence of the ACE2 α-helix 1 region particularly amino acid residues in positions 30 to 42 may increase binding to the SARS-COV-2 spike protein. Particular residues within this region that are preferred for modification to increase binding affinity are residues 30, 3′1 34, 38, 40 and 42. Preferred are modifications of residue 30 from D to E or S, residue 31 K to Q or E, residue. 34 H to S or V, residue. 38 D to E, residue 40 F to S, or residue 42 Q to A. Single modifications of residues within this region are provided, as well as double modifications in which 2 or more of the specific foregoing residues in positions 30 to 42 are made, for example the double modification of residue 38 D to E and residue 40 F to S.

Modification of the amino acid sequence of the ACE2 loop 2, region particularly amino acid residues in positions 81 to 84 may be modified for this effect. Examples of preferred single modifications are of residue 82 M to N or K or T and preferred double modifications including modifications to residue 82 M tip N and residue 81 Q to K. A recent molecular modeling study found that the N82 of pangolin ACE2 showed closer contact (1.621 Å) with F486 of SARS-CoV-2 S protein than the M82 of human ACE2 (3.753 Å) (31).

Modification of the amino acid sequence of the ACE2 β-sheet 5 region particularly residue 329 and residues in positions spanning 353 to 357, may be modified for the effect of increasing affinity of ACE2 for SARS-COV-2 spike protein wherein 354 is preferred. Preferred are modifications of residue 329 E to N or K and residue 354 G to H or K.

Modification of the amino acid sequence of the ACE2 α-helix 10 region, particularly residue 329, may be made for the effect of increasing affinity of ACE2 for SARS-COV-2 spike protein, Preferred are modifications of residue 329 E to N or K

Several amino acid variants of human ACE2 that have an amino acid other that the one occurring in the wild-type ACE2 amino acid sequence have been identified in the literature and may have greater affinity for SARS-CoV2 spike protein. When such human variant ACE2 is joined with Fc (with or without a linker sequence), the increased affinity of the variant ACE2 sequence for SARS-CoV-2 spike protein would provide the human variant ACE2-Fc with measurable increase in binding to SARS-CoV-2 virus and improved virus neutralization in vitro and in vivo. Such improved virus neutralization would be expected to reduce the severity of symptoms of COVID-19 infection in individuals treated with the human variant ACE2-Fc. In addition, such human variant ACE2 sequences may be combined with additional altered sequences such as H34S to produce ACE2 sequences with even greater affinity for SARS-CoV-2 spike protein. When such altered ACE2 sequence is combined with human variant ACE2 and joined with Fc (with or without a linker sequence), additional increased affinity of the human variant ACE2 sequence and combined altered sequence for SARS-CoV-2 spike protein would provide further measurable increase in binding to SARS-CoV-2 virus and further improved virus neutralization in vitro and in vivo.

In addition, the presence or absence of sites for N-glycosylation of ACE2 may alter the affinity of ACE2 for binding of SARS-CoV 2 spike protein. For example, by changing the amino acid sequence of residue 103 from N to S the glycosylation site thereon can be removed and the resulting ACE2 glycosylation variant's affinity for SARS-CoV 2 spike protein altered. Similarly, alterations of the residues 547 and 548 adjacent to and nearby residue 546 will remove glycosylation of residue 546. Furthermore, alteration of residue 546 changing N to S removes glycosylation from residue 546.

The activities of these ACE2-receptor variants may be characterized in vitro by binding assays, such as ELISAs, or cell-based assays such as inhibition of cytopathological effect caused by SARS-CoV-2 infection of cells in the presence of soluble ACE2, ACE2-Fc, modified soluble ACE2 and modified ACE2-immunoadhesins such as modified ACE2-Fc.

The structural integrity of the ACE2-Fc proteins according to the invention is determined by reducing and non-reducing SDS-PAGE and immunoblotting with Fc-specific and ACE2-specific antibodies. Protein size is determined by analytical size exclusion chromatography. The ability of the ACE2-Fc variants to bind the S1 domain of the SARS-CoV-2 spike protein is determined in a functional ELISA. The effect of making single or multiple amino acid changes at specific positions in the human ACE2 sequence of our fusion proteins, and their binding to the spike protein is also determined by these techniques.

All ACE2-Fc variants that specifically bind to S protein of SARS-CoV-2 are tested for the ability to block infection of mammalian cells by SARS-CoV-2. The recombinant ACE2 and modified ACE2 immunoglobulin Fc fusion proteins with inhibitory activity against the SARS-CoV-2 binding are further tested for their antiviral activity against live SARS-CoV-2 infection both in vitro and in vivo in mice transgenic for the human angiotensin-converting enzyme 2 virus receptor, an animal model of the disease ((Tseng et al. 2007), which is herein incorporated by reference.)

In U.S. Pat. No. 7,951,378, herein incorporated by reference, it has been demonstrated, with human rhinovirus (HRV), and its cellular receptor, intercellular adhesion molecule 1 (ICAM1), that an ICAM-Fc fusion is a significantly more potent inhibitor of HRV infection than soluble ICAM. Like ACE2, ICAM1 is found on cells lining the portions of the respiratory tract. Recombinant soluble ICAM1 (sICAM1) inhibits HRV infection of susceptible cells, with an in vitro EC₅₀ (50% inhibition of the virus' cytopathic effect) of ˜3 μg/ml against a standard HRV serotype. However, fusions of ICAM1 to human Fc are more potent and have significantly lower EC₅₀. Recombinant ICAM1-IgA2Fc, produced in our plant expression system, had an EC₅₀ of 0.5 μg/ml, while ICAM1-IgG1 Fc had an EC₅₀ of 0.3 μg/ml. An ICAM1-IgA1 Fc had an EC₅₀ of 0.08 μg/ml (Martin et al. 1993).

These differences in in vitro virus neutralization may be related to structural differences in the immunoglobulin isotypes. For instance, studies of IgA1 and IgA2 in solution indicate that they have more of a T-shape than the Y shape typical of IgG. The arms of the T in IgA1 are more extended, due to its longer hinge, than the arms of IgA2 (Boehm et al. 1999; Furtado et al. 2004). Structural modeling indicates that the Fab-to-Fab center-to-center distance is 8.2 nm in IgA2,16.9 nm in IgA1 and 7 to 9 nm in IgG, depending on the subtype (Boehm et al. 1999; Eryilmaz et al. 2013). Thus, in a preferred embodiment significant increases in potency can be engineered into a ACE2-Fc fusion against SARS-CoV-2 by using different Fc fusions from other immunoglobulin isotypes, such as IgG1, IgG2, IgG3, IgG4, IgA1, IgA2, IgE, and IgM. Furthermore, ACE2-Fc may not just block SARS-CoV-2 virus binding to the cell, but multiple ACE2 ligands bound to the virus may trigger disruption of the viral particle and non-productive release of viral nucleic acid, as has been seen with ICAM-Fc disruption of HRV (Martin et al. 1993; Casasnovas and Springer 1994).

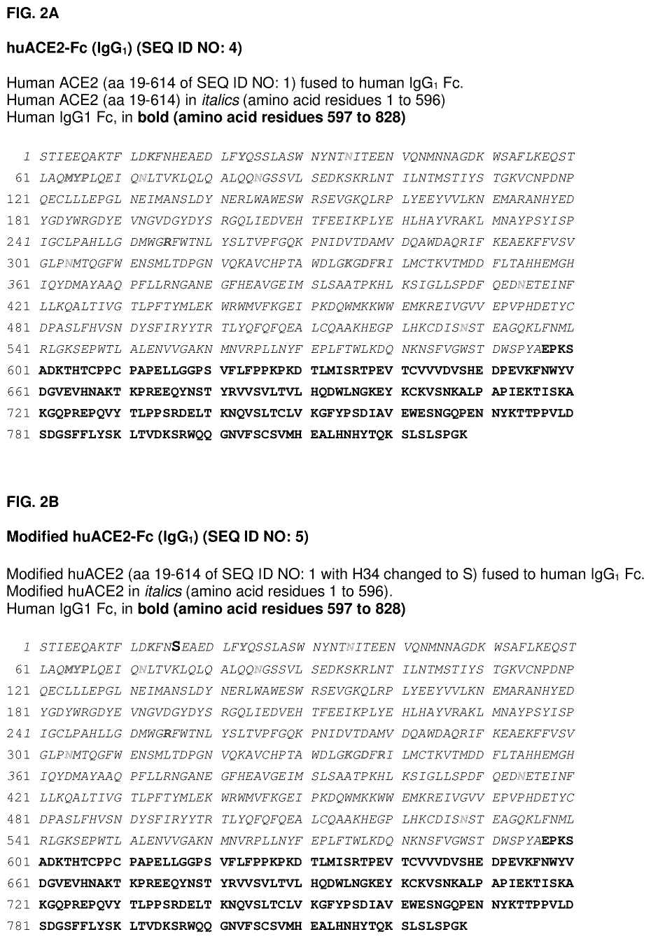

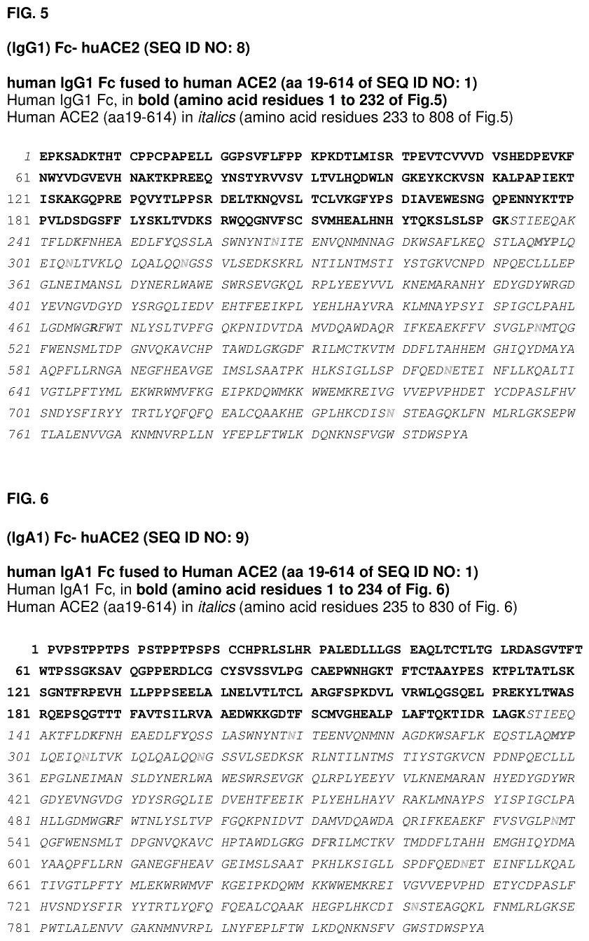

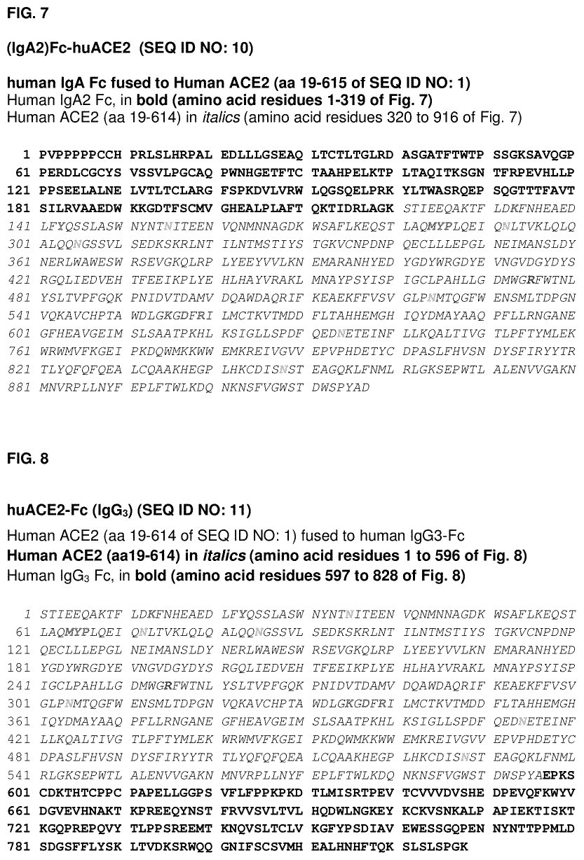

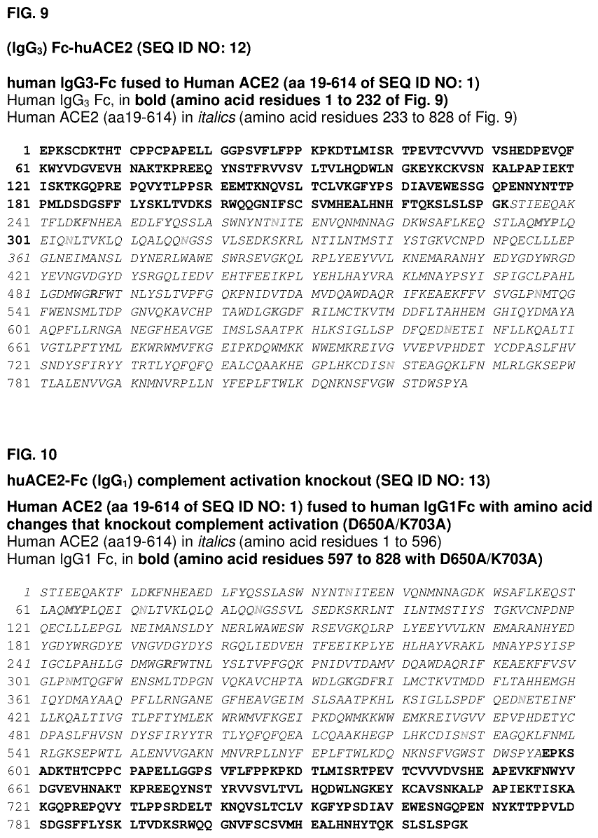

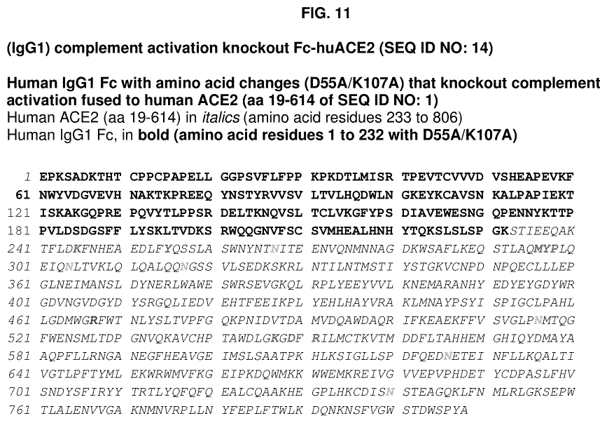

In one embodiment of the invention, recombinant fusion proteins will have ACE2 at the amino terminal end and a portion of an immunoglobulin, for example an Fc, at the carboxyl terminal end of the fusion protein yielding an ACE2-Fc molecule. In another embodiment of the invention recombinant fusion proteins will have ACE2 at the carboxy terminal end and a portion of an immunoglobulin, for example an Fc, at the amino terminal end of the fusion protein yielding a Fc-ACE2 orientation. Because the majority of the residues involved in binding of ACE2 to the SARS-CoV-2 spike protein, are at the amino-terminal end of the ACE2 amino acid sequence, the former of the above two orientations is preferred for the receptor-Fc and modified receptor-Fc fusion proteins according to the invention. In an additional embodiment of the invention a linker comprised of four or more amino acid residues is covalently linked by peptide bonds between the ACE2 or modified ACE2 amino acid sequence and the immunoglobulin Fc sequence. For example, in FIGS. 2, 3, 4, 8, 10, and 13 a linker may be covalently bound between residue 597 the end of the ACE2 or modified ACE2 sequence and residue 598, the first residue of the Fc sequence. In FIGS. 5, 6, 7, 9,11 and 14 a linker may be covalently bound between the last residue of the Fc sequence which is depicted in bold letters in the figures and the first residue of the ACE2 or modified ACE2 sequence, which begins with the amino acid residue sequence stieeqaktf.

Hence, compositions according to the invention include constructs comprise a functional ACE2 amino acid sequence which may comprise either the entire ACE2 protein sequence (805 amino acid residues) or just the extracellular domain or soluble receptor portion of the ACE2 protein required for binding to SARS-CoV-2 a 721 amino acid length sequence within the ACE2 sequence spanning amino acids 19-740 of the FIG. 1, or a shorter sequence of the extra cellular domain of 596 amino acid residues spanning residues 19-614 in FIG. 1), or just a smaller sub-region of soluble receptor of the ACE2 protein required for binding to SARS-CoV-2 (fewer than 596 amino acid residues).

In one preferred embodiment, the composition according to the invention comprises the shorter sequence of the extra cellular domain of 596 amino acid residues (spanning residues 19-614 in FIG. 1) fused to a moiety that extends its half-life. Both the entire ACE2 protein sequence (805 amino acid residues) and the extracellular or soluble receptor portion of the ACE2 protein required for binding to SARS-CoV-2 which includes the 721 amino acid length sequence within the ACE2 sequence spanning amino acids 19-740 of the FIG. 1, have potential cleavage sites if they are expressed in plants. Such cleavage sites suffer from site specific proteolytic degradation when found in monoclonal antibodies when expressed in tobacco plants. (Hehle et al., Plant Biotechnology Journal (2015) 13, pp. 235-245). Cleavage of the ACE2 receptor protein in the amino acid sequence SLKSA, KKNKA or IDISKG would be expected in fusions that comprise either the full length (805 amino acids) ACE2 protein sequence protein and complete extracellular or soluble ACE2 protein sequence (721 amino acids). All three of these proteolytically labile sequences are found after the amino acid residue at 614 in the human ACE2 protein sequence of SEQ ID NO: 1 (shown in FIG. 1). Specifically, within the amino acid sequence of SEQ ID NO: 1 the proteolytic site SLKSL spans residues 623 to 627, the proteolytic site KKNKA spans residues 770 to 774 and the proteolytic site IDISKG spans residues 784 to 788.

In another preferred embodiment, wherein the moiety is a portion of the immunoglobulin heavy chain preferably an Fc or hinge and Fc, the composition will be approximately the size of a dimeric IgG, IgA or an IgM. In some embodiments a linker may be covalently bound between the region comprising the moiety comprising an Fc and the functional ACE2 amino acid sequence, and if shorter linkers such as (Gly₃Ser)₃ or (Gly₄Ser)₃ are employed the composition will still have the approximate size of a dimeric IgG, IgA or an IgM. When such linkers are employed the functional ACE2 amino acid sequence may terminate at residue 615. In the absence of such linkers, the functional ACE2 amino acid sequence may terminate at amino acid residue 614. When the fusion protein forms homo-dimers, as a result of dimerization of the Fc region, the two ACE2:SARS-CoV-2 binding sites will be separated by about the same distance as the combining sites on normal dimeric antibodies. Because the spikes on a typical coronavirus virion are situated about 15 nM apart (Neuman et al. 2006), in a preferred embodiment, the IgA1 fusion may be able to bind two spikes simultaneously. In another preferred embodiment Fc of IgA2 and Fc of IgG fusions containing the entire ACE2 extracellular domain may also achieve improved neutralization.

In addition to the potential for superior virus neutralization, a fusion of ACE2 to the Fc of IgG1 has two additional advantages: as a therapeutic an increased circulating half-life due to the ability of Fc to bind to the neonatal Fc receptor (FcRn) for recycling (Rath et al. 2013) and a simplified purification using affinity chromatography, for example protein A affinity chromatography. Furthermore, a fusion of ACE2 to the Fc of IgA1 has the additional advantage in purification using affinity ligands designed for purification of human IgA.

Furthermore, because ACE2-Fc has a longer circulating half-life than soluble ACE2, the ACE2-Fc or modified ACE2-Fc would be employed in a prophylactic mode (either pre- or post-exposure), providing long-lasting protection upon administration (e.g. for first responders).

Because ACE2-Fc or modified ACE2-Fc does not rely on an active immune response, it could protect immune-compromised patients who may not respond to a vaccine.

In addition because there is no obvious mechanism for soluble ACE2 administered parenterally by injection (intravenously, intramuscularly or intraperitoneally) to move to airway mucosal surfaces, the primary site of SARS-CoV-2 infection, ACE2-Fc or modified ACE2-Fc would have the ability to move from circulation to lung mucosal surfaces by trans-epithelial transport via the neonatal Fc receptor (FcRn).Of clinical importance to patients with COVID-19 respiratory symptoms, ACE2-Fc or modified ACE2-Fc can be administered directly into the upper respiratory tract as nasal drops or deeper into the lungs via a nebulizing inhaler. There it could bind to virus particles, trapping them in the mucus secretions overlying the lung epithelium, preventing them from infecting naïve cells and promoting their elimination by ciliary action. ACE2-Fc or modified ACE2-Fc may also promote cellular phagocytosis of SARS-CoV-2 by alveolar macrophages.

Although application of the receptor-Fc fusion approach to treatment of COVID-19 is novel, the approach has previously been applied to develop therapeutics for other pathogens, including HIV, Hepatitis A virus, Pneumocystis carinii and coxsackievirus (Rapaka et al. 2007; Silberstein et al. 2003; Ward et al. 1991; Lim et al. 2006).

Differences in the identity of the ACE2-RBD contact amino acids among species have been identified and present an opportunity to modify the affinity of binding of the various ACE2-Fcs. Although the SARS-CoV-2 S1 glycoprotein binds to human ACE2, it may be better adapted to bind to the ACE2 of its animal host. A fusion protein based on the binding surface of the animal ACE2 might be more or less potent at neutralizing SARS-CoV-2 infection. For that reason, single amino acid changes are made at specific positions in the human ACE2 of the fusion proteins, based on conjectures of the potential SARS-CoV-2 animal host. The animal host, which has not yet definitively been identified, is believed to be a pangolin species from Malaysia and China, turtles, snakes, or bats, based on sequence similarities between coronaviruses isolated from these animals and SARS-CoV-2. Thus, the invention includes functional human ACE2 sequences altered from the native human sequence that have a higher binding affinity for the SARS-CoV-2 spike protein and hence SARS-CoV-2 itself. Thus, a preferred embodiment of the invention includes altered soluble human ACE2 having a higher binding affinity to SARS-CoV-2 than native soluble human ACE2. Such high binding affinity-altered soluble human ACE2s of the invention may alone bind to and neutralize SARS-CoV-2. Furthermore, high binding affinity-altered soluble human ACE2s and the nucleic acid sequences that encode them herein disclosed are valuable as intermediates in the recombinant production of ACE2-Fc fusions.

SARS-CoV-2 Receptor Biding Domain (RBD) amino acid residues (the domain that binds to ACE2) have no contact with the region of ACE2 protein that has enzymatic activity. The enzymatic activity of ACE2 is retained by the huACE2 when linked to an Fc immunoglobulin in a fusion protein. In order not to introduce into subjects in need of treatment for COVID-19 an ACE2-derived therapeutic with additional Angiotensin II converting activity, it may be useful to eliminate the enzymatic activation capability of a therapeutic ACE2-Fc fusion, with as few amino acid changes as possible.

To eliminate enzymatic activity from ACE2, the amino acid residue R273 of SEQ ID NO: 1 is changed to K, keeping the charge on the molecule the same. This single amino acid modification will eliminate the ACE2 enzymatic activity as shown by the enzymatic assay (Guy et al. 2005; Vickers et al. 2002), yet should have no effect on folding of the rest of the ACE2 peptide sequence domain and thus the binding ability of S1 to bind to the enzymatically inactivated ACE2 receptor for SARS-CoV-2.

Alternatively, subjects in need of treatment for COVID-19 may also have diminished ACE2 activity resulting from the infection, and it may be clinically beneficial to retain ACE2 activity in the ACE2-Fc or modified ACE2-Fc of the invention. ACE2 is a part of the renin-angiotensin system (RAS), playing a key role in maintaining blood pressure homeostasis, as well as fluid and salt balance. Recent studies indicate that the RAS plays a critical role in acute lung diseases, especially acute respiratory distress syndrome (ARDS). In the lung, ACE2 protects against acute lung injury in several animal models of ARDS. Thus, the RAS appears to play a critical role in the pathogenesis of acute lung injury. Indeed, increasing ACE2 activity has been proposed as a novel approach for the treatment of acute lung failure in several diseases (38). ACE2-Fc or modified ACE2-Fc could be employed as a means of introducing additional ACE2 activity to the lung in the form of ACE2-Fc when administered to COVID-19 patients. ACE2-Fc or ACE2-Fc variants with R273 unchanged can be used to increase ACE2 activity in the lung when such increased enzymatic activity proves beneficial.

The more severe outcomes of SARS-CoV2 infection may be attributable to antibody dependent enhancement (ADE) due to prior exposure to other coronaviruses (40). ADE modulates the immune response and can elicit sustained inflammation, lymphopenia, and/or cytokine storm, one or all of which have been documented in severe COVID-19 cases and deaths. Passive immunization with anti-S antibodies induced acute lung injury in Chinese rhesus monkeys (Macaca mulatta) infected with SARS-CoV (41). A recent study using MERS-CoV and SARS-CoV showed that ADE of coronaviruses is mediated by neutralizing mAbs that target the RBD of coronavirus spikes. These mAbs blocked SARS-CoV or MERS-CoV pseudovirus entry into cells expressing the cognate receptor (ACE2 and DPP4, respectively) but enhanced coronavirus uptake into cells expressing the low-affinity FcγR (CD32a). The ADE effect was dose dependent, only occurring between 100 and 500 ng/ml (42).

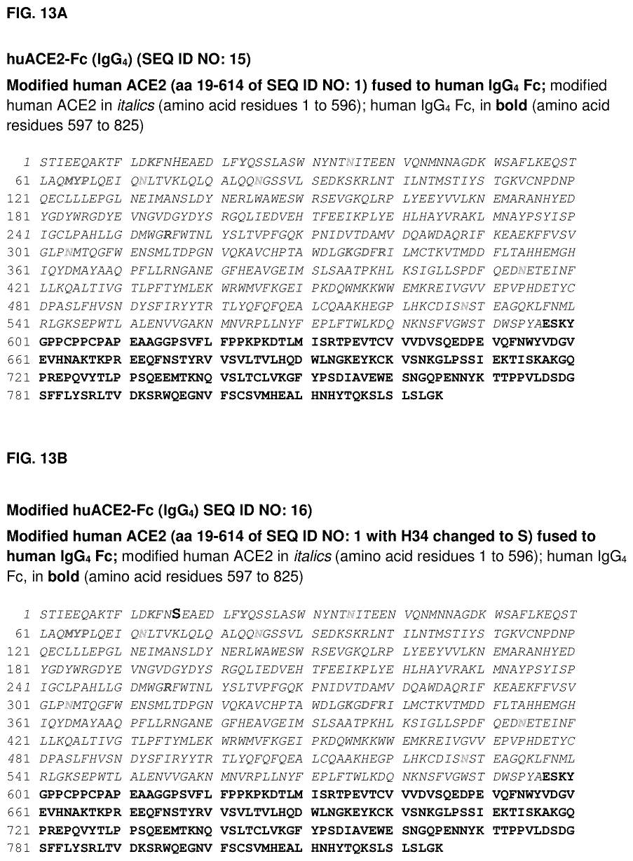

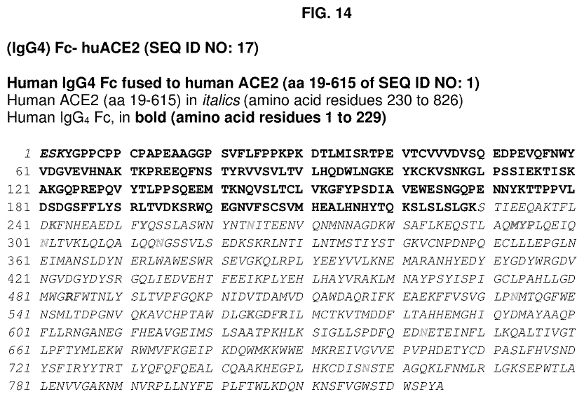

ADE can be reduced by using Fc from IgG4, which does not bind to FcγRIIa and has significantly reduced effector function. Thus among the ACE2-Fc and modified ACE2-Fc provided according to the invention are variants having a modified IgG4 Fc that was used in Dulaglutide. In this modified form of IgG₄ Fc, two selected positions have been mutated F234 to A and L235 to A according to the Kabat amino acid sequence numbering which corresponds to position 613 and 614 of the IgG₄ of FIG. 13A and position 16 and 17 of the IgG₄ Fc of FIG. 14, to reduce interaction with high-affinity Fc receptors. In addition, a modification of S228 to P according to the Kabat amino acid sequence numbering, which corresponds to position 607 of the IgG₄ of FIG. 13A and position 10 of the IgG₄ sequence of FIG. 14, eliminates Fab arm exchange between IgG4 half-molecules (45).

Modification of the Fc Sequence with and without KDEL

The ACE2-Fc variants of the invention may include the ER retention signal KDEL, appended to the Fc C-terminus. The use of the ER retention signal KDEL results in the high-mannose form for the proteins' N-glycans (Petruccelli et al. 2006). Alternatively, the ACE2-Fc variants of the invention may be produced without ER retention signal KDEL. The N-glycans of the ACE2-Fc variants lacking the ER retention signal KDEL will be of the complex type on both ACE2 and Fc regions of the protein. Antibodies with high-mannose glycans are cleared from circulation more rapidly than those with complex type glycans in mice (Kanda et al. 2007) and humans (Goetze et al. 2011); ACE2-Fc variants with complex N-glycans should therefore possess improved pharmacodynamic characteristics.

In one embodiment of the invention, the ACE2-Fc-fusions of the invention may be expressed in eukaryotic cells, tissues, organs or organisms, including fungal, insect, plant cell or mammalian cell culture according to known cell culture conditions. In a preferred embodiment the ACE2-Fc-fusions according to the invention are made in intact plant cells. Such plants may be transformed so that the nucleic acid sequences encoding the ACE2-Fc-fusion are stably incorporated into the plant genome and expressed in the cells and tissues of the intact plant and are transmitted from one generation to the next through the development of seed incorporating the nucleic acid sequences encoding the ACE2-Fc-fusion.

In another preferred embodiment of the invention the ACE2-Fc-fusions according to the invention are made in intact plants that have been transfected with Agrobacterium tumefaciens wherein the Ti plasmid has been engineered to contain the nucleic acid sequences encoding the ACE2-Fc-fusion protein which are transiently expressed by the cells and tissues of the intact plant. According to this method of production in plants, the open reading frames encoding a ACE2-Fc fusion described above is cloned into the plant expression vector pTRAk with suitable promoters and expression control sequences and the resulting vectors are transformed into Agrobacterium tumefaciens. The Agrobacterium strains will be used for transient transformation of Nicotiana benthamiana plants, with the recombinant protein expressed in plant cells. The fusion protein will be purified from extracts of plant tissue using standard chromatographic procedures, including, if the ACE2-Fc fusion comprises an IgG heavy chain, Protein A affinity chromatography or if the ACE2-Fc fusion comprises an IgA heavy chain, other affinity reagents including for example Protein G, CaptureSelect IgA Affinity Matrix (Life Technologies) and the like.

Proper N-glycosylation of the Fc may be important for in vivo viral neutralization. Accordingly, it is preferred to produce the ACE2-Fc fusion proteins with N-glycans as similar to typical mammalian N-glycans as possible using an N. benthamiana line in which the endogenous β1,2-xylosyltransferase (XylT) and α1,3-fucosyltransferase (FucT) genes have been down-regulated by RNA interference. Such strains are produced as described in (Strasser et al. 2008). Glycoproteins produced in this line contain almost homogeneous N-glycan species without detectable plant-specific β1,2-xylose and α1,3-fucose residues. The expression of the XylT gene and FucT gene may be down regulated or eliminated by methods other than RNA interference, including by modification using the CRISPR/Cas system to alter the sequence of the genes encoding one or both proteins. Additionally, to ensure uniform addition of terminal β1,4-Gal residues to N-glycans (Strasser et al. 2009), it is additionally preferred to co-infiltrate this N. benthamiana with a binary vector that encodes a modified human β1,4-galactosyl-transferase (ST-Gaff) to “humanize” plant-made N-glycans.

There is another reason why appropriate ACE2-Fc N-glycosylation may be important. ACE2 with complex N-glycans similar to typical human N-glycans may have increased affinity for SARS-COV-2 S1 spike protein. Amino acid residues of ACE2 having N-glycans are found in the amino acid sequence of SEQ ID NO: 1 at residues 53, 90, 103, 322, 432 and 546.

While the forgoing description is applicable to production of recombinant ACE2 moieties such as ACE2-Fc, Fc-ACE2 with or without modifications to the ACE2 amino acid sequence in plant cells and plants, other systems for production of the ACE2-Fc or modified ACE2-Fc as recombinant molecules can be used.

The disclosure provides polynucleotides comprising nucleic acid sequences that encode ACE2 moieties with or without modifications to the ACE2 amino acid sequence described herein. A polynucleotide can comprise a DNA or RNA nucleic acid sequence that encodes ACE2 or improved variants thereof.

The disclosure further provides constructs (which may also be called expression or cloning constructs) comprising nucleic acid sequences that encode ACE2 moieties described herein. Suitable constructs include, but are not limited to, plasmids, including Ti Plasmids, cosmids, bacterial artificial chromosomes, yeast artificial chromosomes, lambda phages (e.g., those with lysogeny genes deleted), and viruses. A construct can be present in a cell episomally or integrated into a chromosome (either way the construct remains and is still a construct, a plasmid and a vector).

Various construct systems can be employed in addition to those specifically exemplified herein. One class of constructs utilize DNA elements of bacteria that are capable of transfecting plants such as Agrobacterium tumafaciens, while another class of constructs use RNA elements of plant viruses exemplified by Tobacco Mosaic Virus, Cauliflower Mosaic Virus. Yet another class of constructs utilize DNA elements derived from animal viruses such as adenovirus, baculovirus, bovine papilloma virus, polyoma virus, SV40 virus, vaccinia virus, and retroviruses (e.g., MMTV, MOMLV and Rous sarcoma virus). Another class of constructs utilize RNA elements derived from RNA viruses such as eastern equine encephalitis virus, flaviviruses and Semliki Forest virus.

A construct can comprise various other elements for optimal expression of mRNA in addition to a nucleic acid sequence that encodes, e.g., the ACE2 moieties or improved variants thereof. For example, a construct can contain a transcriptional promoter, a promoter plus an operator, an enhancer, an open reading frame with or without intron(s) or/and exon(s), a termination signal, a splice signal, a secretion signal sequence or a selectable marker (e.g, a gene conferring resistance to an antibiotic or cytotoxic agent), or any combination or all thereof.

The disclosure also provides host cells comprising or expressing constructs that encode ACE2 fusion proteins described herein. Suitable host cells include, but are not limited to, eukaryotic cells, including plant cells in intact plants, plant callus culture, or plant cell culture on surfaces or in suspension such as those derived from the genus Nicotiana such as N. tabaccum and N. benthamiana, and genus Daucus such as D. carota, mammalian cells (e.g., BHK, CHO, COS, HEK293, HeLa, MDCKII and Vero cells), insect cells (e.g., Sf9 cells), yeast cells and bacterial cells (e.g., E coli cells). The host cell can be a mammalian cell (e.g., a CHO cell or a HEK293 cell).

A host cell can comprise or express a construct that encodes an ACE2 moiety or improved variants thereof. A host cell can comprise or express a single construct that encodes the ACE2 variant.

A construct can be transfected or introduced into a host cell by any method known in the art. Transfection agents and methods include without limitation calcium phosphate, cationic polymers (e.g., DEAE-dextran and polyethylenimine), dendrimers, fugene, cationic liposomes, electroporation, sonoporation, cell squeezing, gene gun, bacterial transfection as with A. tumanfaciens, viral transfection and retroviral transduction.

Methods and conditions for culturing transfected host cells and recovering the recombinantly produced ACE2 moiety are known in the art, and may be varied or optimized depending on, e.g., the particular expression vector or/and host cell employed. The ACE2 moiety or improved variants thereof can be recombinantly produced. ACE2 may be optionally fused with a protracting moiety, and recombinantly produced.

In another embodiment of the invention, the ACE2-Fc or modified ACE2-Fc may be delivered to the body by various routes including parenteral, preferably intravenous, intraarterial and intraperitoneal, or by mucosal administration. FcRn mediates the endocytic salvage pathway responsible for the long circulating half-life of IgGs (Goebl et al. 2008) and also mediates bi-directional IgG transcytosis across mucosal epithelial cells in a variety of adult human tissues. FcRn is expressed in the mucosal epithelial cells lining the conducting airways (the trachea and bronchioles) (Spiekermann et al. 2002) and is responsible for the high IgG concentration in airway surface liquid (up to 17% of total protein) (Goldblum and Garofolo 2004; Hand and Cantey 1974). Bi-directional IgG transport between the blood and the lumen of the airways is facilitated because the epithelium lies on top of the basement membrane, which lies directly above the highly vascularized lamina propria. For this reason parenterally administered ACE2-Fc will be delivered to airway mucosal surfaces, which is the site of SARS-CoV-2 infection (Tao et al. 2013).

Pharmaceutical Compositions

Additional embodiments of the disclosed invention relate to pharmaceutical compositions comprising a functional ACE2-Moiety, for example a functional ACE2-Fc or a pharmaceutically acceptable salt, solvate or hydrate thereof, and one or more pharmaceutically acceptable excipients or carriers. The compositions can optionally contain an additional therapeutic agent. In general, a pharmaceutical composition contains a therapeutically effective amount of an ACE2-Moiety or a fragment thereof, one or more pharmaceutically acceptable excipients or carriers and optionally a therapeutically effective amount of an additional therapeutic agent, and is formulated for administration to a subject for therapeutic use.

Pharmaceutical compositions generally are prepared according to current good manufacturing practice (GMP), as recommended or required by, e.g., the Federal Food, Drug, and Cosmetic Act § 501(a)(2)(B) and the International Conference on Harmonisation Q7 Guideline.

Pharmaceutical compositions/formulations can be prepared in sterile form. For example, pharmaceutical compositions/formulations for parenteral administration by injection or infusion generally are sterile. Sterile pharmaceutical compositions/formulations are compounded or manufactured according to pharmaceutical-grade sterilization standards known to those of skill in the art, such as those disclosed in or required by the United States Pharmacopeia Chapters 797, 1072 and 1211, and 21 Code of Federal Regulations 211.

Pharmaceutically acceptable excipients and carriers include pharmaceutically acceptable substances, materials and vehicles. Non-limiting examples of types of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, antioxidants, preservatives, antimicrobial agents, antibacterial agents, antifungal agents, chelating agents, adjuvants, sweetening agents, flavoring agents, coloring agents, encapsulating materials and coating materials. The use of such excipients in pharmaceutical formulations is known in the art. For example, conventional vehicles and carriers include without limitation oils (e.g., vegetable oils such as olive oil and sesame oil), aqueous solvents (e.g., saline, buffered saline (e.g., phosphate-buffered saline [PBS]) and isotonic solutions (e.g., Ringer's solution)), and organic solvents (e.g., dimethyl sulfoxide [DMSO] and alcohols [e.g., ethanol, glycerol and propylene glycol]). Except insofar as any conventional excipient or carrier is incompatible with a functional ACE2-Moiety or a fragment thereof, the disclosure encompasses the use of conventional excipients and carriers in formulations containing a functional ACE2-Moiety or a fragment thereof. See, e.g., Remington: The Science and Practice of Pharmacy, 21st Ed., Lippincott Williams & Wilkins (Philadelphia, Pa.) (2005); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press (Boca Raton, Fla.) (2004).

Appropriate formulation can depend on various factors, such as the route of administration chosen. Potential routes of administration of a pharmaceutical composition comprising a Functional ACE2-Moiety or a fragment thereof include without limitation oral, parenteral (including intradermal, subcutaneous, intramuscular, intravascular, intravenous, intraarterial, intraperitoneal, intramedullary, intrathecal and topical), intracavitary, and topical (including dermal/percutaneous, transdermal, mucosal, transmucosal, intranasal [e.g., by nasal spray, drop or nebulizer], intraocular [e.g., by eye drop], pulmonary [e.g., by oral or nasal inhalation for example using a nebulizer], buccal, sublingual, rectal [e.g., by suppository], and vaginal [e.g., by suppository]). Topical formulations can be designed to produce a local or systemic therapeutic effect. In certain embodiments, a functional ACE2-Moiety or a fragment thereof is administered parenterally (e.g., intravenously, subcutaneously, intramuscularly or intraperitoneally) by injection (e.g., as a bolus) or by infusion over a period of time.

Excipients and carriers that can be used to prepare parenteral formulations include without limitation solvents (e.g., aqueous solvents such as water, saline, physiological saline, buffered saline [e.g., phosphate-buffered saline], balanced salt solutions [e.g., Ringer's BSS] and aqueous dextrose solutions), isotonic/iso-osmotic agents (e.g., salts [e.g., NaCl, KCl and CaCl2)] and sugars [e.g., sucrose]), buffering agents and pH adjusters (e.g., sodium dihydrogen phosphate [monobasic sodium phosphate]/disodium hydrogen phosphate [dibasic sodium phosphate], citric acid/sodium citrate and L-histidine/L-histidine HCl), and emulsifiers (e.g., non-ionic surfactants such as polysorbates [e.g., polysorbate 20 and 80] and poloxamers [e.g., poloxamer 188]). Protein formulations and delivery systems are discussed in, e.g., A. J. Banga, Therapeutic Peptides and Proteins: Formulation, Processing, and Delivery Systems, 3rd Ed., CRC Press (Boca Raton, Fla.) (2015).

The excipients can optionally include one or more substances that increase protein stability, increase protein solubility, inhibit protein aggregation or reduce solution viscosity, or any combination or all thereof. Examples of such substances include without limitation hydrophilic amino acids (e.g., arginine and histidine), polyols (e.g., myo-inositol, mannitol and sorbitol), saccharides (e.g., glucose (including D-glucose [dextrose]), lactose, sucrose and trehalose), osmolytes (e.g., trehalose, taurine, amino acids [e.g., glycine, sarcosine, alanine, proline, serine, β-alanine and γ-aminobutyric acid], and betaines [e.g., trimethylglycine and trimethylamine N-oxide]), and non-ionic surfactants (e.g., alkyl polyglycosides, ProTek® alkylsaccarides (e.g., a monosaccharide [e.g., glucose] or a disaccharide [e.g., maltose or sucrose] coupled to a long-chain fatty acid or a corresponding long-chain alcohol), and polypropylene glycol/polyethylene glycol block co-polymers (e.g., poloxamers [e.g., Pluronic™ F-68], and Genapol® PF-10 and variants thereof)). Because such substances increase protein solubility, they can be used to increase protein concentration in a formulation. Higher protein concentration in a formulation is particularly advantageous for subcutaneous administration, which has a limited volume of bolus administration (e.g., about 1.5 mL). In addition, such substances can be used to stabilize proteins during the preparation, storage and reconstitution of lyophilized proteins.

The ACE2-Fc according to the invention may be provided as an aerosol, produced by a nebulizer or inhaler. Such an aerosol may be administered by inhalation to subjects who are infected with SARS-CoV-2 who are asymptomatic, or through a ventilator in acutely ill patients, either of which have confirmed SARS-CoV-2 infections by PCR or other diagnostic testing. Inhalation of anti-infectious mAbs in models of pneumonia using Pseudomonas aeruginosa or influenza virus conferred higher protection and greater therapeutic response, respectively, compared to parenteral route administration. Tiziana Life Sciences (London, UK) recently announced an investigational new technology to treat COVID-19 infections, consisting of direct delivery of anti-IL-6 receptor (anti-IL-6R) mAbs into the lungs using a handheld inhaler or nebulizer (52).

For viral respiratory disease there are advantages for aerosol administration in treating pulmonary viral infections; however, there are significant challenges to this modality. Nebulization—the process of converting an aqueous liquid into an aerosol—exposes proteins to stressful conditions by generating a huge air-liquid interface and, in some cases, high temperatures and/or shear forces. These conditions may cause protein unfolding, aggregation, oxidation, deamidation or glycation, which may lead to changes in biological activity and safety concerns. Comparative studies of nebulizers have identified a number of trends (70, 71). Vibrating-mesh nebulizers are the type most frequently used for therapeutic protein delivery in humans (86% of the cases in which the nebulizer technology is disclosed) (72). Multiple studies have shown vibrating mesh nebulizers are better than other nebulizer types at maintaining IgG binding affinity and causing minimal aggregation (71, 73-76). Inhalation of anti-infectious mAbs in models of pneumonia using Pseudomonas aeruginosa or influenza virus conferred higher protection and greater therapeutic response, respectively, compared to parenteral route administration (50, 51).

The PARI eFlow (PARI Respiratory Equipment Inc, Midlothian, Va.) is a popular mesh nebulizer. Dornase alpha (Pulmozyme®, oral inhalation), a mucolytic agent for patients with CF (29 kDa) is delivered as an aerosol using a PARI nebulizer. Two other pulmonary aerosol protein drugs in clinical trials, GM-CSF (clinicaltrials.gov: NCT03597347) and alpha-1-antitrypsin (clinicaltrials.gov: NCT02001688), are administered using the PARI eFlow nebulizer system.

For example, a formulation for a nebulizer that can deliver ACE2-Fc in aerosol particles of 1-5 μm diameter (to reach the alveoli) is desirable. Initially ACE2-Fc can be formulated in an appropriate aqueous buffer, for example phosphate-buffered saline (PBS) at varying concentrations (ranging from 1 μg/ml up to 30 mg/ml, preferably between 10 μg/ml and 1 mg/ml, or between 100 μg/ml and 1 to 10 mg/ml) will be loaded into and released through the nebulizer. The effect of nebulization on protein integrity can be determined by SDS-PAGE and on aggregation by analytical SEC. Aa variety of excipients already known to stabilize proteins and/or improve aerosols, including surfactants (e.g. polysorbates), polyethylene glycols, sugars, polyols and amino acids may be incorporated into the formulation.

For parenteral (e.g., intravenous, subcutaneous or intramuscular) administration, a sterile solution or suspension of a functional ACE2-Moiety in an aqueous solvent containing one or more excipients can be prepared beforehand and can be provided in, e.g., a pre-filled syringe. Alternatively, a functional ACE2-Moiety can be dissolved or suspended in an aqueous solvent that can optionally contain one or more excipients prior to lyophilization (freeze-drying). Shortly prior to parenteral administration, the lyophilized functionalACE2-Moiety stored in a suitable container (e.g., a vial) can be reconstituted with, e.g., sterile water that can optionally contain one or more excipients. If the functional ACE2-Moiety is to be administered by infusion (e.g., intravenously), the solution or suspension of the reconstituted ACE2 moiety can be added to and diluted in an infusion bag containing, e.g., sterile saline (e.g., about 0.9% NaCl). In the foregoing paragraph, the Moiety is preferably a human immunoglobulin Fc.

Excipients that enhance transmucosal penetration of smaller proteins include without limitation cyclodextrins, alky saccharides (e.g., alkyl glycosides and alkyl maltosides [e.g., tetradecylmaltoside]), and bile acids (e.g., cholic acid, glycocholic acid, taurocholic acid, deoxycholic acid, glycodeoxycholic acid, chenodeoxycholic acid and dehydrocholic acid).

Excipients that enhance transepithelial or transdermal penetration of smaller proteins include without limitation chemical penetration enhancers (CPEs, including fatty acids [e.g., oleic acid]), cell-penetrating peptides (CPPs, including arginine-rich CPPs [e.g., polyarginines such as R6-R11 (e.g., R6 and R9) and TAT-related CPPs such as TAT(49-57)] and amphipathic CPPs [e.g., Pep-1 and penetratin]), and skin-penetrating peptides (SPPs, such as the skin-penetrating and cell-entering peptide). Transdermal penetration of smaller proteins can be further enhanced by use of a physical enhancement technique, such as iontophoresis, cavitational or non-cavitational ultrasound, electroporation, thermal ablation, radio frequency, microdermabrasion, microneedles or jet injection. US 2007/0269379 provides an extensive list of CPEs. F. Milletti, Drug Discov. Today, 17:850-860 (2012) is a review of CPPs. R. Ruan et al., Ther. Deliv., 7:89-100 (2016) discuss CPPs and SPPs for transdermal delivery of macromolecules, and M. Prausnitz and R. Langer, Nat. Biotechnol., 26:1261-1268 (2008) discuss a variety of transdermal drug-delivery methods.

A functional ACE2-Moiety can be delivered from a sustained-release composition. As used herein, the term “sustained-release composition” encompasses sustained-release, prolonged-release, extended-release, slow-release and controlled-release compositions, systems and devices. Protein delivery systems are discussed in, e.g., Banga (supra). A sustained-release composition can deliver a therapeutically effective amount of a functional ACE2-Moiety over a prolonged time period. In some embodiments, a sustained-release composition delivers a functional ACE2-Moiety over a period of at least about 3 days, 1 week, 2 weeks, 3 weeks, 1 month (4 weeks), 6 weeks, 2 months, 3 months or longer. A sustained-release composition can be administered, e.g., parenterally (e.g., intravenously, subcutaneously or intramuscularly). In the foregoing paragraph, the Moiety is preferably a human immunoglobulin Fc.

A sustained-release composition of a protein can be in the form of, e.g., a particulate system, a lipid or oily composition, or an implant. Particulate systems include without limitation nanoparticles, nanospheres, nanocapsules, microparticles, microspheres and microcapsules. Nanoparticulate systems generally have a diameter or an equivalent dimension smaller than about 1 μm. In certain embodiments, a nanoparticle, nanosphere or nanocapsule has a diameter or an equivalent dimension of no more than about 500, 400 or 300 nm, or no more than about 200, 150 or 100 nm. In some embodiments, a microparticle, microsphere or microcapsule has a diameter or an equivalent dimension of about 1-200, 100-200 or 50-150 μm, or about 1-100, 1-50 or 50-100 μm. A nano- or micro-capsule typically contains the therapeutic agent in the central core, while the therapeutic agent typically is dispersed throughout a nano- or micro-particle or sphere. In certain embodiments, a nanoparticulate system is administered intravenously, while a microparticulate system is administered subcutaneously or intramuscularly.

In some embodiments, a sustained-release particulate system or implant is made of a biodegradable polymer or/and a hydrogel. In certain embodiments, the biodegradable polymer comprises lactic acid or/and glycolic acid [e.g., an L-lactic acid-based copolymer, such as poly(L-lactide-co-glycolide) or poly(L-lactic acid-co-D,L-2-hydroxyoctanoic acid)]. Non-limiting examples of polymers of which a hydrogel can be composed include polyvinyl alcohol, acrylate polymers (e.g., sodium polyacrylate), and other homopolymers and copolymers having a relatively large number of hydrophilic groups (e.g., hydroxyl or/and carboxylate groups). The biodegradable polymer of the particulate system or implant can be selected so that the polymer substantially or completely degrades around the time the period of treatment is expected to end, and so that the byproducts of the polymer's degradation, like the polymer, are biocompatible.

Alternatively, a sustained-release composition of a protein can be composed of a non-biodegradable polymer. Examples of non-biodegradable polymers include without limitation poloxamers (e.g., poloxamer 407). Sustained-release compositions of a protein can be composed of other natural or synthetic substances or materials, such as hydroxyapatite.

Sustained-release lipid or oily compositions of a protein can be in the form of, e.g., liposomes, micelles (e.g., those composed of biodegradable natural or/and synthetic polymers, such as lactosomes), and emulsions in an oil. A sustained-release composition can be formulated or designed as a depot, which can be injected or implanted, e.g., subcutaneously or intramuscularly. A depot can be in the form of, e.g., a polymeric particulate system, a polymeric implant, or a lipid or oily composition. A depot formulation can comprise a mixture of a protein and, e.g., a biodegradable polymer [e.g., poly(lactide-co-glycolide)] or a semi-biodegradable polymer (e.g., a block copolymer of lactic acid and PEG) in a biocompatible solvent system, whether or not such a mixture forms a particulate system or implant.

A pharmaceutical composition can be presented in unit dosage form as a single dose wherein all active and inactive ingredients are combined in a suitable system, and components do not need to be mixed to form the composition to be administered. The unit dosage form generally contains an effective dose of the therapeutic agent. A representative example of a unit dosage form is a single-use pen comprising a pre-filled syringe, a needle and a needle cover for parenteral (e.g., intravenous, subcutaneous or intramuscular) injection of the therapeutic agent. An alternative unit dosage form for administration to the respiratory tract is a single-use or multiple use pre-filled nebulization device suitable for delivery of a nebulized protein such as ACE-Fc by oral or nasal inhalation.

Alternatively, a pharmaceutical composition can be presented as a kit in which the therapeutic agent, excipients and carriers (e.g., solvents) are provided in two or more separate containers (e.g., ampules, vials, tubes, bottles, nebulizer or syringes) and need to be combined to form the composition to be administered. The kit can contain instructions for storing, preparing and administering the composition (e.g., a solution to be injected intravenously or subcutaneously).

A kit can contain all active and inactive ingredients in unit dosage form or the active ingredient and inactive ingredients in two or more separate containers and can contain instructions for administering or using the pharmaceutical composition to treat a medical condition.

In some embodiments, a kit contains a functional ACE2-Moiety or a pharmaceutical composition comprising the same, and instructions for administering or using the Functional ACE2-Moiety or the pharmaceutical composition comprising the same to treat an antibody-associated condition.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Transient Expression of ACE2-Fc (IgG1) Fusion Proteins in N. benthamiana

Briefly, DNA sequences encoding the full-length human ACE2 extracellular domain (805 amino acids of SEQ ID NO: 1) or the soluble human ACE2 domain sequence (596 amino acids corresponding to residues 19-614 of SEQ ID NO: 1) or a Modified ACE2 sequence as defined above is PCR-amplified and then cloned into the pTRAkc plant binary vector (Maclean et al. 2007) in frame with an IgG1 Fc sequence optimized for expression in planta. Recombinant A. tumefaciens strains (GV3101::pMP90RK) carrying these expression vectors are used to transiently express the ACE2-Fc, soluble ACE2-Fc, or Modified ACE2-Fc in whole N. benthamiana plants following vacuum-assisted agroinfiltration using known methods (Kapila et al. 1997; Vaquero et al. 1999). Co-infiltration of an additional A. tumefaciens strain (GV3101::pMP90RK) carrying the p19 silencing suppressor from tomato bushy stunt virus is used to prevent post-transcriptional gene silencing and hence enhance expression levels (Voinnet et al. 2003). The transfected plants are harvested, and plant juice is extracted by grinding in a Waring blender. The juice is separated by filtration and the protein is purified by Protein A chromatography. Reduced and non-reduced samples are separated by SDS-PAGE and stained with Coomassie dye (a) or probed with anti-ACE2 antibodies (b). Monomer (reduced) and dimer bands are detected at the expected positions.

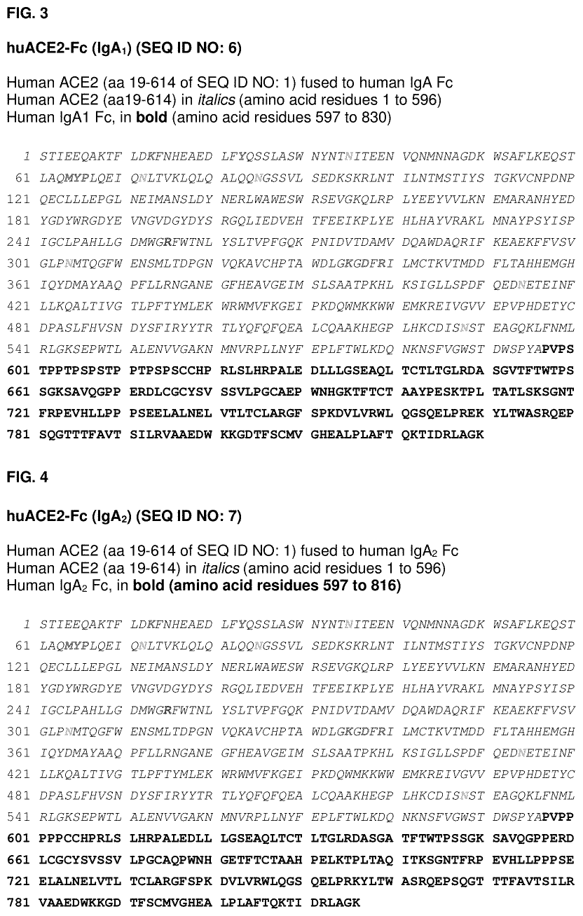

In greater detail, expression vectors are produced as follows. DNA sequences encoding the full-length extracellular domain human ACE2 of SEQ ID NO: 1 or the soluble human ACE2 sequence (720 amino acids corresponding to residues 19 to 740 of SEQ ID NO: 1) or a shorter soluble human ACE2 sequence (596 amino acids corresponding to residues 19-614 of SEQ ID NO: 1) or a Modified ACE2 sequence as defined above is PCR-amplified using the published human ACE2 sequence. The extracellular domain of human ACE2 as disclosed in FIG. 1 (SEQ ID NO:1) is fused to the N-terminus of Fc region of the human IgG1 or the human IgG3 (Uniprot no. P01860). The gene sequences for ACE2 as shown in FIG. 3 were optimized both in the codon usage and mRNA accumulation for expression in N. benthamiana, and then were synthesized (GENEWIZ, South Plainfield, N.J.). The optimized nucleotide sequence of ACE2 is provide in FIG. 1A. A point mutation was incorporated into the synthesized ACE2 sequence, producing an Arg to Lys amino acid change at position 273 to abolish its enzymatic activity [Guy 2005].

The ACE2 sequences or Modified ACE2 sequences were cloned into the pTRAk plant expression binary vector (Maclean et al. 2007) downstream of the signal peptide of the murine mAb24 heavy-chain, and upstream and in-frame with the Fc sequences from human IgG Fc sequences (hinge, CH2 and CH3) from human IgG1, IgG4, IgA1 or IgA2. The complete amino acid sequences of the in-frame Fc fusions with the Fc regions of IgG1, IgA1, IgA2, and IgG4 soluble huACE2 (aa 19-614 of SEQ ID NO: 1) and Modified ACE2 (aa 19-614 of SEQ ID NO: 1 with H34S aa substitution) are: huACE2-Fc(IgG1) (SEQ ID NO: 4) shown in FIG. 2A, Modified ACE2(H34S)-Fc(IgG1) (SEQ ID NO: 5) shown in FIG. 2B, huACE2-Fc(IgA1) (SEQ ID NO: 6) shown in FIG. 3A, huACE2-Fc(IgA2) (SEQ ID NO: 7) shown in FIG. 3B, and Modified huACE2(H34S)-Fc(IgG4) (SEQ ID NO: 16) shown in FIG. 13B.

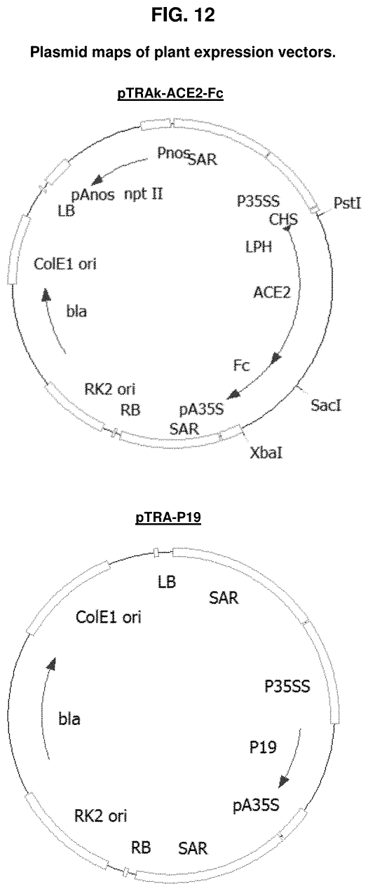

The corresponding DNA sequence is inserted in pTRAk as shown in FIG. 12 in the region denoted by ACE2 and Fc in the open reading frame (ORF). The IgA constructs are truncated to remove the 18-amino acid C-terminal IgA tail-piece, a sequence that enables dimeric IgA formation but significantly reduces IgA expression in plants (Hadlington et al. 2003) and is not required for binding Fc alpha receptors (Brunke et al. 2013). All constructs that included a C-terminal KDEL peptide for endoplasmic reticulum (ER) retention, result in high mannose N-glycans. Alternatively, without KDEL, the fusion protein is targeted to the plant cell secretory pathway via a signal peptide from a mouse antibody heavy chain. See FIG. 12, Plasmid maps for pTRAk-ACE2-Fc and pTRA-P19. The plasmid designated P1449 comprises the nucleotide sequence encoding the open reading frame of huACE2 (aa 19-614 of SEQ ID NO: 1) (273K)-human Fc1.

The resulting plasmids are transformed into A. tumefaciens GV3101::pMP90RK (Maclean et al. 2007) and the resulting A. tumefaciens strains are vacuum infiltrated into N. benthamiana for transient expression of the functional ACE2-Fc fusions. For high levels of expression, an Agrobacterium strain carrying a vector encoding the p19 protein of the tomato bushy stunt virus (Voinnet et al. 2003) to suppress post-transcriptional gene silencing is co-infiltrated. The Agrobacterium cell suspensions are combined and diluted to appropriate concentrations in infiltration buffer. Whole N. benthamiana plants (3-6 plants per pot), inverted and submerged into the bacterial suspension, are subjected to two sequences of vacuum (to 20 in. Hg for 10 sec) followed by slow vacuum release (˜2 kPa/second) to draw the bacterial suspension into the spongy leaf interstitial space. To produce proteins with complex glycosylation and reduced or absent xylose and fucose, the N. benthamiana plants will be DXT/FT. Following infiltration, plants are grown for up to 8 days in a greenhouse.

### Example 2: Extraction and Purification ACE2-Fc (IgG1) Fusion Proteins Produced in N. benthamiana

Briefly, N. benthamiana extracts are obtained by homogenizing the leaves with an aqueous buffer in a blender, which results in a mixture of the ACE2-Fc fusion protein and plant material. This mixture is clarified by centrifugation or other appropriate means such as filtration, which may be followed by micro filtration or ultrafiltration and or sterile filtration, followed by ACE2-Fc captured on columns of the appropriate affinity chromatography medium. IgG1 Fc fusions are purified using Protein A-Sepharose and IgA Fc fusions are purified using for example CaptureSelect™ Human IgA Affinity Matrix (Life Technologies) (Reinhart, Weik, and Kunert 2012). Other affinity chromatography resins, such as CaptureSelect IgA Affinity Matrix (Life Technologies) may be used for ACE2 IgA-Fc. fusions The ACE2-Fc fusions are eluted at low pH, neutralized, and dialyzed into PBS. Purity of 90-95% at >50% overall yield may be achieved. These affinity matrices work well with Fc-fusions and both have low affinity for plant proteins. If needed, an additional purification step, such as cation exchange chromatography, can be used.

In greater detail, upstream processing consists of grinding and pressing biomass, with an appropriate buffer (such as Tris, digested protein poly amines, ethylenediamine, PBS, pH 7.2-9.5) that maintain the stability and recovery of the ACE2-Fc in order to segregate solids from the product-containing Raw Juice. The Raw Juice may be treated with acid to pH 4.0-5.0 followed by base treatment to pH 7.2-8.5 or polyethyleneimine (PEI) at 0.025-0.1% (w/v) to agglomerate additional solids followed by centrifugation at 10K RPM for at least 15 min to remove solids and produce a clarified, product-containing liquid (centrate). The centrate is loaded onto Protein A, or other appropriate, affinity chromatography matrix.

The column is washed with 10-30 column volumes (CV) wash buffer containing PBS. Elution is carried out with 0.1 M glycine (acetic acid or citrate may also be used), 0.075-0.3 M NaCl, pH 2.0-3.0 and neutralized with 1 M HEPES, pH 8.0 or 1 M Tris, pH 8.5 (eluate). The eluate may be further purified via ion exchange chromatography and eluted via a salt or pH gradient. The polished eluate is buffer exchanged into the final formulation buffer and treated to remove endotoxin through a ToxinEraser (GenScript) column. Other excipients may be added to the final formulation to enhance stability and/or potency. The buffer exchanged eluate may be concentrated to the desired protein concentration and filtered through a 0.1-0.2 micron PES membrane prior to storage at or below −65° C.

Alternatively, the Protein A column is washed with 5-10 CV wash buffer containing 1% detergent (4 parts TX:114 to 1 part TX:100) in PBS. A second wash consist of 5-10 CV of 0.2 mg/ml Polymixin B in PBS. Lastly, 20 CV of PBS is used to wash away residual Polymixin B and/or detergent from the column prior to elution. Elution is carried out with 0.05-0.1 M glycine, 0.075-0.15 M NaCl, pH 2.0-3.0 and neutralized with 1 M HEPES, pH 8.0 or 1 M Tris, pH 8.5. The column may also be eluted using 0.75 M arginine (instead of glycine), 3.6 M MgCl₂ in 0.2 M acetate, pH 6.6, or combination thereof. The eluate is buffer exchanged into PBS via dialysis or diafiltration using 3.5-100 kDa cut-off regenerated cellulose, cellulose ester, or polyethersulfone (PES) membranes. Other excipients may be added to the final formulation to enhance stability and/or potency. The buffer exchanged eluate may be concentrated to the desired protein concentration and filtered through a 0.1-0.2 micron PES membrane prior to storage at or below −65° C.

### Example 3: Characterization of ACE2-Fc Fusion Proteins In Vitro

The structural integrity of the ACE2-Fc proteins is determined by reducing and non-reducing SDS-PAGE (Bio-Rad) and immunoblotting with Fc-specific antibodies (Southern Biotechnology) and ACE2-specific antibodies (R & D Systems). Depending upon the labeling method used for particular ACE2- and Fc-specific antibodies, different substrates are used to colorimetrically visualize binding. For example, alkaline phosphatase (AP) linked probes can be developed colorimetrically using BCIP/NBT substrate. Once catalyzed by AP, the product of BCIP (5-Bromo-4-chloro-3-indolyl phosphate) further reacts with NBT (nitro blue tetrazolium) to produce an insoluble precipitate that is dark blue to purple in color. BCIP/NBT substrate is supplied by numerous vendors such as Sigma Aldrich, Thermo-Fisher, and Promega.

Peroxidase (e.g., Horse Radish Peroxidase) linked probes can be developed colorimetrically using AEC substrate. AEC (3-Amino-9-ethylcarbazole) is a chromogenic substrate for horseradish peroxidase (HRP), a common antibody label in immunochemical applications. AEC reacts with hydrogen peroxide and HRP to yield a red-colored product that is soluble in alcohol. ACE substrate is supplied by numerous vendors such as those mentioned above and Vector Labs, Bic)Compare and Abcam.

Protein size, purity and assembly are determined by image analysis (Bio-Rad) of Coomassie stained (reduced and non-reduced) SDS-PAGE gels. The ACE2-Fc fusion proteins, derived from IgG1 IgA1, and IgA2 heavy chains, form homodimers under non-reducing conditions via disulfide bonds between hinge cysteines and have dimeric molecular weights.

Additional protein conformation characterization included analytical size exclusion chromatography (SEC) using a Shodex™ 8×300 mm column on a SpectraSYSTEM™ gradient HPLC (Thermo-Fisher). This column separates proteins between 500 and 1,000,000 Da. ACE2-Fc components are detected spectrophotometrically at 280 nm and quantified by measuring the area of individual peaks. Calibration of the column using protein molecular size standards allows accurately estimated sizes of ACE2-Fc fusion monomers, dimers, aggregates and fragments. The major peak will comprise approximately greater than 90% of the sample in fully dimeric form.

### Example 4: Extraction and Purification of ACE2-Fc Fusion Variants Produced in Nicotiana benthamiana

Four huACE2-Fc fusion variants were prepared using the plant (Nicotiana benthamiana) expression system. The variant fusion constructs prepared include ACE2 fused to IgG1 Fc with and without a flexible linker ((GGGGS)₃) between ACE2 and Fc, IgG3 Fc and IgG1 Fc containing two mutations (D270A/K322A) to knock out complement activating activity. All constructs, IgG3, contained the human IgG1 hinge. DNA sequences encoding the first 597 amino acids of the human ACE2 extracellular domain (aa 19-615 of SEQ ID NO: 1), codon optimized for tobacco expression, were cloned into the pTRAkc plant binary vector in frame with codon-optimized IgG1 Fc and IgG3 Fc region sequences, as shown in Table 3.

TABLE 3

| Construct # | First domain | Linker | Fc source |
|---|---|---|---|
| S2583 | huACE2 | None | IgG1 |
| S2585 | | (GGGGS)₂ | IgG1 |
| S2587 | | | IgG1 (D270A/K322A) |
| S2589 | | | IgG3 (IgG3 hinge replaced |
| | | | with IgG1 hinge) |

Recombinant A. tumefaciens strains carrying these expression vectors were used to transiently express ACE2-Fc in whole N. benthamiana plants following vacuum-assisted agroinfiltration. All samples were purified by Protein A affinity chromatography as described in Example 2 without subsequent polishing chromatography.

Protein concentrations were determined by 280 nm absorbance and estimated protein purity by densitometry using 4-20% TGX Stain-Free SDS-PAGE gels (Bio-Rad).

Alkaline phosphate (AP) linked probe was developed colorimetrically using BCIP/NBT substrate. Peroxidase (HRP) linked probe was developed colorimetrically using AEC substrate.

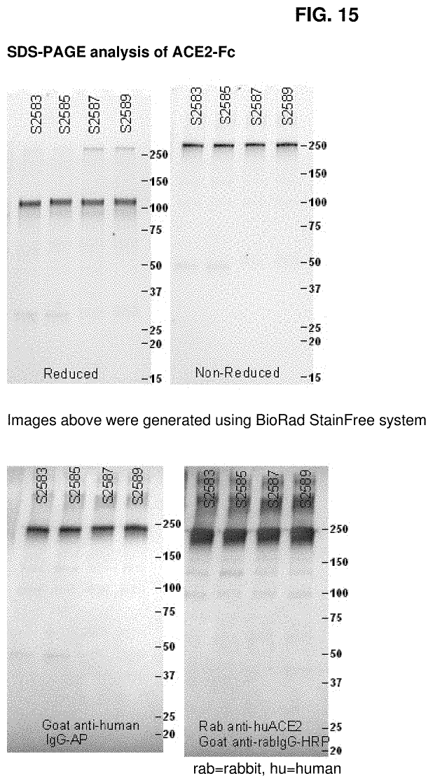

As shown in the gel images depicted in FIG. 15, the huACE2-Fc fusion variant constructs migrated at ˜250 kD under non-reducing condition which is above their predicted non-reduced size of ˜190 kD (based on their amino acid sequence) due to glycosylation of six N-linked glycosylation sites in the ACE2 sequence and one glycosylation site in the Fc. Signal at ˜250 kD for both anti-hu-IgG and anti-huACE2 Western blots indicated that the purified samples contains both ACE2 and Fc. Preliminary estimates of purified ACE2-Fc yield are ˜300 mg/kg fresh plant weight.

### Example 5: ELISA Assay of huACE2-Fc Fusion Variants Binding to SARS-COV-2 Spike Protein S1 domain

The ability of the soluble huACE2-Fc fusion variants to bind to the S1 domain of the SARS-COV-2 spike protein is determined in a functional ELISA. Briefly, SARS-COV-2 Spike protein S1 domain (Sino Biological, Cat #40591-V08H is coated on standard ELISA plates, at a concentration of 2 μg/mL in 1×PBS, 50 μL/well, and incubated for 60 min at 37° C., then blocked with 5% NFDM in 1×PBS, 15 min at 37° C. Samples containing the huACE2-Fc fusion constructs #2583, #2585, and #2589 prepared in Example 4 (see Table 3), or rhACE2 protein, were presented as a 3× serial dilution using blocking buffer, 50 L/well, and incubated for 60 min at 37° C. The wells are washed with blocking buffer, and bound huACE2-Fc fusion is detected using goat anti-human IgG HRP (Southern Biotech 1:2000 dilution), in a volume of 50 μl/well and incubated for 60 min at 37° C. OPD (o-Phenylenediamine dihydrochloride) substrate is added and absorbance at 490 nm is read on a Synergy™ HT Multi-Detection Microplate Reader (BioTek Instruments). The data is plotted and fitted to a 4-parameter logistic model (GraphPad, San Diego, Calif.). Results of the ELISA are reported in Table 4 below.

TABLE 4

| | Strain | | | | |
|---|---|---|---|---|---|
| | S2589 | S2585 | S2583 | | |
| | Protein | | | | |
| | ACE2-GS2- | ACE2-GS2- | ACE2- | | Neg |
| | hFc3 | hFc1 | hFc1 | rhACE2 | control |
| ELISA | 0.51 | 0.52 | 0.48 | 0.83 | >10 |

### Example 6: Construction of Additional huACE2-Fc Fusion Variants

Removal of Enzymatic Activity from ACE2

The single amino acid change at position 273 of SEQ ID NO: 1 from R to Q or K eliminates the enzymatic activity of huACE2 and huACE2-Fc fusions. This single amino acid change has no effect on folding ACE2 in the region of the S1 binding site. The amino acid changes can be made to the corresponding nucleic acid codons via overlap extension PCR mutagenesis, by using a site-directed mutagenesis kit (Q5® Kit, New England Biolabs), or by commercially available de novo synthesis of the corresponding nucleic acid sequence by means well known in the industry.

ACE2 enzymatic activity can be measured using a fluorogenic assay with the synthetic ACE2 substrate, Mca-APK(Dnp) (Guy et al. 2005). The assay is monitored continuously by measuring the increase in fluorescence (excitation=340 nm, emission=430 nm) upon substrate hydrolysis using a Wallac Victor² fluorescence plate reader (Turku, Finland). Initial velocities are determined from the linear rate of fluorescence increase over the 0-60 min time course. The reaction product is quantified by using standard solutions of Mca.

Production of ACE2 Amino Acid Variants.

ACE2 amino acid variants are made by making one or more amino acid substitutions in the ACE2 gene sequence. This is done by changing the DNA codon sequence using methods such as overlap extension PCR mutagenesis (see e.g., “A rapid and efficient method for site-directed mutagenesis using one-step overlap extension PCR,” Andreas Urban, Susanne Neukirchen, Karl-Erich Jaeger Nucleic Acids Research, Vol, 25, issue 11, 1 Jun. 1997, Pages 2227-2228, https://doi.org/10.1093/nar/25.11.2227) which is incorporated herein by reference. Such an overlap extension PCR can be carried out using a site-directed mutagenesis kit as described hereinabove, or by gene synthesis.

Examples of codon modifications to produce amino acid sequence variants (substitutions) of huACE2 are shown below in Table 5. The amino acid sequence numbering of these amino acid substitutions is based on the numbering of the 805 aa huACE2 amino acid sequence of SEQ ID NO: 1. Primers for producing the specific codon changes shown below by the above-indicated methods are readily designed by those skilled in the art.

TABLE 5

| Amino Acid Modification | Example of Codon |
|---|---|
| (relative to SEQ ID NO: 1) | Change |
| D30E | GAC > GAG |
| D30S | GAC > TCC |
| K31Q | AAG > CAG |
| K31E | AAG > GAG |
| H34S | CAC > TCC |
| H34V | CAC > GTG |
| D38E | GAC > GAG |
| F40S | TTC > TCC |
| Q42A | CAG > GCC |
| Q81K | CAG > AAG |
| M82N | ATG > AAC |
| M82K | ATG > AAG |
| M82T | ATG > ACC |
| E329N | GAG > AAC |
| G354H | GGA > CAC |
| G354K | GGA > AAG |
| S19P | TCC > CCG |
| N103S | AAC > TCG |
| K341R | AAG > CGC |
| I468V | ATC > GTC |
| N546S | AAC > AGC |
| S547P | TCC > CCG |
| T548A | ACC > GCG |

Specifically, oligonucleotide primers for producing the above enumerated specific codon changes by the above-indicated methods are shown below in Tables 6 and 6a, in which the codon that is altered is underlined within the sequence of the primer.

TABLE 6

| Amino | | | SEQ |
|---|---|---|---|
| Acid | Codon | Primers for site directed mutagenesis or | ID |
| Change | Change | overlap extension PCR | NO: |
| D30E | GAC > GAG | 5′-CAGGCTAAGACCTTCCTCGAGAAGTTCAACCACGAGGCC | 50 |
| | | 5′-GGCCTCGTGGTTGAACTTCTCGAGGAAGGTCTTAGCCTG | 51 |
| D30S | GAC > TCC | 5′-CAGGCTAAGACCTTCCTCTCCAAGTTCAACCACGAGGCC | 52 |
| | | 5′-GGCCTCGTGGTTGAACTTGGAGAGGAAGGTCTTAGCCTG | 53 |
| K31Q | AAG > CAG | 5′-GCTAAGACCTTCCTCGACCAGTTCAACCACGAGGCCGAG | 54 |
| | | 5′-CTCGGCCTCGTGGTTGAACTGGTCGAGGAAGGTCTTAGC | 55 |
| K31E | AAG > GAG | 5′-GCTAAGACCTTCCTCGACGAGTTCAACCACGAGGCCGAG | 56 |
| | | 5′-CTCGGCCTCGTGGTTGAACTCGTCGAGGAAGGTCTTAGC | 57 |
| H34S | CAC > TCC | 5′-TTCCTCGACAAGTTCAACTCCGAGGCCGAGGACCTCTTC | 58 |
| | | 5′-GAAGAGGTCCTCGGCCTCGGAGTTGAACTTGTCGAGGAA | 59 |
| H34V | CAC > GTG | 5′-TTCCTCGACAAGTTCAACGTGGAGGCCGAGGACCTCTTC | 60 |
| | | 5′-GAAGAGGTCCTCGGCCTCCACGTTGAACTTGTCGAGGAA | 61 |
| D38E | GAC > GAG | 5′-TTCAACCACGAGGCCGAGGAGCTCTTCTACCAGTCCTCC | 62 |
| | | 5′-GGAGGACTGGTAGAAGAGCTCCTCGGCCTCGTGGTTGAA | 63 |
| Q42A | CAG > GCC | 5′-GCCGAGGACCTCTTCTACGCCTCCTCCCTCGCCTCCTGG | 64 |
| | | 5′-CCAGGAGGCGAGGGAGGAGGCGTAGAAGAGGTCCTCGGC | 65 |
| M82N | ATG > AAC | 5′-GCAGTCCACCCTCGCCCAGAACTACCCACTCCAGGAGAT | 66 |
| | | 5′-ATCTCCTGGAGTGGGTAGTTCTGGGCGAGGGTGGACTGC | 67 |
| M82K | ATG > AAG | 5′-CAGTCCACCCTCGCCCAGAAGTACCCACTCCAGGAGATC | 68 |
| | | 5′-GATCTCCTGGAGTGGGTACTTCTGGGCGAGGGTGGACTG | 69 |
| G354H | GGA > CAC | 5′-GCCTGGGACCTCGGAAAGCACGACTTCAGGATCCTCATG | 70 |
| | | 5′-CATGAGGATCCTGAAGTCGTGCTTTCCGAGGTCCCAGGC | 71 |
| G354K | GGA > AAG | 5′-GCCTGGGACCTCGGAAAGAAGGACTTCAGGATCCTCATG | 72 |
| | | 5′-CATGAGGATCCTGAAGTCCTTCTTTCCGAGGTCCCAGGC | 73 |
| D38E + | GAC > GAG | 5′- | 74 |
| F40S | TTC > TCC | CAACCACGAGGCCGAGGAGCTCTCCTACCAGTCCTCCCTCGC | |
| | | 5′- | 75 |
| | | GCGAGGGAGGACTGGTAGGAGAGCTCCTCGGCCTCGTGGTTG | |
| Q81K + | CAG > AAG | 5′-GAGCAGTCCACCCTCGCCAAGAACTACCCACTCCAGGAGAT | 76 |
| M82N | ATG > AAC | 5′- | 77 |
| | | ATCTCCTGGAGTGGGTAGTTCTTGGCGAGGGTGGACTGCTC | |
| M82T | ATG > ACC | 5′-CAGTCCACCCTCGCCCAGACCTACCCACTCCAGGAGATC | 78 |
| | | 5′-GATCTCCTGGAGTGGGTAGGTCTGGGCGAGGGTGGACTG | 79 |
| E329N | GAG > AAC | 5′-ATGACCCAAGGATTCTGGAACAACTCCATGCTCACCGAT | 80 |
| | | 5′-ATCGGTGAGCATGGAGTTGTTCCAGAATCCTTGGGTCAT | 81 |
| S19P | TCC > CCG | 5′-CTGCAGGTGTTCACTCCCCGACCATCGAGGAACAGGCTA | 82 |
| | | 5′-TAGCCTGTTCCTCGATGGTCGGGGAGTGAACACCTGCAG | 83 |
| N103S | AAC > TCG | 5′-CTCCAGGCCCTCCAGCAGTCGGGATCCTCCGTGCTCTCC | 84 |
| | | 5′-GGAGAGCACGGAGGATCCCGACTGCTGGAGGGCCTGGAG | 85 |
| K341R | AAG > CGC | 5′-ATCCCGGCAACGTGCAGCGCGCCGTCTGCCACCCCACCG | 86 |
| | | 5′-CGGTGGGGTGGCAGACGGCGCGCTGCACGTTGCCGGGAT | 87 |
| I468V | ATC > GTC | 5′-TGGTGTTCAAGGGCGAGGTCCCCAAGGACCAGTGGATGA | 88 |
| | | 5′-TCATCCACTGGTCCTTGGGGACCTCGCCCTTGAACACCA | 89 |
| N546S | AAC > AGC | 5′-ACAAGTGCGACATCTCCAGCTCCACCGAGGCCGGCCAGA | 90 |
| | | 5′-TCTGGCCGGCCTCGGTGGAGCTGGAGATGTCGCACTTGT | 91 |
| S547P | TCC > CCG | 5′-AGTGCGACATCTCCAACCCGACCGAGGCCGGCCAGAAAC | 92 |
| | | 5′-GTTTCTGGCCGGCCTCGGTCGGGTTGGAGATGTCGCACT | 93 |
| T548A | ACC > GCG | 5′-GCGACATCTCCAACTCCGCGGAGGCCGGCCAGAAACTCT | 94 |
| | | 5′-AGAGTTTCTGGCCGGCCTCCGCGGAGTTGGAGATGTCGC | 95 |
| S19P | TCC > CCG | 5′-ATCCCGGCAACGTGCAGCGCGCCGTCTGCCACCCCACCG | 96 |
| | | 5′-ATCCCGGCAACGTGCAGCGCGCCGTCTGCCACCCCACCG | 97 |
| H34V | CAC > GTG | 5′-TTCCTCGACAAGTTCAACGTGGAGGCCGAGGACCTCTTC | 98 |
| | | 5′-GAAGAGGTCCTCGGCCTCCACGTTGAACTTGTCGAGGAA | 99 |

As presented in Table 6, the number of the amino acid residue in the column designated “Amino Acid Change” refers to the amino acid residue in the huACE2 sequence of SEQ ID NO: 1 (as depicted in FIG. 1). In terms of the huACE2-Fc Modified and ACE2-Fc proteins that include only the soluble huACE2 protein of amino acids 19-614 of SEQ ID NO: 1 linked to a Fc region at the carboxy terminus, the amino acid D30 of SEQ ID NO: 1 is at position 12. Thus, the amino acid D30 corresponds to amino acid D12 of the various ACE2-Fc fusion proteins of SEQ ID NO: 4, 5, 6, 7, 11, 13, and 15 (shown in FIGS. 3, 4, 8, 10, and 13A).

Similarly, in those Fc-huACE2 fusion proteins where the Fc region segment precedes the ACE2 segment in the enumeration of the overall sequence, the amino acid D30 of the ACE2 sequence of SEQ ID NO: 1 corresponds to amino acid residue number based on the particular length of IgG-Fc region that is linked to the N-terminus of the huACE2 sequence. For example, D30 corresponds to D245 of the complete (IgG1) Fc-ACE2 fusion amino acid sequence of SEQ ID NO: 8 (shown in FIG. 5).

Production of Additional Specific Variant sequences of ACE2 by PCR Mutagenesis

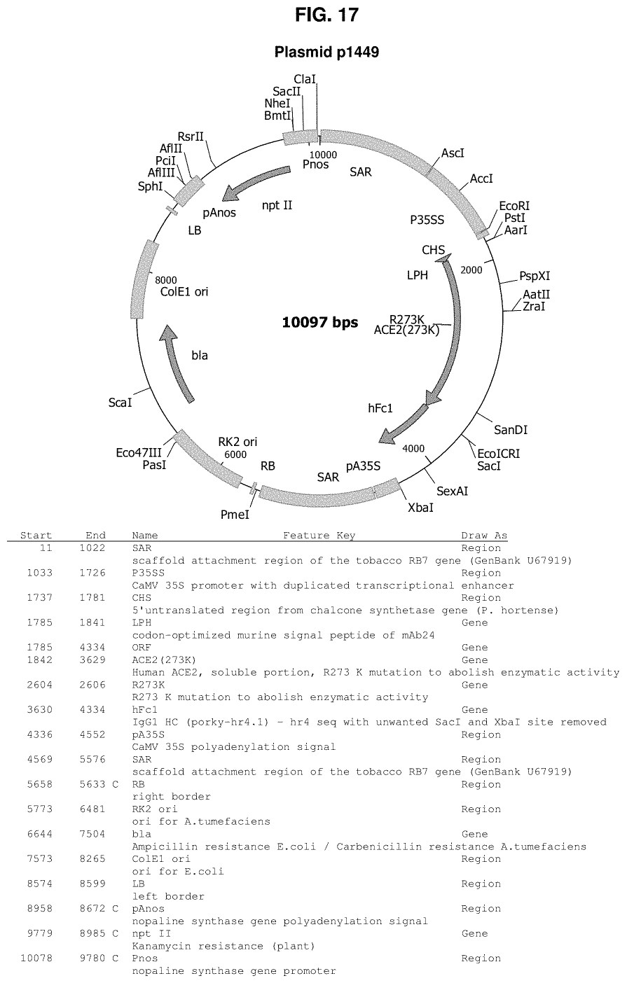

As shown in Table 6A, the expression vectors for production of ACE2 with the sequence modifications listed in Table 6 are constructed to produce intermediate plasmids p1500 to p1506 using the designated PCR primer pairs for each of the desired amino acid modifications. The template plasmid p1449, a map of which is depicted in FIG. 17, is used in each construction. Table 6A also lists the primer extension sequence used for each of PCR mutagenesis.

TABLE 6A

| Plasmid | Construct | Primer # | Primer sequence |
|---|---|---|---|
| p1500 | pTRAk-c-lph- | O1311 | CTGCAGGTGTTCACTCCCCGACCATCGA |
| | ACE2(19P, 273K)-hFc1 | | GGAACAGGCTA (SEQ ID NO: 100) |
| p1500 | pTRAk-c-lph- | O1312 | TAGCCTGTTCCTCGATGGTCGGGGAGTG |
| | ACE2(19P, 273K)-hFc1 | | AACACCTGCAG (SEQ ID NO: 101) |
| p1501 | pTRAk-c-lph- | O1313 | CTCCAGGCCCTCCAGCAGTCGGGATCCT |
| | ACE2(103S, 273K)-hFc1 | | CCGTGCTCTCC (SEQ ID NO: 102) |
| p1501 | pTRAk-c-lph- | O1314 | GGAGAGCACGGAGGATCCCGACTGCTGG |
| | ACE2(103S, 273K)-hFc1 | | AGGGCCTGGAG (SEQ ID NO: 103) |
| p1502 | pTRAk-c-lph- | O1315 | ATCCCGGCAACGTGCAGCGCGCCGTCTG |
| | ACE2(341R, 273K)-hFc1 | | CCACCCCACCG (SEQ ID NO: 104) |
| p1502 | pTRAk-c-lph- | O1316 | CGGTGGGGTGGCAGACGGCGCGCTGCAC |
| | ACE2(341R, 273K)-hFc1 | | GTTGCCGGGAT (SEQ ID NO: 105) |
| p1503 | pTRAk-c-lph- | O1317 | TGGTGTTCAAGGGCGAGGTCCCCAAGGA |
| | ACE2(468V, 273K)-hFc1 | | CCAGTGGATGA (SEQ ID NO: 106) |
| p1503 | pTRAk-c-lph- | O1318 | TCATCCACTGGTCCTTGGGGACCTCGCC |
| | ACE2(468V, 273K)-hFc1 | | CTTGAACACCA (SEQ ID NO: 107) |
| p1504 | pTRAk-c-lph- | O1319 | ACAAGTGCGACATCTCCAGCTCCACCGA |
| | ACE2(546S, 273K)-hFc1 | | GGCCGGCCAGA (SEQ ID NO: 108) |
| p1504 | pTRAk-c-lph- | O1320 | TCTGGCCGGCCTCGGTGGAGCTGGAGAT |
| | ACE2(546S, 273K)-hFc1 | | GTCGCACTTGT (SEQ ID NO: 109) |
| p1505 | pTRAk-c-lph- | O1321 | AGTGCGACATCTCCAACCCGACCGAGGC |
| | ACE2(547P, 273K)-hFc1 | | CGGCCAGAAAC (SEQ ID NO: 110) |
| p1505 | pTRAk-c-lph- | O1322 | GTTTCTGGCCGGCCTCGGTCGGGTTGGA |
| | ACE2(547P, 273K)-hFc1 | | GATGTCGCACT (SEQ ID NO: 111) |
| p1506 | pTRAk-c-lph- | O1323 | GCGACATCTCCAACTCCGCGGAGGCCGG |
| | ACE2(548A, 273K)-hFc1 | | CCAGAAACTCT (SEQ ID NO: 112) |
| p1506 | pTRAk-c-lph- | O1324 | AGAGTTTCTGGCCGGCCTCCGCGGAGTT |
| | ACE2(548A, 273K)-hFc1 | | GGAGATGTCGC (SEQ ID NO: 113) |
| p1500 | pTRAk-c-lph- | 01325 | GAAACTGCAGGTGTTCACTCCCCGACCA |
| | ACE2(19P, 273K)-hFc1 | | TCGAGGAACAGGCTA (SEQ ID NO: |
| | | | 114) |
| p1461 | pTRAk-c-lph- | 01257 | TTCCTCGACAAGTTCAACTCCGAGGCCG |
| | ACE2(34S, 273K)-hFc1 | | AGGACCTCTTC (SEQ ID NO: 115) |
| p1461 | pTRAk-c-lph- | 01258 | GAAGAGGTCCTCGGCCTCGGAGTTGAAC |
| | ACE2(34S, 273K)-hFc1 | | TTGTCGAGGAA (SEQ ID NO: 116) |
| p1468 | pTRAk-c-lph-ACE2(19- | O1272 | TTCCTCGACAAGTTCAACGTGGAGGCCG |
| | 614, 34V, 273K)- hFc1 | | AGGACCTCTTC (SEQ ID NO: 117) |
| p1468 | pTRAk-c-lph-ACE2(19- | O1273 | GAAGAGGTCCTCGGCCTCCACGTTGAAC |
| | 614, 34V, 273K)-hFc1 | | TTGTCGAGGAA (SEQ ID NO: 118) |
| p1473 | pTRAk-c-lph-ACE2(19- | 01280 | TCCTCGGAGATATGTGGGGAAgGTTCTG |
| | 614)-hFc1 | | GACCAACCTCTACT (SEQ ID NO: |
| | | | 119) |
| P1473 | pTRAk-c-lph-ACE2-(19- | 01281 | AGTAGAGGTTGGTCCAGAACcTTCCCCA |
| | 614)hFc1 | | CATATCTCCGAGGA (SEQ ID NO: |
| | | | 120) |

Table 6B lists the particular amino acid modifications of the ACE2 sequence resulting from the PCR mutagenesis, the expression vector construct containing the modified amino acid sequence of ACE2 and human Fc sequence, the type of sequence change produced by the alteration of the ACE2 sequence and the utility obtained by producing the ACE2 sequence change and preferred combination of the sequence change with additional alterations in the ACE2 amino acid sequence.

TABLE 6B

| Amino Acid | Codon | Type of Sequence | |
|---|---|---|---|
| Modification | Change | Change | Utalization |
| S19P | TCC > CCG | human variant | improved binding, combine with |
| | | | H34S or other ACE2 altered amino |
| | | | acid residue |
| N103S | AAC > TCG | removes | improved binding, combine with |
| | | glycosylation | H34S or other ACE2 altered amino |
| | | | acid residue improved binding, |
| | | | combine with H34S |
| K341R | AAG > CGC | human variant | improved binding, combine with |
| | | | H34S or other ACE2 altered amino |
| | | | acid residue |
| I468V | ATC > GTC | human variant | improved binding, combine with |
| | | | H34S or other ACE2 altered amino |
| | | | acid residue |
| N546S | AAC > AGC | human variant no | improved binding, combine with |
| | | glycosylation | H34S or other ACE2 altered amino |
| | | at 546 | acid residue |
| S547P | TCC > CCG | removes | improved binding, combine with |
| | | glycosylation | H34S or other ACE2 altered amino |
| | | at 546 | acid residue |
| H34V | CAC > GTG | Changes residue | improved binding, combine with other |
| | | 34 from H to V | ACE2 altered amino acid residue |

Each ACE2 variant construct is produced in a three-step process in which each mutagenesis primer is utilized with template parent plasmids to form PCR sequences A and B (e.g., p1500A and p1500B). The two PCR sequences A and B produced on the template are combined and annealed to produce the final PCR sequence C (e.g., p1500C) with the complete sequence encoding the desired amino acid alteration in the sequence of ACE2.

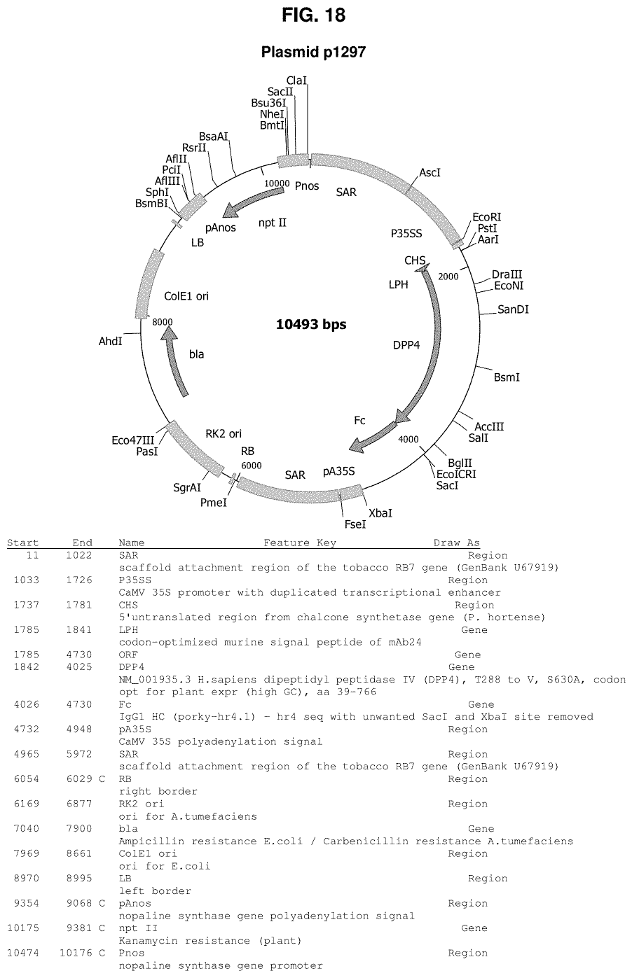

After annealing and then amplification with end compatible primers, PCR sequence C is cloned into plasmid p1297 (shown in FIG. 18) to form the expression vector. Two specific primers are used in the construction of each of the PCR sequences A and B and the final plasmids: Primer 0452 has the sequence TGGAGTGGAGCTGGATCTTC (SEQ ID NO: 121); Primer 01240 has the sequence GAAAGAGCTCGCATAAGGGGACCAGTCGGT (SEQ ID NO: 122). The steps of the three step processes used are indicated in Table 6C below. In each of the following three step processes the specific primer sequences utilized in addition to primer 0452 and 01240 are listed in Table 6A.

TABLE 6C
Three Step Process of PCR mutagenesis

| | | |
|---|---|---|
| PCR1461 | | |
| PCR1461-A | PCR1461-B | PCR1461-C |
| Template p1449 | Template p1449 | Template PCRs A + B |
| Primers O452, O1258 | Primers O1257, O1240 | Primers O452, O1240 |
| Anneal @ 52 C. | Anneal @ 62 C. | 5 cycles at annealing temp 72 C., then |
| Product 122 bp | Product 1770 bp | anneal at 54 C. for 30 cycles |
| | | Product 1853 bp |
| PCR1473 | | |
| PCR1473-A | PCR1473-B | PCR1473-C |
| Template p1449 | Template p1449 | Template PCRs A + B |
| Primers O452, O1281 | Primers O1280, O1240 | Primers O452, O1240 |
| Anneal @ 56 C. | Anneal @ 62 C. | 5 cycles at annealing temp 72 C., then |
| Product 840 bp | Product 1055 bp | anneal at 56 C. for 30 cycles |
| | | Product 1853 bp |
| PCR1500 | | |
| PCR1500-A | PCR1500-B | PCR1500-C |
| Template p1449 | Template p1449 | Template PCRs A + B |
| Primers O452, O1312 | Primers O1311, O1240 | Primers O452, O1240 |
| Anneal @ 52 C. | Anneal @ 62 C. | 5 cycles at annealing temp 72 C., |
| Product 122 bp | Product 1770 bp | then anneal at 52 C. for 30 cycles |
| | | Product 1853 bp |
| PCR1501 | | |
| PCR1501-A | PCR1501-B | PCR1501-C |
| Template p1449 | Template p1449 | Template PCRs A + B |
| Primers O452, 01314 | Primers 01313, O1240 | Primers O452, O1240 |
| Anneal @ 55 C. | Anneal @ 62 C. | 5 cycles at annealing temp 72 C., |
| Product 329 bp | Product 1563 bp | then anneal at 55 C. for 30 cycles |
| | | Product 1853 bp |
| PCR1502 | | |
| PCR1502-A | PCR1502-B | PCR1502-C |
| Template p1449 | Template p1449 | Template PCRs A + B |
| Primers O452, 01316 | Primers 01315, O1240 | Primers O452, O1240 |
| Anneal @ 56 C. | Anneal @ 62 C. | 5 cycles at annealing temp 72 C., |
| Product 1044 bp | Product 848 bp | then anneal at 56 C. for 30 cycles |
| | | Product 1853 bp |
| PCR1503 | | |
| PCR1503-A | PCR1503-B | PCR1503-C |
| Template p1449 | Template p1449 | Template PCRs A + B |
| Primers O452, 01318 | Primers 01317, O1240 | Primers O452, O1240 |
| Anneal @ 56 C. | Anneal @ 61 C. | 5 cycles at annealing temp 72 C., |
| Product 1425 bp | Product 467 bp | then anneal at 56 C. for 30 cycles |
| | | Product 1853 bp |
| PCR1504 | | |
| PCR1504-A | PCR1504-B | PCR1504-C |
| Template p1449 | Template p1449 | Template PCRs A + B |
| Primers O452, 01320 | Primers 01319, O1240 | Primers O452, O1240 |
| Anneal @ 57 C. | Anneal @ 60 C. | 5 cycles at annealing temp 72 C., |
| Product 1659 bp | Product 233 bp | then anneal at 57 C. for 30 cycles |
| | | Product 1853 bp |
| PCR1505 | | |
| PCR1505-A | PCR1505-B | PCR1505-C |
| Template p1449 | Template p1449 | Template PCRs A + B |
| Primers O452, 01322 | Primers 01321, O1240 | Primers O452, O1240 |
| Anneal @ 56 C. | Anneal @ 60 C. | 5 cycles at annealing temp 72 C., |
| Product 1662 bp | Product 230 bp | then anneal at 56 C. for 30 cycles |
| | | Product 1853 bp |
| PCR1506 | | |
| PCR1506-A | PCR1506-B | PCR1506-C |
| Template p1449 | Template p1449 | Template PCRs A + B |
| Primers O452, 01324 | Primers 01323, O1240 | Primers O452, O1240 |
| Anneal @ 56 C. | Anneal @ 60 C. | 5 cycles at annealing temp 72 C., |
| Product 1665 bp | Product 227 bp | then anneal at 56 C. for 30 cycles |
| | | Product 1853 bp |

A summary of the formation of the various mutagenesis expression vectors is provided in sections below.

Clone p1461 pTRAk-c-lph-ACE2(34S,273K)-hFc1

Vector: p1297, cut with PstI and SacI, CiP treat—keep 8293 bp, discard 2200 bp. Insert: Cut PCR1461-C with PstI and SacI. Keep 1804 bp, discard 44.5 bp.

Clone p1473 pTRAk-c-lph-ACE2-hFc1

Vector: p1297, cut with PstI and SacI, CiP treat—keep 8293 bp, discard 2200 bp. Insert: Cut PCR1473-C with PstI and SacI. Keep 1804 bp, discard 44.5 bp.

Clone p1500 pTRAk-c-lph-ACE2(19-614,19P,273K)-hFc1

Vector: p1297, cut with PstI and SacI, CiP treat—keep 8293 bp, discard 2200 bp. Insert: Cut PCR1500-C with PstI and SacI. Keep 1804 bp, discard 44.5 bp

Clone p1501 pTRAk-c-lph-ACE2(19-614,103S,273K)-h Fc1

Vector: p1297, cut with PstI and SacI, CiP treat—keep 8293 bp, discard 2200 bp. Insert: Cut PCR1501-C with PstI and SacI. Keep 1804 bp, discard 44.5 bp

Clone p1502 pTRAk-c-lph-ACE2(19-614,341R,273K)-hFc1

Vector: p1297, cut with PstI and SacI, CiP treat—keep 8293 bp, discard 2200 bp. Insert: Cut PCR1502-C with PstI and SacI. Keep 1804 bp, discard 44.5 bp

Clone p1503 pTRAk-c-lph-ACE2(19-614,468V,273K)-h Fc1

Vector: p1297, cut with PstI and SacI, CiP treat—keep 8293 bp, discard 2200 bp Insert: Cut PCR1503-C with PstI and SacI. Keep 1804 bp, discard 44.5 bp

Clone p1504 pTRAk-c-lph-ACE2(19-614,546S,273K)-h Fc1

Vector: p1297, cut with PstI and SacI, CiP treat—keep 8293 bp, discard 2200 bp. Insert: Cut PCR1504-C with PstI and SacI. Keep 1804 bp, discard 44.5 bp.

Clone p1505 pTRAk-c-lph-ACE2(19-614,547P,273K)-hFc1

Vector: p1297, cut with PstI and SacI, CiP treat—keep 8293 bp, discard 2200 bp. Insert: Cut PCR1505-C with PstI and SacI. Keep 1804 bp, discard 44.5 bp.

Clone p1506 pTRAk-c-lph-ACE2(19-614,548A,273K)-hFc1

Vector: p1297, cut with PstI and SacI, CiP treat—keep 8293 bp, discard 2200 bp. Insert: Cut PCR1506-C with PstI and SacI. Keep 1804 bp, discard 44.5 bp.

Clone p1500 pTRAk-c-lph-ACE2(19-614,19P,273K)-hFc1

Vector: p1297, cut with PstI and SacI, CiP treat—keep 8293 bp, discard 2200 bp. Insert: Cut PCR1500 with PstI and SacI. Keep 1804 bp, discard 9.5 bp.

Alternatively, PCR1500 can be done simply with the primer 01325 and O1240 using p1449 as a template. The primer contains the PstI site. This can then be cloned into p1297 using PstI and SacI. The alternative PCR1500 parameters are as follows: Template p1449; Primers 01325, O1240; Anneal: 62C; Product 1818 bp.

Examples A-J below describe further details of the preparation of specific variant ACE2-Fc or Fc-ACE2 fusion constructs, with the primers used to perform the site-directed mutagenesis or overlap extension PCR are those listed in Table 6A

A. p1468 pTRAk-c-lph-ACE2(19-614,34V,273K)-hFc1

In this example A, the ACE2 sequence is modified by either site directed mutagenesis or overlap extension PCR to change the codon CAC to GTG in the position encoding residue 34 in the ACE2 sequence 19-614,273K to change residue 34 from H to V as follows forming plasmid p1468 pTRAk-c-lph-ACE2(19-614,34V,273K)-hFc1:

Using plasmid p1449 (FIG. 17) as template two sequences PCR1468-A (122 base pairs) and PCR1468B (1770) base pairs are produced. PCR 1468-A is produced using primer pair 0452 and O1273 (Table 6A) and PCR 1468-B is produced using primer pair 01272 (Table 6A) and 01240. PCR1468-A and PCR-B are combined with primers O452 and O124 and cycled 5 times at annealing temperature of 72C and then annealed at 52C for 30 cycles producing PCR-1468-c, a1853 base pair sequence with the desired codon modification. To clone the desired sequence into an expression vector, plasmid p1297 (FIG. 18) is cut with restriction endonucleases Pst 1 and Sac 1, treated with Calf intestinal Phosphatase (CiP) to dephosphorylate the 5′ and 3′ ends of the sequences. The resulting 8293 bp sequence is retained and the 2200 bp fragment is discarded. PCR1468-C is cut with restriction endonucleases Pst I and Sac I, the 1894 bp sequence is retained and combined with the 8293 bp sequence under conditions allowing the sequences to join to produce p1468 pTRAk-c-lph-ACE2(19-614,34V,273K)-hFc1

B. p1511 pTRAk-c-lph-ACE2(19-614,546S)-hFc1

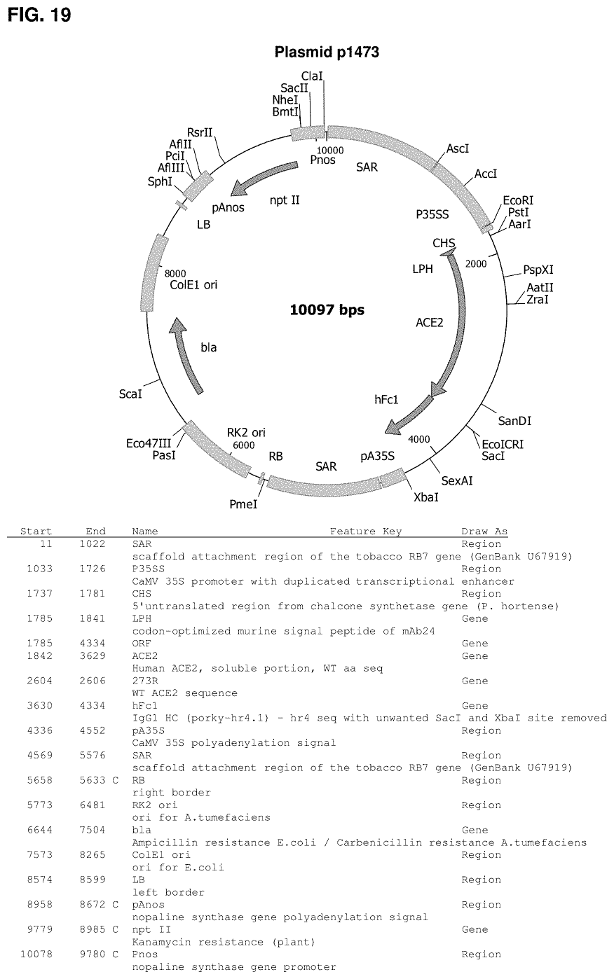

In this example B, the ACE2 sequence is modified by either site directed mutagenesis or overlap extension PCR to change the codon AAC>AGC in the position encoding residue 546 in the ACE2 sequence 19-614 from N to S as follows forming plasmid p1511. The plasmid template used in this example to form P1511 in a three-step process is plasmid p1473 (shown in FIG. 19).

Using plasmid p1473 (FIG. 19) as template two sequences PCR1511-A (1659 base pairs) and PCR1511-B (233 base pairs) are produced by annealing at 57C and 60C respectively. PCR 1511-A is produced using primer pair 0452 and 01320 (Table 6A). PCR 1511-B is produced using primer pair O1319 (Table 6A) and 01240. PCR1511-A and PCR-1511 B are combined with primers O452 and O1240 and cycled 5 times at annealing temperature of 72C and then annealed at 57C for 30 cycles producing PCR-1511-C, a 1853 base pair sequence with the desired codon modification. To clone the desired sequence into an expression vector, plasmid p1297 (FIG. 18) is cut with restriction endonucleases Pst1 and Sac1, treated with Calf intestinal phosphatase (CiP) to dephosphorylate the 5′ and 3′ ends of the sequences. The resulting 8293 bp sequence is retained and the 2200 bp fragment is discarded. PCR 1511-C is cut with restriction endonucleases Pst I and Sac I the 1804 bp sequence is retained and combined with the 8293 bp sequence under conditions allowing the sequences to join to produce p1511 pTRAk-c-lph-ACE2(19-614,546S)-hFc1

C. p1512 pTRAk-c-lph-ACE2(19-615,546S)-(GGGGS)₂-hFc1

In this example C, plasmid p1512 is derived from plasmid p1511 and has the linker (GGGGS)₂ interposed between the modified ACE2 sequence and the Fc sequence. Plasmid p1512 has the structure pTRAk-c-lph-ACE2(19-615,546S)-(GGGGS)₂-hFc1 and is produced as follows. Using plasmid p1511 as template, primers 0722 having the sequence CCTTCGCAAGACCCTTCCTC (SEQ ID NO: 123) and 01316 (see Table 6A) are annealed at 53C to produce a 1996 base pair sequence.

Plasmid p1473 (shown in FIG. 19) is cut with PstI and Sac I, treated with CiP. The resulting 8293 bp sequence is retained and the 1804 bp fragment is discarded. The 8293 bp sequence is combined with the 1996 bp sequence produced in the previous step, which includes the ACE2, 546S with the long linker sequence (GGGGS)₂ and is cut with Pst I and Sac I. The resulting 1834 bp sequence is retained and the 157 and 5 bp fragments are discarded. The retained sequences are treated under conditions allowing the retained fragments to join to form the clone p1512 having the structure pTRAk-c-lph-ACE2(19-615,5465)-(GGGGS)₂-hFc1.

D. p1513 pTRAk-c-lph-ACE2(19-615,5465,273K)-(GGGGS)₂-hFc1

In this example D, the 273K mutation is introduced to form p1513. Using previously produced plasmid p1504 as template, primers 0722 and 01326 (see Table 6A) are annealed at 53C to produce a 1996 base pair sequence. Previously produced Plasmid p1507, is cut with Pst I and Sac I, and treated with CiP. The resulting 8293 bp sequence is retained and the 1804 bp fragment is discarded. The 8293 bp sequence is combined with the 1996 bp sequence which includes the sequence ACE2, 273k, 546S with long linker sequence (GGGGS)₂, and cut with Pst I and Sac I. The resulting 1834 bp sequence is retained and the 157 and 5 bp fragments are discarded. The retained sequences are treated under conditions allowing the retained sequences to join to form the p1513 having the structure pTRAk-c-lph-ACE2(19-615,546S,273K)-(GGGGS)₂-hFc1.

E. p1514 pTRAk-c-lph-ACE2(19-615,34S,546S)-(GGGGS)₂-hFc1

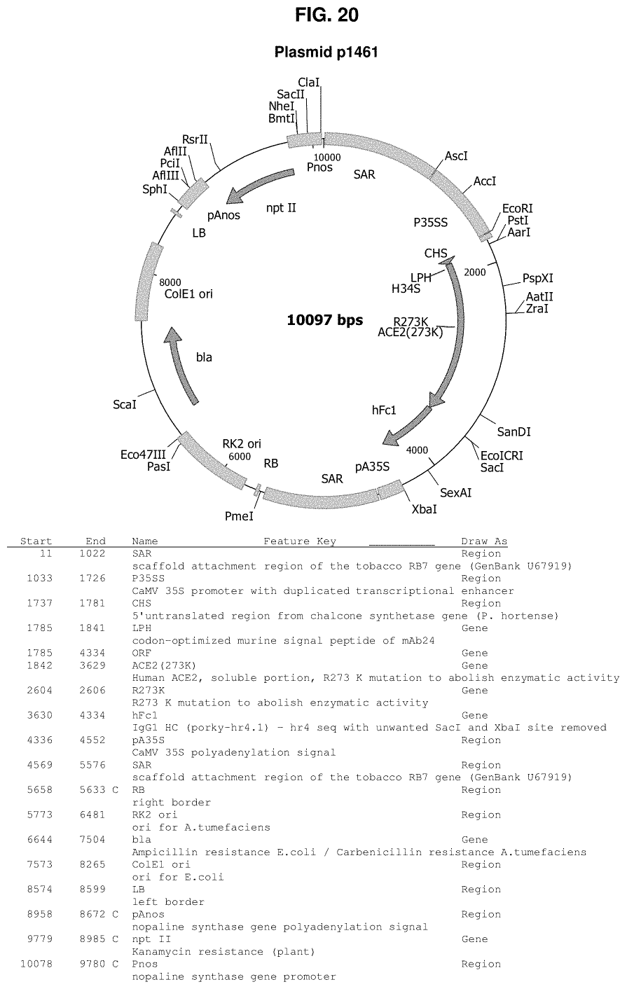

In this example E, the 34S mutation is introduced into a vector p1512 previously produced, which has the 546S mutation and the (GGGGS)₂ linker. Previously produced plasmid p1512, is cut with PstI and PspX I, and CiP treated. The resulting 9739 bp sequence is retained and the 388 bp fragment is discarded. Previously produced plasmid p1461(FIG. 20) is cut with PstI and PspX I and the resulting 388 bp sequence is retained and the 9709 bp fragment is discarded. The retained sequences are treated under conditions allowing the retained sequences to join to form p1514 having the structure pTRAk-c-lph-ACE2(19-615,34S,546S)-(GGGGS)₂-hFc1

F. p1515 pTRAk-c-lph-ACE2(19-615,34V,546S)-(GGGGS)₂-hFc1

In this example F, the 34V mutation is introduced into previously produced vector p1512, which already has the 546S mutation and the (GGGGS)₂ linker. Previously produced p1512, is cut with PstI and PspX I, and CiP treated. The resulting 9739 bp sequence is retained and the 388 bp fragment is discarded. Plasmid p1468 is cut with Pst I and PspX I and the resulting 388 bp sequence is retained and, the 9709 bp fragment is discarded. The retained sequences are treated under conditions allowing the retained sequences to join to form p1515 having the structure pTRAk-c-lph-ACE2(19-615,34v,546S)-(GGGGS)₂-hFc1.

Vector: p1512, cut with Pst I and PspX I, CiP treat-keep 9739 bp, discard 388 bp. Insert: Cut the p1468 with Pst I and PspX I. Keep 388 bp, discard 9709 bp.

G. p1516 pTRAk-c-lph-ACE2(19-615,34S,546S, 273K)-(GGGGS)₂-hFc1

In this example G, the 34S mutation is introduced into a vector p1513, which already has the 546S mutation the 273K mutation and the (GGGGS)₂ linker. p1513 is cut with Pst I and PspX I and Cip treated. The resulting 9739 bp sequence is retained and the 338 bp fragment is discarded. Plasmid p1461 (shown in FIG. 20) is cut with PstI and PspXI. The 388 bp sequence is retained and the 9709 bp fragment is discarded. The retained sequences are treated under conditions allowing the retained sequences form for p1516 having the structure pTRAk-c-lph-ACE2(19-615,34S,546S, 273K)-(GGGGS)2-hFc1. Vector: p1513, cut with Pst I and PspX I, CiP treat-keep 9739 bp, discard 388 bp. Insert: Cut the p1461 with Pst I and PspX I. Keep 388 bp, discard 9709 bp.

H. p1517 pTRAk-c-lph-ACE2(19-615,34V,546S, 273K)-(GGGGS)2-hFc1

In this example H, the 34V mutation is introduced into the previously produced p1513, which already has the 546S mutation, the 273K mutation and the (GGGGS)₂ linker. p1513 is cut with Pst I and PspX I and Cip treated. The resulting 9739 bp sequence is retained and the 338 bp fragment is discarded. Previously produced plasmid p1468 is cut with Pst I and PspX I. The 388 bp sequence is retained and the 9709 bp fragment is discarded. The retained sequences are treated under conditions allowing the retained sequences to form p1517 having the structure pTRAk-c-lph-ACE2(19-615,34V,546S, 273K)-(GGGGS)2-hFc1. Vector: p1513, cut with Pst I and PspX I, CiP treat-keep 9739 bp, discard 388 bp. Insert: Cut the p1468 with Pst I and PspXI. Keep 388 bp, discard 9709 bp.

I. p1495 Modified ACE2(19-614,H34S)-Fc of IgG1

In this example I, a DNA sequence of nucleotides 1 to 1788 of SEQ ID NO: 3, which encodes the 595 amino acids corresponding to residues 19-614 of SEQ ID NO: 1) is modified to alter the codon CAC encoding residue H34 to TCC encoding residue S34 using primers 5′-TTCCTCGACAAGTTCAACGTGGAGGCCGAGGACCTCTTC (SEQ ID NO: 124) and 5′-GAAGAGGTCCTCGGCCTCCACGTTGAACTTGTCGAGGAA (SEQ ID NO: 125) by the method of overlap extension PCR essentially as described in Ho et al., “Site-directed mutagenesis by overlap extension using the polymerase chain reaction,” Gene. 1989; 77(1):S1-59. doi:10.1016/0378-1119(89)90358-2.

The modified DNA sequence encoding modified soluble ACE2 with amino acid S34 instead of the native H34 is then cloned into the pTRAkc plant binary vector (Maclean et al. 2007) in frame with an IgG1 Fc sequence optimized for expression in planta. Recombinant A. tumefaciens strains (GV3101::pMP90RK) carrying these expression vectors are used to transiently express this Modified ACE2-Fc in whole N. benthamiana plants (preferably DXT/FT) following vacuum-assisted agroinfiltration using known methods (Kapila et al. 1997; Vaquero et al. 1999). Co-infiltration of an additional A. tumefaciens strain (GV3101::pMP90RK) carrying the p19 silencing suppressor from tomato bushy stunt virus, is used to prevent post-transcriptional gene silencing and hence enhance expression levels (Voinnet et al. 2003). If terminal galactose residue are required a binary vector as described in Example 5 is co-infiltrated. The Agrobacterium cell suspensions are combined and diluted to appropriate concentrations in infiltration buffer. Whole N. benthamiana plants (3-6 plants per pot), inverted and submerged into the bacterial suspension, are subjected to two sequences of vacuum (to 20 in. Hg for 10 sec) followed by slow vacuum release (˜2 kPa/second) to draw the bacterial suspension into the spongy leaf interstitial space. Following infiltration, plants are grown for up to 8 days in a greenhouse. As described in greater detail herein in Example 2, the transfected plants are harvested, and plant juice is extracted by grinding in a Waring blender, the juice is separated by filtration and the protein is purified by Protein A chromatography.

J. p1496 Modified ACE2(H34S)-Fc of IgG₄

A DNA sequence encoding the soluble ACE2 sequence (596 amino acids corresponding to residues 19-614 of SEQ ID NO: 1) is modified to alter the codon CAC encoding residue H34 to TCC encoding residue S34 using primers 5′-TTCCTCGACAAGTTCAACGTGGAGGCCGAGGACCTCTTC (SEQ ID NO: 126) and 5′-GAAGAGGTCCTCGGCCTCCACGTTGAACTTGTCGAGGAA (SEQ ID NO: 127) by the method of overlap extension PCR as described above. The modified DNA sequence encoding modified soluble ACE2 with amino acid S34 instead of the native H34 is then cloned into the pTRAkc plant binary vector (Maclean et al. 2007) in frame with an IgG4 Fc sequence (Table 2, Gene Bank Accession No. K01316) which optionally is optimized for expression in planta. The complete amino sequence of the Modified ACE2(H34S)-Fc (IgG4) in-frame fusion of SEQ ID NO: 16 (shown in FIG. 13B). The corresponding DNA sequence is inserted in pTRAk plasmid (shown in FIG. 12) in the region denoted by ACE2 and Fc in the open reading frame (ORF). The ACE2(H34S)-Fc (IgG4) fusion protein is targeted to the plant cell secretory pathway via a signal peptide from a mouse antibody heavy chain. See FIG. 12 for plasmid maps for pTRAk-ACE2-Fc and pTRA-P19. Subsequent steps for vacuum infiltration and co-infiltration of N. benthamiana with either plasmid in Agrobacterium strains are as described above in Example 1 and Example 5. The further processing of the agroinfiltrated plants to produce the purified protein is as described in Example 2.

### Example 7: Selection of New ACE2-Fc Variants

The functionality of all new Modified ACE2-Fc variants is evaluated by binding to S1 protein of SARS-CoV-2 by ELISA as described in Example 3. The binding to S1 of the Modified ACE2-Fc variants is first evaluated to determine whether the mutation reduces binding. If the mutation does not reduce the binding to SARS-CoV-2 S1 it is further evaluated.

Each of the Modified ACE2 sequences is expressed transiently in the N. benthamiana, as described in Example 1. When expressed in wild type N. benthamiana with the pTRAkc vector lacking the proteins produced have wild type N-glycans. When expressed in N. benthamiana using KDEL-containing pTRAkc vector the proteins produced are high mannose of Example 5. When expressed in DXT/FT N. benthamiana with the pTRAkc vector lacking KDEL the proteins produced are N-glycan species without detectable plant-specific β1,2-xylose and α1,3-fucose residues. When expressed in DXT/FT N. benthamiana with the pTRAkc vector lacking KDEL previously transformed or co-transformed vector that encodes a modified human β1,4-galactosyl-transferase (ST-Gaff) the N-glycans are produced with terminal β1,4-Gal residues. The functionality of all new Modified ACE2-Fc variants is evaluated by binding to SARS-CoV-2 S1 protein using ELISA as described in Example 3. The binding of the Modified ACE2-Fc variants to SARS-CoV-2 S1 protein is first evaluated to determine whether the mutation reduces binding. If the mutation does not reduce the binding to SARS-CoV-2 S1 it is further evaluated.

A. Modified ACE2(19-614,H34S)-Fc of IgG1

A DNA sequence of SEQ ID NO: 3 (shown in FIG. 1C) encoding the soluble ACE2 sequence (595 amino acids corresponding to residues 19-614 of SEQ ID NO: 1, shown in FIG. 1A) is modified to alter the codon CAC encoding residue H34 to TCC encoding residue S34 using primers 5′-TTCCTCGACAAGTTCAACGTGGAGGCCGAGGACCTCTTC and 5′-GAAGAGGTCCTCGGCCTCCACGTTGAACTTGTCGAGGAA by the method of overlap extension PCR essentially as described in Ho S N, Hunt H D, Horton R M, Pullen J K, Pease L R. Site-directed mutagenesis by overlap extension using the polymerase chain reaction. Gene. 1989; 77(1):S1-59. doi:10.1016/0378-1119(89)90358-2.

The modified DNA sequence encoding modified soluble ACE2 with amino acid S34 instead of the native H34 is then cloned into the pTRAkc plant binary vector (Maclean et al. 2007) in frame with an IgG1 Fc sequence optimized for expression in planta. Recombinant A. tumefaciens strains (GV3101::pMP90RK) carrying these expression vectors are used to transiently express this Modified ACE2-Fc in whole N. benthamiana plants following vacuum-assisted agroinfiltration using known methods (Kapila et al. 1997; Vaquero et al. 1999). Co-infiltration of an additional A. tumefaciens strain (GV3101::pMP90RK) carrying the p19 silencing suppressor from tomato bushy stunt virus, is used to prevent post-transcriptional gene silencing and hence enhance expression levels (Voinnet et al. 2003). The Agrobacterium cell suspensions are combined and diluted to appropriate concentrations in infiltration buffer. Whole N. benthamiana plants (3-6 plants per pot), inverted and submerged into the bacterial suspension, are subjected to two sequences of vacuum (to 20 in. Hg for 10 sec) followed by slow vacuum release (˜2 kPa/second) to draw the bacterial suspension into the spongy leaf interstitial space. Following infiltration, plants are grown for up to 8 days in a greenhouse. As described in greater detail in Example 2, the transfected plants are harvested, and plant juice is extracted by grinding in a Waring blender, the juice is separated by filtration and the protein is purified by Protein A chromatography.

B. Modified ACE2(H34S)-Fc of IgG₄

A DNA sequence encoding the soluble ACE2 sequence (596 amino acids corresponding to residues 19-614 of SEQ ID NO: 1) is modified to alter the codon CAC encoding residue H34 to TCC encoding residue S34 using primers 5′-TTCCTCGACAAGTTCAACGTGGAGGCCGAGGACCTCTTC and 5′-GAAGAGGTCCTCGGCCTCCACGTTGAACTTGTCGAGGAA by the method of overlap extension PCR as described above. The modified DNA sequence encoding modified soluble ACE2 with amino acid S34 instead of the native H34 is then cloned into the pTRAkc plant binary vector (Maclean et al. 2007) in frame with an IgG4 Fc sequence optimized for expression in planta. The complete amino sequence of the H34S Modified ACE2 in-frame IgG4-Fc fusion is shown in FIG. 13B. The corresponding DNA sequence is inserted in pTRAk as shown in FIG. 12 in the region denoted by ACE2 and Fc in the open reading frame (ORF). In this example the expression construct lacked KDEL. The fusion protein is targeted to the plant cell secretory pathway via a signal peptide from a mouse antibody heavy chain. See FIG. 12, Plasmid maps for pTRAk-ACE2-Fc and pTRA-P19. Subsequent steps for vacuum infiltration and co-infiltration of N. benthamiana with either plasmid in Agrobaterium strains are as described above in Example 1 and the further processing of the agroinfiltrated plants to produce the purified protein is as described in Example 2.

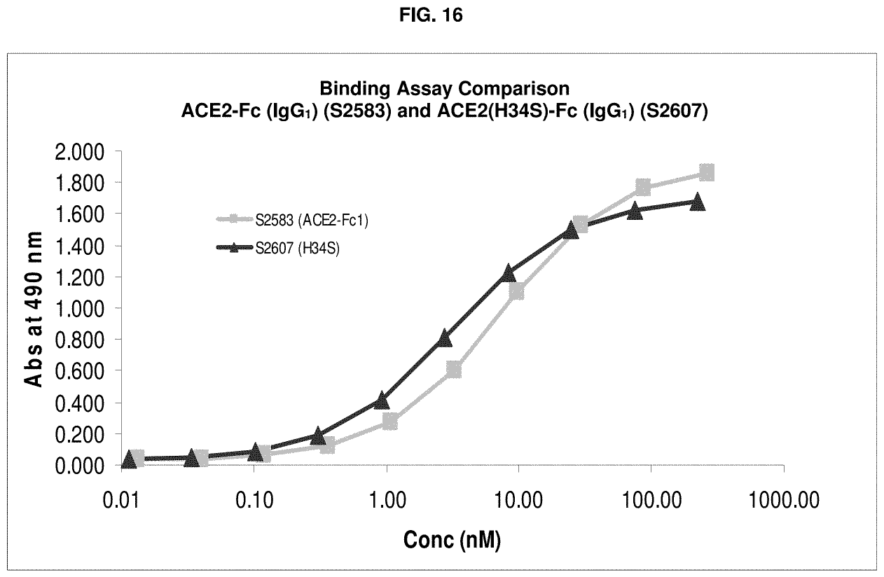

C. ELISA binding assay of ACE2(H34S)-Fc (IgG1): ELISA is performed as described above in Example 5 using the modified human ACE2 protein, ACE2(H34S)-Fc of IgG₁ having the sequence shown in FIG. 2B and ACE2-Fc of IgG1 having the sequence shown in FIG. 2A. As shown in the plots of FIG. 16, the binding affinity for S1 protein of SARS-CoV-2 of the ACE2(H34S)-Fc (IgG₁) is improved as compared to that of ACE2-Fc (IgG1).

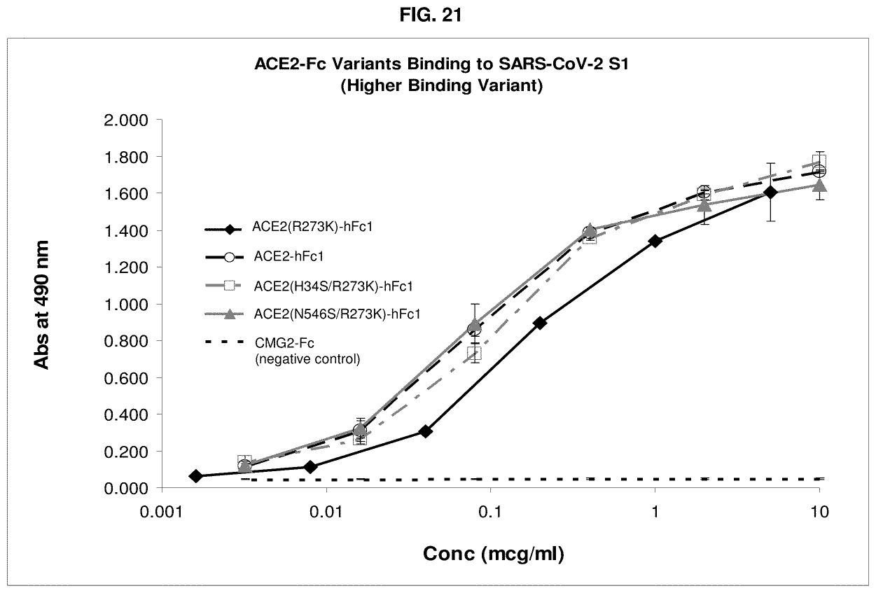

D. Simultaneous ELISA binding assay for modified variants: The ELISA binding assay for S1 protein of SARS-CoV-2 is performed as described above in Example 5 using the following variants produced as described in the Examples above: ACE2(19-614, R273K)-hFc1, (reference standard), ACE2(19-614)-hFc of IgG₁, ACE2(19-614, H34S, R273K)-hFc of IgG₁, ACE2(19-614, N546S, R273K)-hFc of IgG₁ and CMG2-hFc of IgG1 as a negative control for S1 binding. The graph of the ELISA assay results, showing the improvement in affinity of the variants ACE2(19-614, H34S, R273K)-hFc of IgG₁ and ACE2(19-614, N546S, R273K)-hFc of IgG₁ compared to both the reference standard and ACE2(19-614)-hFc of IgG₁ is shown in FIG. 21.

### Example 8: Measurement of ACE2-Fc and Modified ACE2-Fc Affinity for Recombinant SARS-CoV-2 S1 Spike Protein

Measurements of Affinity

Binding affinity is be determined with Bio Layer Interferometry (BLI) technology, utilizing the Octet 384 RED instrument (ForteBio, Fremont, Calif.). Assays are performed as follows (Bindkin Bio LLC, Davis, Calif.). The NiNTA biosensors (ForteBio, Fremont, Calif.) will be saturated with either terminally His-tagged SARS-CoV-2 S1 or SARS-CoV-2 RBD and subjected to a concentration series of ACE2::Fc or ACE2::Fc protein variants in 1× Kinetics Buffer containing 0.1% BSA (ForteBio, Fremont, Calif.). Briefly, after achieving a stable baseline (60-300 seconds), the association step are initiated (600 seconds), followed by the dissociation step (1200 seconds). Binding responses are continuously recorded. The concentration series is 5.0E-07, 3.3E-07, 2.2E-07, 1.5E-07, 9.9E-08, 6.6E-08, 4.4E-08, 2.9E-08, 2.0E-08, 1.3E-08, 8.7E-09, 5.8E-09, 3.9E-09, 2.6E-09, 1.7E-09, 1.1E-09 Molar. A 1:1 fitting model is utilized to fit the response traces, and the k_{obs}, k_{off}, kₒₙ and KD are determined by individual trace analysis provided by resident software supplied with the Octet384 RED instrument and integrative analysis. The integrative analysis is performed with a custom, proprietary Visual Basic for Applications (VBA) script, utilizing Microsoft Excel 2007 software. Briefly, the k_{obs} is plotted versus concentrations and fitted using the linear regression minimum sum of squares method. The k_{off} is calculated from this curve. The kₒₙ is determined from the k_{obs} and k_{off} via the equation k_{obs}=kₒₙ*Concentration+k_{off}. The efficacy (EC50) and Hill Slopes of binding is also determined using a 4-parameter model analysis of a Sigmoidal Dose Response plot derived from analysis of individual traces generated by the resident software supplied with the Octet384 RED instrument which reports and automatic EC50. The responses are plotted against the log of concentrations using a custom VBA script and Excel 2007. Next, the Sigmoidal dose response curve is fitted using the minimum sum of squares method and a model: Response=Rmin+(Rmax−Rmin)/(1+10\hat{ }((Log EC50−X)*HillSlope)); where X is the logarithm of concentration, Response starts at Rmin and goes to Rmax with a Sigmoidal shape. The interaction parameters are organized into an Excel table and are ranked and plotted onto an iso-affinity plot, where the kₒₙ of each interaction is graphed against the k_{off}, thus allowing the selection of ligands with desirable binding properties.

The calculated KD (nM) for each tested sample is found below in Table 7. The three ACE2-Fc fusion variants bound to SARS-CoV-2 RBD with affinities between 0.7 and 3.2 nM.

TABLE 7

| | Strain | | | | |
|---|---|---|---|---|---|
| | S2589 | S2585 | S2583 | | |
| | Protein | | | | |
| | ACE2-GS2- | ACE2-GS2- | ACE2- | | Neg |
| | hFc3 | hFc1 | hFc1 | rhACE2 | control |
| Biolayer | 0.7 | 0.8 | 3.2 | 6.7 | ND |
| Interferometry | | | | | |
| K_{D} (nM) | | | | | |

### Example 9: Viral Neutralization Assay

The potency of all ACE2-Fc variants was compared using an in vitro virus neutralization assay (VNA). Prior to running the VNA, the TCID₅₀ for the virus on Vero E6 cells which are susceptible to SARS-CoV-2 infection, is determined as follows. Aliquots of the virus are applied on confluent Vero-E6 cells in 96-well plates. Serial 10-fold dilutions of virus are inoculated in a Vero-E6 cell monolayer in quadruplicate and cultured in DMEM with 1% FBS and penicillin/streptomcycin. The plates are observed for cytopathic effects for 4 days. Viral titer is calculated with the Reed and Munch endpoint method (1), One TCID₅₀ is interpreted as the amount of virus that causes cytopathic effects in 50% of inoculated wells.

The VNA is performed using Vero E6 cells, under BSL-3 conditions. Vero E6 cells seeded into 96-well plates are incubated at 37° C. and 5% CO2 for one to three days until at least 80% confluency. On the day of assay, the ACE2-Fc variant is diluted with serum-free media to the desired starting concentration, 10 μg/ml, and then serially diluted 2 fold or 3-fold in serum-free media. To each well 100 50% tissue culture infective doses (TCID50) of virus is added and the mixture is incubated at 37° C. for 1 hour. The virus/ACE2-Fc mixture is then transferred into the Vero-seeded 96-well plates, which are then returned to the incubator for three to five days. Following incubation, using a phase contrast inverted scope, the wells are scored for the presence or absence of SARS-CoV-2 cytopathic effects (CPE). Neutralizing antibody titers (EC₅₀) are expressed as the concentration of ACE2-Fc that completely (100%) inhibits virus-induced CPE in at least 50% of the wells

Control wells include: 1) ACE2-Fc or ACE2-Fc variant at the highest concentration in the series without virus to ensure that the immunoadhesin itself does not cause CPE; 2) negative control wells (without ACE2-Fc or ACE2-Fc variant or virus) to verify that the serum-free media does not cause CPE; and 3) a back-titer of the virus, which verifies that the titer of the standardized virus is within acceptable range. If all controls meet their stated acceptance criteria, the results from the samples on that plate are considered valid. Results a shown below in Table 8. Three ACE2-Fc variants neutralized SARS-CoV-2 infection of Vero cells with EC₅₀ between 0.6 and 1.7 μg/ml.

TABLE 8

| | Strain | | | | |
|---|---|---|---|---|---|
| | S2589 | S2585 | S2583 | | |
| | Protein | | | | |
| | ACE2-GS2- | ACE2-GS2- | ACE2- | | Neg |
| | hFc3 | hFc1 | hFc1 | rhACE2 | control |
| in vitro virus | 1.7 | 0.6 | 0.9 | ND | >10 |
| neutralization | | | | | |
| EC₅₀ | | | | | |
| (μg/ml) | | | | | |
| cytotoxicity | >10 | >10 | >10 | ND | >10 |
| CC₅₀ | | | | | |

### Example 10: Effects of N. benthamiana line on ACE2 (19-614,H34S)-Fc1 and ACE2 (19-614,H34S)-Fc4 Binding to SARS-CoV-2 Spike 1 & Expression/Yield

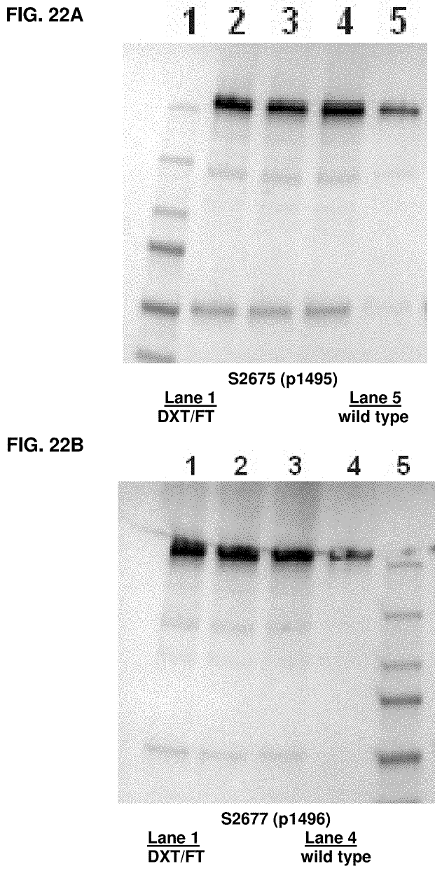

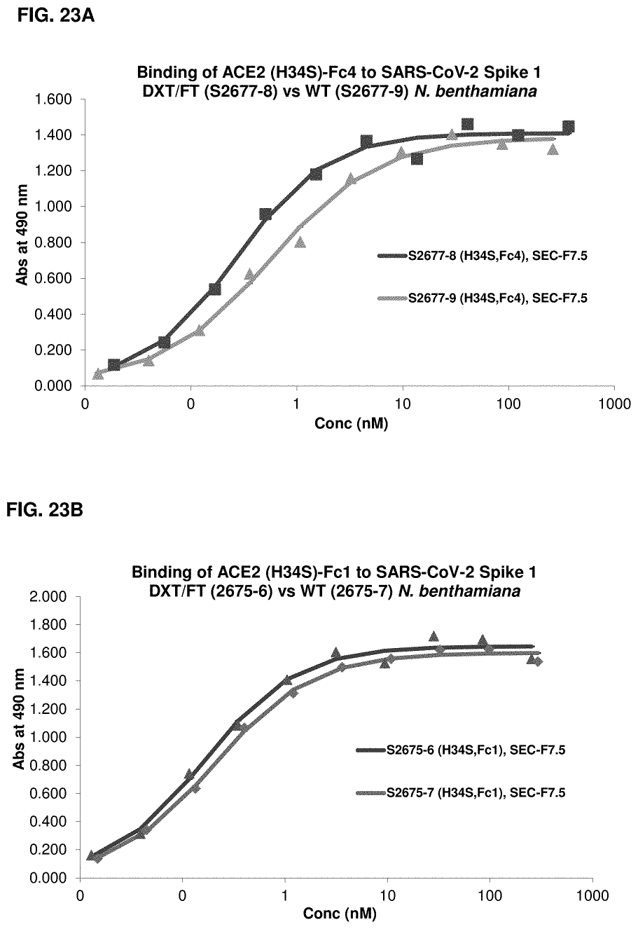

Agrobacterium strains 2675 and 2677 into which plasmid p1495 and p1496 respectively have been previously integrated were vacuum infiltrated and co-infiltrated with p19 into 38 day old whole N. benthamiana wild type or N. benthamiana DXT/FT plants as described above in Example 1 and Example 5 and subsequent further processing of the agroinfiltrated plants to produce the purified protein is as described in Example 2. Western Blots of the proteins produced from juice obtained in initial processing are shown in FIG. 22A and FIG. 22B. The purified protein was polished by size exclusion chromatography to provide a homogeneous sample. An ELISA binding assay was run as described in Example 5 for the purified glycoprotein produced by N. benthamiana Wild Type or DXT/FT. The results are shown in the plots depicted in FIG. 23A and FIG. 23B. The lower curve in each plot shows the assay for Wild Type and the upper curve shows the assay for DXT/FT. The results in Table 9 below summarize the calculated EC50 in mg/ml and yield in mg protein/gram fresh weight of plant biomass after protein A purification. ACE2-Fc expressed in wild type N. benthamiana appears to result in lower binding to SAR2-CoV-2 S1 and poorer expression in the plant as measured by protein A yield.

TABLE 9

| | | Protein A |
|---|---|---|
| | EC50 | Yield |
| | (mg/ml) | (mg/g FW) |
| Fc (IgG1) | | |
| S2675 (DXT/FT), F7.5 | 0.040 | 0.340 |
| S2675 (WT), F7.5 | 0.050 | 0.125 |
| Fc (IgG4) | | |
| S2677 (DXT/FT), F7.5 | 0.062 | 0.561 |
| S2677 (WT), F7.5 | 0.122 | 0.279 |

### Example 11: Effects of H34S mutation on ACE2-Fc binding to SARS-CoV-2 Spike S1 protein

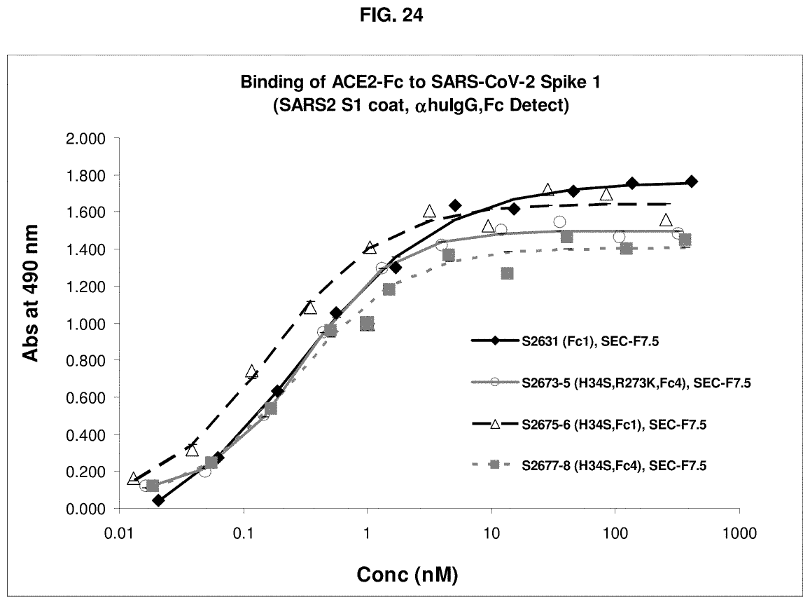

Agrobaterium strains 2631, 2673, 2675 and 2677 into which plasmids p1473, p1494, p1495 and p1496 respectively have been previously integrated, were vacuum infiltrated and co-infiltrated with p19 into N. benthamiana Wild Type or N. benthamiana DXT/FT plants as described above in Example 1 and Example 5 and subsequent further processing of the agroinfiltrated plants to produce the purified protein is as described in Example 2. The purified protein was polished by size exclusion chromatography to provide a homogeneous sample. An ELISA Assay was run as described in Example 5 for the purified glycoprotein produced by N. benthamiana Wild Type or DXT/FT. The results are shown in the plot depicted in FIG. 24 and summarized in Table 10 below.

TABLE 10

| | EC50 |
|---|---|
| ACE2-Fc fusion protein | (mcg/ml) |
| S2631, F7.5 ACE2(19-614)-huFc1 | 0.083 |
| S2673-5, F7.5 ACE2(19-614, H34S/R273K)-huFc1 | 0.062 |
| S2675-6, F7.5 ACE2(19-614, H34S)-huFc1 | 0.040 |
| S2677-8, F7.5 ACE2(19-614, H34S)-huFc4 | 0.062 |

The three purified ACE2-Fc fusion proteins in which the huACE2 sequence corresponding to amino acids 19-614 of SEQ ID NO: 1 has the H34S amino acid change, have improved EC50 values compared to the same ACE2 sequence lacking the mutation.

### Example 12: Assay of ACE2 Carboxypeptidase Activity

The assay for ACE2 carboxypeptidase activity is based on the ability of ACE2 to cleave the fluorogenic substrate (FS), Mca-APK-Dnp (Anaspec). Cleavage of FS by ACE2 removes the moiety that quenches the fluorescence, thus resulting in increased fluorescence. The cleavage assay was carried out by serially diluting purified samples of rhACE2 and the three ACE2-Fc fusions indicated in the figure, using assay buffer (50 mM MES, 300 mM NaCl, 10 uM ZnSO4, pH 6.5). The substrate for the assay, 2.25 mM Mca-APK(Dnp)-OH (200× stock) is diluted to 2× using assay buffer. 50 ul of 2× substrate is added per well of a 96-well plate. Into each well, 50 ul of diluted sample is added and read immediately using a microplate via a kinetic read using excitation 320/20, emission 400/30 for 1 hr. The slope is determined using the linear portion of the kinetic curve to provide a RFU/min. Lastly, the RFU/min is divided by the amount of sample added to the well to generate the RFU/min/ug.

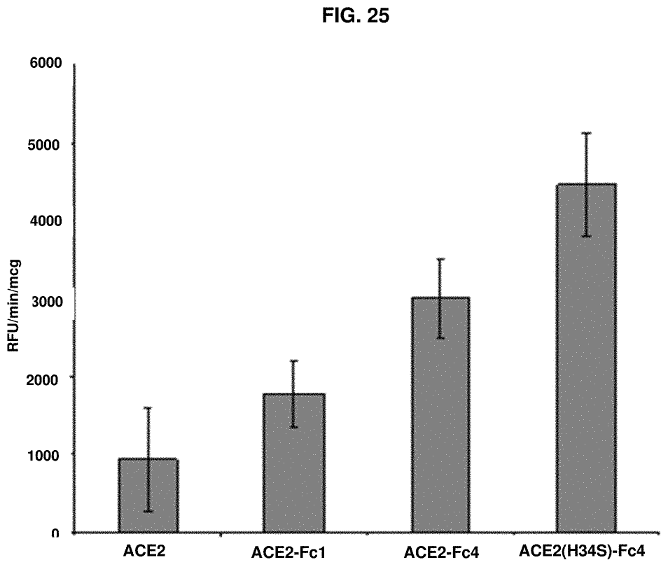

As shown by the plot depicted in FIG. 25, ACE2-Fc in which the Fc is that of IgG4 has increased ACE2 carboxypeptidase activity compared to ACE2-Fc in which the FC is that of IgG1 Fc. In addition, the H34S mutation of the ACE2 sequence further increased ACE2 carboxypeptidase activity the ACE2-Fc in which the Fc is that of IgG4. The plant-made ACE2-Fc4 has 5-fold greater ACE2 carboxypeptidase activity (on a weight basis) than recombinant soluble human ACE2 (purchased from Acro Biosystems). It is expected that administration of ACE2-Fc whether the Fc is that of IgG1 or IgG4 may require less protein than would administration with rhACE2 to achieve equivalent reductions in serum angiotensin II and increases in serum angiotensin 1-7. In addition, the circulating half-life of ACE2-Fc is expected to be greater compared to rhACE2.

### Example 13: In Vivo Administration of ACE2-Fc

ACE2-Fc is tested in the hamster model against SARS-CoV-2 virus strain WA1/2020 representative of the etiologic agent of the COVID-19 global pandemic as follows.

At the time of testing animals are 6 to 7 weeks of age and are randomized into study groups by weight with each group containing equal number of males and females. 24 hours prior to treatment with ACE2-Fc, animals are challenged with 10⁵ TCID₅₀ (or 10⁵ PFU) of SARS-CoV-2 virus strain WA1/2020 by intranasal instillation in a volume of 100 microliters (50 μI into each nare) of the test animal. On study days 1 and 3 a total of 200 microliters (100 microliters/nare) of either ACE2-Fc (delivering 0.6 mg/kg) or PBS is administered to anesthetized test and control animals respectively. Hamsters are observed twice daily for clinical signs of infection and respiratory rate. A scoring system is applied to summarize respiratory quality. Body weights, clinical observations, respiratory rate and respiratory quality scoring are collected daily post challenge through the end of the 7-day study. To assess viral load in the respiratory tract, nasal washes are performed on days 2 and 4 post challenge as follows, 300 microlitres of PBS are instilled in each nostril (i.e. 600 microlitres per animal per assessment) and then collected for analysis using a TCID₅₀ assay on VERO E6 cells in culture. Blood is collected from all animals via retro orbital bleeding on days 2 and 4 of the study. A terminal blood collection is performed on animals sacrificed on day 7 of the study.

On study day 7 all animals are euthanized and the left or right lung lobe is perfused with 10% buffered formalin before being submerged in a 10% formalin solution. The other lobe is frozen and stored for future analysis. Any additional organs that appear abnormal will be collected and preserved in 10% buffered formalin. Statistical comparison between the 8 treated and 4 vehicle control animals will be made on survival, body weight, respiratory rate and quality score, histopathology, tissue viral titers and clinical observations to demonstrate efficacy.

ACE2-Fc Quantification Assay

Quantification of ACE2-Fc and ACE2 variant Fc exhibiting S protein binding activity in serum, BAL and/or homogenized lung tissue is performed by ELISA. Ninety-six-well ELISA plates will be coated with the S1 domain or RBD (Sino Biological). After blocking with 5% non-fat dry milk in buffer, serially diluted serum or lung homogenates containing ACE2-Fc, or variant, is added. Samples from control animals spiked with purified ACE2-Fc or variant as the case may be, are used to generate standard curves. After washing, a peroxidase-conjugated anti-human IgG Ab is added to detect bound ACE2-Fc. Lastly, OPD substrate in citrate buffer is used to provide a colorimetric signal and read at 490 nm via a Synergy™ HT Multi-Detection Microplate Reader (BioTek Instruments). Absorbance values are plotted and fit to a 4-parameter logistic model (GraphPad, San Diego, Calif.). Preliminarily, the lower limit of quantitation of this assay is −15 ng/mL. The parameters of the study design is summarized in the Table 11 below.

TABLE 11

| | | | | Day of Nasal | | | | |
|---|---|---|---|---|---|---|---|---|
| Group | No. of | Test | | Wash and | Necropsy | Challenge | Challenge | Dosing |
| No. | Animals | Article | Route | RO Bleed | Day | Virus | Day | Day |
| 1 | 4 | PBS | IN | 2 & 4 | 7 | SARS- | 0 | 1 & 3 |
| 2 | 8 | ACE2-Fc | | | | COV-2 | | |
| | | | | | | WA1/2020 | | |

### Example 14: Nebulization of ACE2-Fc

The effect of nebulization on ACE2-Fc aggregation, binding to SARS-CoV-2 Spike 1, and enzymatic activity of ACE2 was determined using a vibrating mesh nebulizer Aerogen® Solo (Aerogen Ltd., Galway Ireland) to deposit nebulized ACE2-Fc in silanized glass tube having opening size corresponding to the outlet side of the nebulizer.

Commercially silanized glassware are available through various sources; however in this example borosilicate glass tubes were treatment with a silanizing agent Jersey-Cote (Lab Scientific, Cat#1188) The entire inside of the glass tube was treated by adding the silanizing agent up to the top of the tube, completely decanting the the silanizing agent, and inverting the tubes. which were allowed to dry overnight followed by multiple rinses with deionized water before use (water should bead on the silanized surface).

Nebulization of ACE2-Fc

The silanized glass tube is positioned with its opening surrounding the outlet of the nebulizer and in contact with the nebulizer to avoid loss of sample through gaps at the interface. Assemble the nebulizer according to the manufactures instructions and load the nebulizer with 0.05-0.2 ml of the ACE2-Fc to be tested in buffer solution (0.6-25 mg/ml tested). Run the nebulizer until as much as possible of inlet volume has gone through.. The nebulized sample is allowed to condense on the collection tube for a few minutes before disconnecting the nebulizer from the tube. The collection tubes are centrifuged in a swing bucket rotor for 5 mins at 1000-4000 RPM after which the nebulized ACE2-Fc sample is for analysed.

HPLC-SEC analysis of nebulized ACE2-Fc

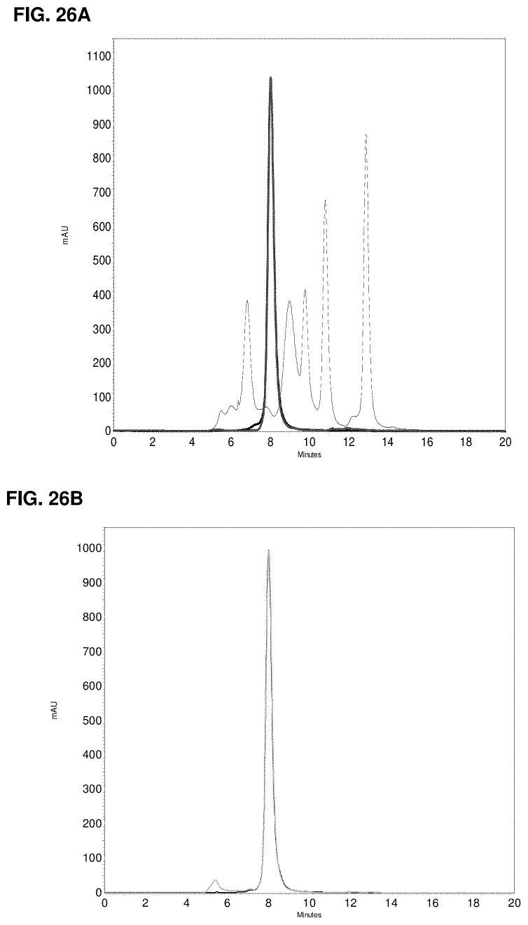

The Nebulized ACE2-Fc produced above is analyzed for formation of aggregates by HPLC using a BioSep-SEC-s3000 column, running buffer of 1×PBS at a rate of 1 ml/min for 20 min. The HPLC traces are shown in FIG. 26A and FIG. 26B. For both the ACE2-Fc1 and ACE2-Fc4 fusions, the HPLC traces were nearly identical before and after nebulization indicating that no significant multimerization or fragmentation had occurred.

SARS-CoV-2 Spike 1 Binding Analysis of Nebulized ACE2-Fc

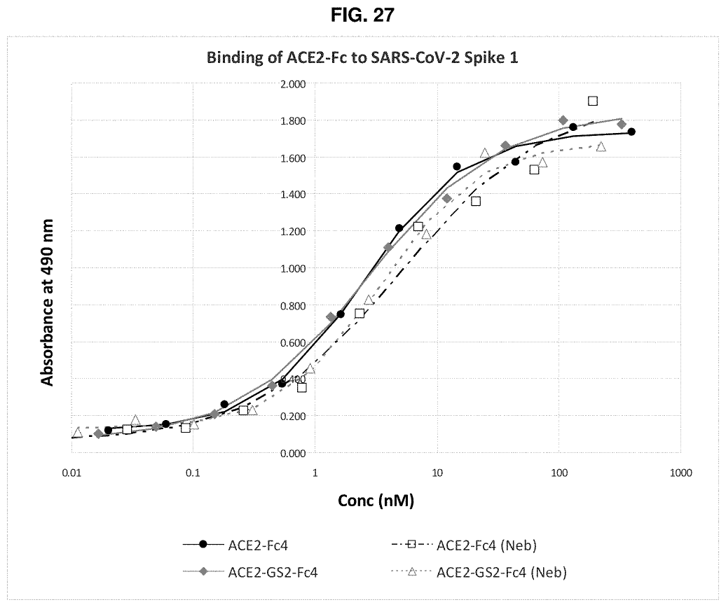

Following nebulization, binding of ACE2-(19-614)-Fc4 and ACE2-(19-615,H34S)-(GGGGS)2-Fc4 were analyzed by ELISA and compared with corresponding non-nebulized control sample at the same concentration. Plates were coated with SARS-Cov-2 Spike 1 protein (Wuhan) and detected with Goat anti-hu-IgG-HRP as described in Example 5. As shown by the plots depicted in FIG. 27, nebulization did not impair the binding of either fusion protein to the SARS-Cov-2 Spike 1 protein.

Enzymatic Activity Analysis of Nebulized ACE2-Fc

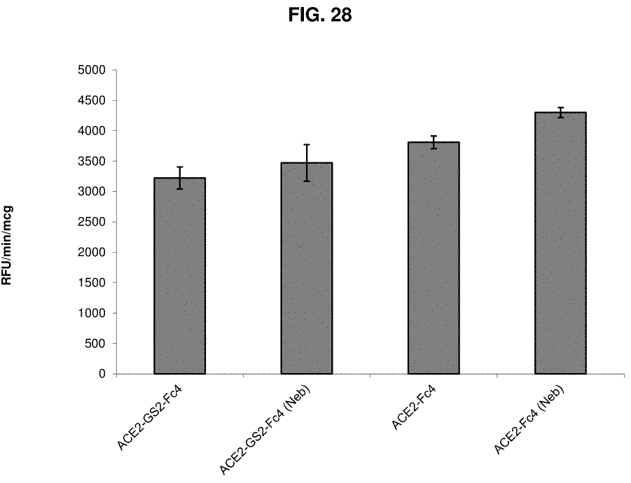

The same nebulized products and corresponding non-nebulized controls described in the ELISA binding assay were analyzed for the effect of nebulization on enzymatic activity. The assay was run as described in Example 10. As shown by the plots depicted in FIG. 28, there was no significant loss of enzymatic activity observed in the nebulized ACE2-Fc fusion proteins relative to the non-nebulized samples.

### Example 15: ELISA assays comparing ACE2, ACE2-(19-614)-Fc4, ACE2-(19-614,H34S)-Fc4, and ACE2-(19-614,H34S)-Fc4-Gal binding to the Spike Protein S1 from SARS-CoV-2 Wuhan and the SARS-CoV-2 variants UK, Mink, South Africa and UK Plus

To demonstrate the ability of ACE2-Fc immunoadhesins to maintain binding to emerging SARS-CoV-2 variant viruses that have evolved with modifications to the S1 protein and may show decreased binding to monoclonal antibodies or polyclonal antibodies derived from patients previously infected with the Wuhan Strain or who have been previously vaccinated—so-called escape mutants—soluble ACE2 (aa 19-614 of SEQ ID NO: 1), ACE2-(19-614)-Fc4, ACE2(19-614,H34S)-Fc4 and ACE2(19-614,H34S)-Fc4-Gal were assayed for their ability to maintain binding to S1 Spike protein variants having the mutations as described in Table 12 below.

TABLE 12

| S1 Variant (Full length spike) | Mutations |
|---|---|
| Wuhan | Wild type |
| UK (B.1.1.7) | HV69-70 deletion, N501Y, D614G |
| Mink | HV69-70 deletion, Y453F, D614G |
| South Africa (B.1.351) | K417N, E484K, N501Y, D614G |
| UK+ (B.1.1.7+) | HV69-70 deletion, Y144del, |
| | N501Y, A570D, D614G, P681H |

The S1 Spike protein variants were purchased from Sino Biological US, Inc. and the ELISA was carried as described in the Table 13 below.

TABLE 13

| ELISA Procedure: | Incubation |
|---|---|
| Coat: SARS-CoV-2 S1 variants | 60′, 37 C. |
| (Sino Biological, 2 mcg/ml in 1xPBS) | |
| Block: 5% Non-Fat Dry Milk in 1xPBS | 15′, 27 C. |
| Sample: Various ACE2 proteins | 60′, 37 C. |
| Primary Ab: rabbit anti-ACE2 | 60′, 37 C. |
| (ProSci Inc., 0.3 mcg/ml) | |
| 2nd Ab: goat anti-rabbit-HRP | 60′, 37 C. |
| (Jackson Lab, 0.5 mcg/ml) | |
| Develop w/OPD/Citrate/H₂O₂ | |
| for 20 min at Room Temp. | |

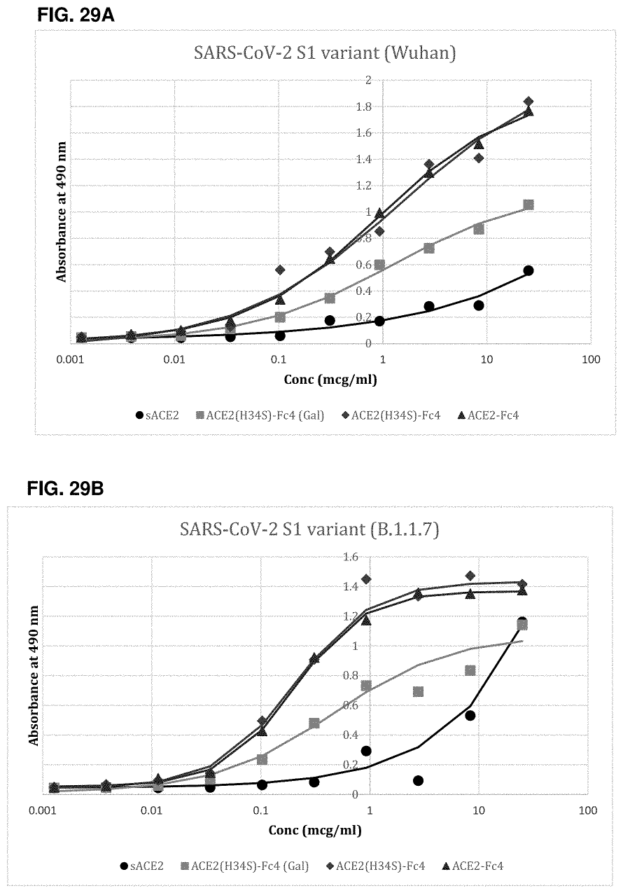

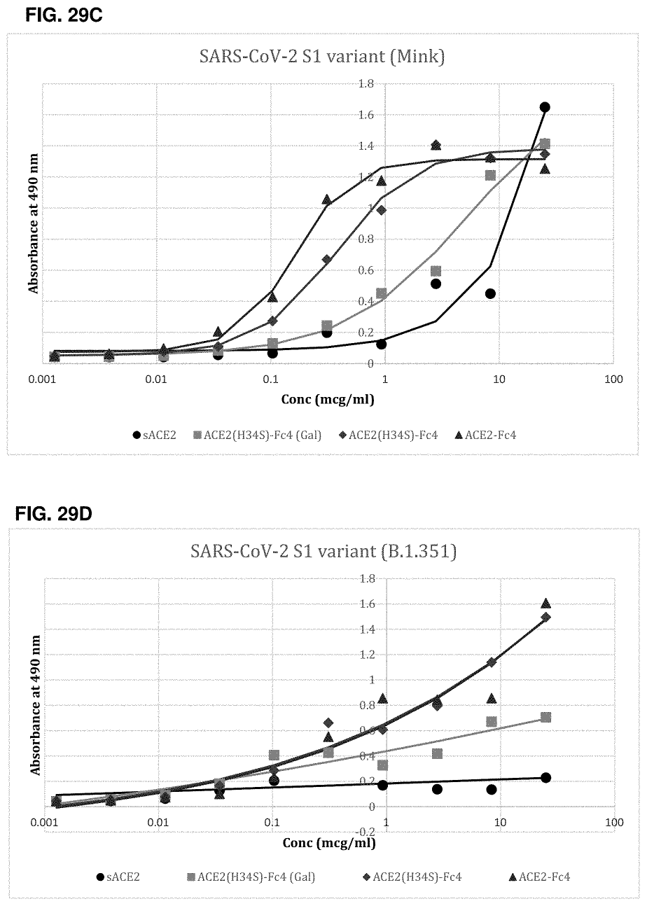

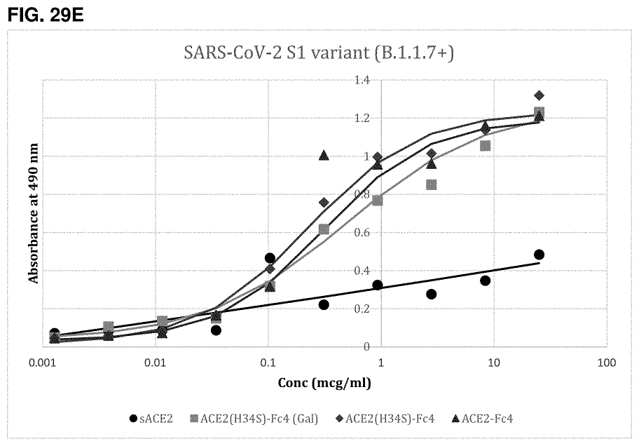

Binding curves were plotted (as shown in FIGS. 29A-29E) for the Soluble ACE2, ACE2-(19-614)-Fc4, ACE2(19-614, H34S)-Fc4 and ACE2(19-614, H34S)-Fc4-Gal, and are labeled “Soluble ACE2,” “ACE2-Fc4,” “ACE2(H34S)-Fc4” and “ACE2(H34S)-Fc4-Gal,” respectively against the Spike Protein S1 variants as indicated in the plots. In general, as shown by the binding curves shown in FIGS. 29A-29E, the binding of ACE2-(19-614)-Fc4 and ACE2(19-614, H34S)-Fc4 to each of the Spike protein S1 variants tested are similar to one another and greater than binding of ACE2(19-614, H34S)-Fc4-Gal which is greater than Soluble ACE2.

Notwithstanding the appended claims, the disclosure set forth herein is also defined by the following clauses, which may be beneficial alone or in combination, with one or more other causes or embodiments. Without limiting the foregoing description, certain non-limiting clauses of the disclosure numbered as below are provided, wherein each of the individually numbered clauses may be used or combined with any of the preceding or following clauses. Thus, this is intended to provide support for all such combinations and is not necessarily limited to specific combinations explicitly provided below:
1. A modified human ACE2 protein comprising at least amino acids 19-614 of the human ACE2 protein sequence of SEQ ID NO: 1 with at least one consensus contact sequence residue altered relative to SEQ ID NO: 1, wherein affinity of the modified human ACE2 protein for the S1 spike protein of SARS-CoV-2 is increased relative to affinity of the human ACE2 protein of SEQ ID NO: 1 for the S1 spike protein of SARS CoV-2.
2. The ACE2 protein of clause 1, wherein said at least one altered residue is selected from amino acid residues 30-42.
3. The ACE2 protein of clause 2, wherein said at least one altered residue is selected from amino acid residues 30, 31, 34, 38, 40, and 41.
4. The ACE2 protein of clause 3, wherein said at least one altered residue comprises an amino acid change selected from D30E, D30S, K31Q, K31E, H34S, H34V, D38E, F40S, Q42A, and combinations thereof; optionally, wherein the at least one residue altered comprises the two amino acid changes D38E and F42S.
5. The ACE2 protein of any one of clauses 1-4, wherein said at least one altered residue is selected from amino acid residues 81-84.
6. The ACE2 protein of clause 5, wherein said at least one altered residue is selected from amino acids 81 and 82.
7. The ACE2 protein of clause 6, wherein said at least one altered residue comprises an amino acid change selected from Q81K, M82N, M82K, M82T, and combinations thereof; optionally wherein the at least one altered residue comprises at least the two amino acid changes Q81K and M82N.
8. The ACE2 protein of any one of clauses 1-7, wherein said at least one altered residue is selected from amino acid residues 353-357.
9. The ACE2 protein of clause 8, wherein said at least one altered residue comprises an amino acid change selected from G354H or G354K.
10. The ACE2 protein of any one of clauses 1-9, wherein said at least one residue altered is selected from amino acid residues 327-329.
11. The ACE2 protein of clause 10, wherein said at least one residue altered comprises an amino acid change selected from E329N or E329K.
12. The ACE2 protein of any one of clauses 1-11, wherein at least one N-glycosylation site residue is changed from an N to an amino acid residue that does not glycosylate; optionally, wherein the at least one N-glycosylation site is changed from an N to S.
13. The ACE2 protein of clause 12, wherein the N-glycosylation site residue at position 546 has been changed from N to S.
14. The ACE2 protein of any one of clauses 1-13, wherein an amino acid residue adjacent to an N-glycosylation site residue is changed to an amino acid residue that prevents glycosylation at the adjacent N-glycosylation site.
15. The ACE2 protein of clause 14, wherein the amino acid residue at position 547 adjacent to the N-glycosylation site at position 546 is changed from S to P.
16. The ACE2 protein of any one of clauses 13-15, further comprising an amino acid change selected from H34S and H34V.
17. The ACE2 protein of any one of clauses 1-16 further comprising a fusion with a human immunoglobulin Fc region.
18. The ACE2 protein of clause 17, wherein said immunoglobulin is selected from the group consisting of IgG1, IgG2, IgG3, IgG4, IgA1, IgA2, IgM, IgD, and IgE; optionally, wherein the human immunoglobulin Fc region comprises an amino acid sequence of any one of SEQ ID NOs: 18-22.
19. The ACE2 protein of clause 18, wherein said immunoglobulin is IgG1; optionally, wherein the ACE2 protein fusion with the Fc region comprises an amino acid sequence of SEQ ID NO: 5.
20. The ACE2 protein of clause 18, wherein said immunoglobulin is IgG4; optionally, wherein the ACE2 protein fusion with the Fc region comprises an amino acid sequence of SEQ ID NO: 15.
21. The ACE2 protein of any one of clauses 17-20, wherein said Fc region further comprises a KDEL sequence at the carboxy terminus thereof.
22. The ACE2 protein of any one of clauses 17-21, wherein said fusion comprises said Fc region amino terminus linked to said ACE2 protein carboxy terminus.
23. The ACE2 protein of any one of clauses 17-21, wherein said fusion comprises said Fc region carboxy terminus linked to said ACE2 protein amino terminus.
24. The ACE2 protein of any one of clauses 17-23, wherein said fusion comprises a linker;
optionally, wherein the linker comprises an amino acid sequence of any one of SEQ ID NO: 23-49.
25. The ACE2 protein of any one of clauses 1-24, wherein the protein has typical mammalian glycosylation.
26. The ACE2 protein of any one of clauses 1-25, wherein N-glycans of said protein lack detectable β1,2-xylose and α1,3-fucose residues.
27. The ACE2 protein of clause 26, wherein the protein comprises a terminal β1,4-Gal residue at an N-glycan.
28. The ACE2 protein of any one of clauses 26-27, wherein the protein is produced in a DXT/FT plant; optionally, wherein the plant is N. benthamiana.
29. The ACE2 protein of clause 28, wherein the DXT/FT plant is modified to add terminal p1,4-Gal residues to N-glycan protein; optionally, wherein the modification comprises prior infiltration or co-infiltration with a binary vector encoding a human β1,4-galactosyl-transferase (ST-Gaff).
30. The ACE2 protein of any one of clauses 26-29, wherein the ACE2 protein is humanized.
31. The ACE2 protein of any one of clauses 1-30 wherein the ACE2 protein is enzymatically active.
32. The ACE2 protein of any one of clauses 1-30 wherein the ACE2 protein is enzymatically inactive.
33. A nucleic acid sequence encoding an ACE2 protein of any one of clauses 1-32.
34. An expression vector comprising the nucleic acid sequence of clause 33.
35. A chimeric SARS-CoV-2 S1 spike protein receptor comprising: an immunoglobulin complex, wherein the immunoglobulin complex comprises at least a portion of an immunoglobulin heavy chain, and a modified ACE2 protein linked to the immunoglobulin heavy chain, wherein the modified ACE2 protein comprises at least amino acids 19-614 of the human ACE2 protein sequence of SEQ ID NO: 1 with at least one consensus contact sequence residue altered relative to SEQ ID NO: 1 to increase affinity for the SARS-Cov-2 S1 spike protein.
36. The chimeric receptor of clause 35, wherein the immunoglobulin complex further comprises at least a portion of an immunoglobulin light chain; optionally, wherein portion comprises a kappa chain or a lambda chain.
37. The chimeric receptor of any one of clauses 35-36, wherein the linkage between the modified ACE2 protein and the immunoglobulin heavy chain comprises an immunoglobulin hinge.
38. The chimeric receptor of any one of clauses 35-37, wherein the immunoglobulin heavy chain is from an immunoglobulin selected from the group consisting of IgG1, IgG2, IgG3, IgG4, IgA1, IgA2, IgM, IgD, and IgE; optionally, wherein the human immunoglobulin heavy chain comprises an amino acid sequence of any one of SEQ ID NOs: 18-22.
39. The chimeric receptor of any one of clauses 35-38, wherein the immunoglobulin heavy chain is from an IgG and comprises heavy chain constant regions 2 and 3 thereof.
40. The chimeric receptor of any one of clauses 35-39, wherein the immunoglobulin heavy chain is from an IgA and comprises heavy chain constant regions 2 and 3 thereof.
41. The chimeric receptor of any one of clauses 35-40, wherein the immunoglobulin heavy chain and the modified ACE2 protein are human proteins.
42. A composition comprising the chimeric SARS-CoV-2 S1 spike protein receptor of any one of clauses 35-41 and plant material.
43. The composition of clause 42, wherein the plant material is selected from the group consisting of: plant cell walls, plant organelles, plant cytoplasm, intact plant cells, plant seeds, and viable plants.
44. A method for reducing binding of SARS-CoV-2 to a host cell, the method comprising: contacting the SARS-CoV-2 with the chimeric SARS-CoV-2 S1 spike protein receptor of any one of clauses 32-38, wherein the chimeric receptor binds to the SARS-CoV-2 Receptor Binding Domain (RBD) thereby reducing the binding of SARS-CoV-2 to the host cell.
45. A chimeric SARS-CoV-2 S1 spike protein receptor of any one of clauses 35-41 for use as a medicament.
46. A chimeric SARS-CoV-2 S1 spike protein receptor of any one of clauses 35-41 for use in preventing or treating SARS-CoV-1 infection, or the effects thereof.
47. An expression vector encoding a chimeric SARS-CoV-2 S1 spike protein receptor of any one of clauses 35-41.
48. A method for producing a chimeric SARS-CoV-2 S1 spike protein receptor of any one of clauses 35-41, comprising introducing an expression vector of clause 44 into a cellular host and expressing the immunoglobulin complex and the ACE2 peptide to form the chimeric ACE2 peptide.
49. The method of clause 48, wherein the host is a plant.
50. A pharmaceutical composition comprising a chimeric SARS-CoV-2 S1 spike protein receptor as in any one of clauses 35-41 and a pharmaceutically acceptable carrier.
51. A method for producing a modified human ACE2 protein of any one of clauses 1-32, comprising introducing an expression vector of clause 31 into a cellular host; and expressing the ACE2 protein.
52. The method of clause 51, wherein the host is a plant.
53. A pharmaceutical composition comprising a modified human ACE2 protein of any one of clauses 1-32 and a pharmaceutically acceptable carrier.
54. A method for reducing binding of SARS-CoV-2 to a cell, the method comprising: contacting the SARS-CoV-2 with the modified human ACE2 protein of any one of clauses 1-32, wherein the modified human ACE2 protein binds to the SARS-CoV-2 Receptor Binding Domain (RBD), thereby reducing the binding of SARS-CoV-2 to the cell.
55. A modified human ACE2 protein of any one of clauses 1-32 for use as a medicament.
56. The ACE2 protein of clause 55 provided in a nebulizer.
57. The modified human ACE2 protein of any one of clauses 1-32 for use in treating or preventing a SARS-CoV-2 infection, or the effects thereof.

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the invention are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

### REFERENCES

Boehm, M. K., J. M. Woof, M. A. Kerr, and S. J. Perkins. 1999. ‘The Fab and Fc fragments of IgA1 exhibit a different arrangement from that in IgG: a study by X-ray and neutron solution scattering and homology modelling’, J Mol Biol, 286: 1421-47.
Boehm, M., and E. G. Nabel. 2002. ‘Angiotensin-converting enzyme 2—a new cardiac regulator’, N Engl J Med, 347: 1795-7.
Brunke, C., S. Lohse, S. Derer, M. Peipp, P. Boross, C. Kellner, T. Beyer, M. Dechant, L. Royle, L. P. Liew, J. H. Leusen, and T. Valerius. 2013. ‘Effect of a tail piece cysteine deletion on biochemical and functional properties of an epidermal growth factor receptor-directed IgA2 m (1) antibody’, MAbs, 5: 936-45.
Casasnovas, J. M., and T. A. Springer. 1994. ‘Pathway of rhinovirus disruption by soluble intercellular adhesion molecule 1 (ICAM-1): an intermediate in which ICAM-1 is bound and RNA is released’, J Virol, 68: 5882-9.
Donoghue, M., F. Hsieh, E. Baronas, K. Godbout, M. Gosselin, N. Stagliano, M. Donovan, B. Woolf, K. Robison, R. Jeyaseelan, R. E. Breitbart, and S. Acton. 2000. ‘A novel angiotensin-converting enzyme-related carboxypeptidase (ACE2) converts angiotensin I to angiotensin 1-9’, Circ Res, 87: E1-9.
Eryilmaz, E., A. Janda, J. Kim, R. J. Cordero, D. Cowburn, and A. Casadevall. 2013. ‘Global structures of IgG isotypes expressing identical variable regions’, Mol Immunol, 56: 588-98.
Furtado, P. B., P. W. Whitty, A. Robertson, J. T. Eaton, A. Almogren, M. A. Kerr, J. M. Woof, and S. J. Perkins. 2004. ‘Solution structure determination of monomeric human IgA2 by X-ray and neutron scattering, analytical ultracentrifugation and constrained modelling: a comparison with monomeric human IgA1’, J Mol Biol, 338: 921-41.
Goebl, N. A., C. M. Babbey, A. Datta-Mannan, D. R. Witcher, V. J. Wroblewski, and K. W. Dunn. 2008. ‘Neonatal Fc receptor mediates internalization of Fc in transfected human endothelial cells’, Mol Biol Cell, 19: 5490-505.
Goetze, A. M., Y. D. Liu, Z. Zhang, B. Shah, E. Lee, P. V. Bondarenko, and G. C. Flynn. 2011. ‘High-mannose glycans on the Fc region of therapeutic IgG antibodies increase serum clearance in humans’, Glycobiology, 21: 949-59.
Goldblum, R. M., and R. P. Garofolo. 2004. ‘The mucosal defense system.’ in R. E. Stiehm, H. D. Ochs and J. A. Winkelstein (eds.), Immunologic disorders in Infants & Children (Elsevier: Philadelphia, Pa.).
Guy, J. L., R. M. Jackson, H. A. Jensen, N. M. Hooper, and A. J. Turner. 2005. ‘Identification of critical active-site residues in angiotensin-converting enzyme-2 (ACE2) by site-directed mutagenesis’, Febs J, 272: 3512-20.
Hadlington, J. L., A. Santoro, J. Nuttall, J. Denecke, J. K. Ma, A. Vitale, and L. Frigerio. 2003. ‘The C-terminal extension of a hybrid immunoglobulin A/G heavy chain is responsible for its Golgi-mediated sorting to the vacuole’, Mol Biol Cell, 14: 2592-602.
Han, D. P., A. Penn-Nicholson, and M. W. Cho. 2006. ‘Identification of critical determinants on ACE2 for SARS-CoV entry and development of a potent entry inhibitor’, Virology, 350: 15-25.
Hand, W. L., and J. R. Cantey. 1974. ‘Antibacterial mechanisms of the lower respiratory tract. I. Immunoglobulin synthesis and secretion’, J Clin Invest, 53: 354-62.
Kanda, Y., T. Yamada, K. Mori, A. Okazaki, M. Inoue, K. Kitajima-Miyama, R. Kuni-Kamochi, R. Nakano, K. Yano, S. Kakita, K. shitara, and M. Satoh. 2007. ‘Comparison of biological activity among nonfucosylated therapeutic IgG1 antibodies with three different N-linked Fc oligosaccharides: the high-mannose, hybrid, and complex types’, Glycobiology, 17: 104-18.
Kapila, J., R De Rycke, M. M. Van Monatagu, and G. Angenon. 1997. ‘An Agrobacterium-mediated transient gene expression system for intact leaves’, Plant Science, 122: 101-08.
Keidar, S., M. Kaplan, and A. Gamliel-Lazarovich. 2007. ‘ACE2 of the heart: From angiotensin I to angiotensin (1-7)’, Cardiovasc Res, 73: 463-9.
Kuba, K., Y. Imai, S. Rao, H. Gao, F. Guo, B. Guan, Y. Huan, P. Yang, Y. Zhang, W. Deng, L. Bao, B. Zhang, G. Liu, Z. Wang, M. Chappell, Y. Liu, D. Zheng, A. Leibbrandt, T. Wada, A. S. Slutsky, D. Liu, C. Qin, C. Jiang, and J. M. Penninger. 2005. ‘A crucial role of angiotensin converting enzyme 2 (ACE2) in SARS coronavirus-induced lung injury’, Nat Med, 11: 875-9.
Lim, B. K., J. H. Choi, J. H. Nam, C. O. Gil, J. O. Shin, S. H. Yun, D. K. Kim, and E. S. Jeon. 2006. ‘Virus receptor trap neutralizes coxsackievirus in experimental murine viral myocarditis’, Cardiovasc Res, 71: 517-26.
Liu, Z., X. Xiao, X. Wei, J. Li, J. Yang, H. Tan, J. Zhu, Q. Zhang, J. Wu, and L. Liu. 2020. ‘Composition and divergence of coronavirus spike proteins and host ACE2 receptors predict potential intermediate hosts of SARS-CoV-2’, J Med Virol.
Lu, G., Y. Hu, Q. Wang, J. Qi, F. Gao, Y. Li, Y. Zhang, W. Zhang, Y. Yuan, J. Bao, B. Zhang, Y. Shi, J. Yan, and G. F. Gao. 2013. ‘Molecular basis of binding between novel human coronavirus MERS-CoV and its receptor CD26’, Nature, 500: 227-31.
Maclean, J., M. Koekemoer, A. J. Olivier, D. Stewart, Hitzeroth, II, T. Rademacher, R. Fischer, A. L. Williamson, and E. P. Rybicki. 2007. ‘Optimization of human papillomavirus type 16 (HPV-16) L1 expression in plants: comparison of the suitability of different HPV-16 L1 gene variants and different cell-compartment localization’, J Gen Virol, 88: 1460-9.
Martin, S., J. M. Casasnovas, D. E. Staunton, and T. A. Springer. 1993. ‘Efficient neutralization and disruption of rhinovirus by chimeric ICAM-1/immunoglobulin molecules’, J Virol, 67: 3561-8.
Neuman, B. W., B. D. Adair, C. Yoshioka, J. D. Quispe, G. Orca, P. Kuhn, R. A. Milligan, M. Yeager, and M. J. Buchmeier. 2006. ‘Supramolecular architecture of severe acute respiratory syndrome coronavirus revealed by electron cryomicroscopy’, J Virol, 80: 7918-28.
Petruccelli, S., M. S. Otegui, F. Lareu, O. Tran Dinh, A. C. Fitchette, A. Circosta, M. Rumbo, M. Bardor, R. Carcamo, V. Gomord, and R. N. Beachy. 2006. ‘A KDEL-tagged monoclonal antibody is efficiently retained in the endoplasmic reticulum in leaves, but is both partially secreted and sorted to protein storage vacuoles in seeds’, Plant Biotechnol J, 4: 511-27.
Raj, V. S., H. Mou, S. L. Smits, D. H. Dekkers, M. A. Muller, R. Dijkman, D. Muth, J. A. Demmers, A. Zaki, R. A. Fouchier, V. Thiel, C. Drosten, P. J. Rottier, A. D. Osterhaus, B. J. Bosch, and B. L. Haagmans. 2013. ‘Dipeptidyl peptidase 4 is a functional receptor for the emerging human coronavirus-EMC’, Nature, 495: 251-4.
Rapaka, R. R., E. S. Goetzman, M. Zheng, J. Vockley, L. McKinley, J. K. Kolls, and C. Steele. 2007. ‘Enhanced defense against Pneumocystis carinii mediated by a novel dectin-1 receptor Fc fusion protein’, J Immunol, 178: 3702-12.
Rath, T., K. Baker, J. A. Dumont, R. T. Peters, H. Jiang, S. W. Qiao, W. I. Lencer, G. F. Pierce, and R. S. Blumberg. 2013. ‘Fc-fusion proteins and FcRn: structural insights for longer-lasting and more effective therapeutics’, Crit Rev Biotechnol.
Reinhart, D., R. Weik, and R. Kunert. 2012. ‘Recombinant IgA production: single step affinity purification using camelid ligands and product characterization’, J Immunol Methods, 378: 95-101.
Rockx, B., E. Donaldson, M. Frieman, T. Sheahan, D. Corti, A. Lanzavecchia, and R. S. Baric. 2010. ‘Escape from human monoclonal antibody neutralization affects in vitro and in vivo fitness of severe acute respiratory syndrome coronavirus’, J Infect Dis, 201: 946-55.
Silberstein, E., L. Xing, W. van de Beek, J. Lu, H. Cheng, and G. G. Kaplan. 2003. ‘Alteration of hepatitis A virus (HAV) particles by a soluble form of HAV cellular receptor 1 containing the immunoglobin- and mucin-like regions’, J Virol, 77: 8765-74.
Spiekermann, G. M., P. W. Finn, E. S. Ward, J. Dumont, B. L. Dickinson, R. S. Blumberg, and W. I. Lencer. 2002. ‘Receptor-mediated immunoglobulin G transport across mucosal barriers in adult life: functional expression of FcRn in the mammalian lung’, J Exp Med, 196: 303-10.
Strasser, R., A. Castilho, J. Stadlmann, R. Kunert, H. Quendler, P. Gattinger, T. Rademacher, F. Altmann, L. Mach, and H. Steinkellner. 2009. ‘Improved virus neutralization by plant-produced anti-HIV antibodies with a homogeneous beta1,4-galactosylated N-glycan profile’, J Biol Chem, 284: 20479-85.
Strasser, R., J. Stadlmann, M. Schahs, G. Stiegler, H. Quendler, L. Mach, J. Glossl, K. Weterings, M. Pabst, and H. Steinkellner. 2008. ‘Generation of glyco-engineered Nicotiana benthamiana for the production of monoclonal antibodies with a homogeneous human-like N-glycan structure’, Plant Biotechnol J, 6: 392-402.
Sui, J., M. Deming, B. Rockx, R. C. Liddington, Q. K. Zhu, R. S. Baric, and W. A. Marasco. 2014. ‘Effects of human anti-spike protein receptor binding domain antibodies on severe acute respiratory syndrome coronavirus neutralization escape and fitness’, J Virol, 88: 13769-80.
Tao, X., T. E. Hill, C. Morimoto, C. J. Peters, T. G. Ksiazek, and C. T. Tseng. 2013. ‘Bilateral entry and release of Middle East respiratory syndrome coronavirus induces profound apoptosis of human bronchial epithelial cells’, J Virol, 87: 9953-8.
Tseng, C. T., C. Huang, P. Newman, N. Wang, K. Narayanan, D. M. Watts, S. Makino, M. M. Packard, S. R. Zaki, T. S. Chan, and C. J. Peters. 2007. ‘Severe acute respiratory syndrome coronavirus infection of mice transgenic for the human Angiotensin-converting enzyme 2 virus receptor’, J Virol, 81: 1162-73.
Vaquero, C., M. Sack, J. Chandler, J. Drossard, F. Schuster, M. Monecke, S. Schillberg, and R. Fischer. 1999. ‘Transient expression of a tumor-specific single-chain fragment and a chimeric antibody in tobacco leaves’, Proceedings of the National Acadamy of Sciences USA, 96: 11128-33.
Vickers, C., P. Hales, V. Kaushik, L. Dick, J. Gavin, J. Tang, K. Godbout, T. Parsons, E. Baronas, F. Hsieh, S. Acton, M. Patane, A. Nichols, and P. Tummino. 2002. ‘Hydrolysis of biological peptides by human angiotensin-converting enzyme-related carboxypeptidase’, J Biol Chem, 277: 14838-43.
Voinnet, O., S. Rivas, P. Mestre, and D. Baulcombe. 2003. ‘An enhanced transient expression system in plants based on suppression of gene silencing by the p19 protein of tomato bushy stunt virus’, The Plant Journal, 33: 949-56.
Wan, Y., J. Shang, R. Graham, R. S. Baric, and F. Li. 2020. ‘Receptor recognition by novel coronavirus from Wuhan: An analysis based on decade-long structural studies of SARS’, J Virol.
Wang, W., S. M. McKinnie, M. Farhan, M. Paul, T. McDonald, B. McLean, C. Llorens-Cortes, S. Hazra, A. G. Murray, J. C. Vederas, and G. Y. Oudit. 2016. ‘Angiotensin-Converting Enzyme 2 Metabolizes and Partially Inactivates Pyr-Apelin-13 and Apelin-17: Physiological Effects in the Cardiovascular System’, Hypertension, 68: 365-77.
Ward, R. H., D. J. Capon, C. M. Jett, K. K. Murthy, J. Mordenti, C. Lucas, S. W. Frie, A. M. Prince, J. D. Green, and J. W. Eichberg. 1991. ‘Prevention of HIV-1 IIIB infection in chimpanzees by CD4 immunoadhesin’, Nature, 352: 434-6.
Zhou, P., X. L. Yang, X. G. Wang, B. Hu, L. Zhang, W. Zhang, H. R. Si, Y. Zhu, B. Li, C. L. Huang, H. D. Chen, J. Chen, Y. Luo, H. Guo, R. D. Jiang, M. Q. Liu, Y. Chen, X. R. Shen, X. Wang, X. S. Zheng, K. Zhao, Q. J. Chen, F. Deng, L. L. Liu, B. Yan, F. X. Zhan, Y. Y. Wang, G. F. Xiao, and Z. L. Shi. 2020. ‘A pneumonia outbreak associated with a new coronavirus of probable bat origin’, Nature.

1. A modified human ACE2 protein comprising at least amino acids 19-614 of the human ACE2 protein sequence of SEQ ID NO: 1 with at least one consensus contact sequence residue altered relative to SEQ ID NO: 1, wherein affinity of the modified human ACE2 protein for the S1 spike protein of SARS-CoV-2 is increased relative to affinity of the human ACE2 protein of SEQ ID NO: 1 for the S1 spike protein of SARS CoV-2.

2. The ACE2 protein of claim 1, wherein said at least one altered residue is selected from amino acid residues 30-42; optionally, wherein said at least one altered residue is selected from amino acid residues 30, 31, 34, 38, 40, and 41.

3. (canceled)

4. The ACE2 protein of claim 2, wherein said at least one altered residue comprises an amino acid change selected from D30E, D30S, K31Q, K31E, H34S, H34V, D38E, F40S, Q42A, and combinations thereof; optionally, wherein the at least one residue altered comprises the two amino acid changes D38E and F40S.

5. The ACE2 protein of claim 1, wherein said at least one altered residue is selected from amino acid residues 81-84; optionally, wherein said at least one altered residue is selected from amino acids 81 and 82.

6. (canceled)

7. The ACE2 protein of claim 5, wherein said at least one altered residue comprises an amino acid change selected from Q81K, M82N, M82K, M82T, and combinations thereof; optionally wherein the at least one altered residue comprises at least the two amino acid changes Q81K and M82N.

8. The ACE2 protein of claim 1, wherein said at least one altered residue is selected from amino acid residues 353-357; optionally, wherein said at least one altered residue comprises an amino acid change selected from G354H or G354K.

9. (canceled)

10. The ACE2 protein of claim 1, wherein said at least one residue altered is selected from amino acid residues 327-329; optionally, wherein said at least one residue altered comprises an amino acid change selected from E329N or E329K.

11. (canceled)

12. The ACE2 protein of claim 1, wherein at least one N-glycosylation site residue is changed from an N to an amino acid residue that does not glycosylate; optionally, wherein the at least one N-glycosylation site is changed from an N to S.

13. The ACE2 protein of claim 12, wherein the N-glycosylation site residue at position 546 has been changed from N to S.

14. The ACE2 protein of claim 1, wherein an amino acid residue adjacent to an N-glycosylation site residue is changed to an amino acid residue that prevents glycosylation at the adjacent N-glycosylation site; optionally, wherein the amino acid residue at position 547 adjacent to the N-glycosylation site at position 546 is changed from S to P.

15. (canceled)

16. The ACE2 protein of claim 12, further comprising an amino acid change selected from H34S and H34V.

17. The ACE2 protein of claim 1 further comprising a fusion with a human immunoglobulin Fc region.

18. The ACE2 protein of claim 17, wherein said immunoglobulin is selected from the group consisting of IgG1, IgG2, IgG3, IgG4, IgA1, IgA2, IgM, IgD, and IgE; optionally, wherein the human immunoglobulin Fc region comprises an amino acid sequence of any one of SEQ ID NOs: 18-22.

19. (canceled)

20. (canceled)

21. The ACE2 protein of claim 17, wherein said Fc region further comprises a KDEL sequence at the carboxy terminus thereof.

22. (canceled)

23. (canceled)

24. (canceled)

25. The ACE2 protein of claim 1, wherein the protein has typical mammalian glycosylation.

26. The ACE2 protein of claim 1, wherein N-glycans of said protein lack detectable β1,2-xylose and α1,3-fucose residues; optionally, wherein the protein comprises a terminal β1,4-Gal residue at an N-glycan.

27. (canceled)

28. The ACE2 protein of claim 26, wherein the protein is produced in a DXT/FT plant; optionally, wherein the plant is N. benthamiana.

29. (canceled)

30. (canceled)

31. (canceled)

32. (canceled)

33. A nucleic acid sequence encoding an ACE2 protein of claim 1.

34. (canceled)

35. A chimeric SARS-CoV-2 S1 spike protein receptor comprising: an immunoglobulin complex, wherein the immunoglobulin complex comprises at least a portion of an immunoglobulin heavy chain, and a modified ACE2 protein linked to the immunoglobulin heavy chain, wherein the modified ACE2 protein comprises at least amino acids 19-614 of the human ACE2 protein sequence of SEQ ID NO: 1 with at least one consensus contact sequence residue altered relative to SEQ ID NO: 1 to increase affinity for the SARS-Cov-2 S1 spike protein.

36. (canceled)

37. (canceled)

38. (canceled)

39. (canceled)

40. (canceled)

41. (canceled)

42. A composition comprising the chimeric SARS-CoV-2 S1 spike protein receptor of claim 35 and plant material.

43. (canceled)

44. A method for reducing binding of SARS-CoV-2 to a host cell, the method comprising: contacting the SARS-CoV-2 with the chimeric SARS-CoV-2 S1 spike protein receptor of claim 35, wherein the chimeric receptor binds to the SARS-CoV-2 Receptor Binding Domain (RBD) thereby reducing the binding of SARS-CoV-2 to the host cell.

45. (canceled)

46. A chimeric SARS-CoV-2 S1 spike protein receptor of claim 35 for use in preventing or treating SARS-CoV-1 infection, or the effects thereof.

47. An expression vector encoding a chimeric SARS-CoV-2 S1 spike protein receptor of claim 35.

48. (canceled)

49. (canceled)

50. A pharmaceutical composition comprising a chimeric SARS-CoV-2 S1 spike protein receptor as claimed in claim 35 and a pharmaceutically acceptable carrier.

51. (canceled)

52. (canceled)

53. A pharmaceutical composition comprising a modified human ACE2 protein claim 1 and a pharmaceutically acceptable carrier.

54. A method for reducing binding of SARS-CoV-2 to a cell, the method comprising: contacting the SARS-CoV-2 with the modified human ACE2 protein of claim 1, wherein the modified human ACE2 protein binds to the SARS-CoV-2 Receptor Binding Domain (RBD), thereby reducing the binding of SARS-CoV-2 to the cell.

55. (canceled)

56. (canceled)

57. The modified human ACE2 protein of claim 1 for use in treating or preventing a SARS-CoV-2 infection, or the effects thereof.


SEQUENCE LISTING (127 sequences)

SEQ ID NO: 1 - protein, 805 aa, Homo sapiens [sequence omitted]

SEQ ID NO: 2 - protein, 596 aa, Homo sapiens
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 3 - DNA, 2166 bp, Homo sapiens
tccaccatcgaggaacaggctaagaccttcctcgacaagttcaaccacgaggccgaggacctcttctaccagtcctccctcgcctcctggaactacaacaccaacatcaccgaggagaacgtgcagaacatgaacaacgccggcgacaagtggtccgccttcctcaaggagcagtccaccctcgcccagatgtacccactccaggagatccagaacctcaccgtgaagttgcagctccaggccctccagcagaacggatcctccgtgctctccgaggacaagtccaagaggctcaacaccatcctcaacaccatgtccaccatctactccaccggaaaggtgtgcaaccccgacaacccccaggagtgcctcttgctcgagcccggcctcaacgagatcatggccaactccctcgactacaacgagaggctctgggcctgggagtcctggcgttccgaggtcggaaagcagctcaggcccctctacgaggagtacgtggtgctcaagaacgagatggccagggccaaccactacgaggactacggtgactattggaggggcgactacgaggtgaacggtgtggacggatacgactactccaggggtcagctcatcgaggacgtcgagcacaccttcgaggagatcaagcccctctacgagcacctccacgcctacgttagggccaagctgatgaacgcctacccctcctacatttcccccatcggatgtttgcccgcccacctcctcggagatatgtggggaaggttctggaccaacctctactccctcaccgtgcccttcggccagaagcccaacatcgacgtgaccgacgctatggtggaccaagcctgggacgcccagaggatcttcaaggaggccgagaagttcttcgtgtccgtgggactccccaacatgacccaaggattctgggagaactccatgctcaccgatcccggcaacgtgcagaaggccgtctgccaccccaccgcctgggacctcggaaagggcgacttcaggatcctcatgtgcaccaaggtgactatggacgacttcctcaccgcccaccacgagatgggacacatccagtacgacatggcctacgccgcccagcccttcctcctcaggaacggtgccaacgagggattccacgaggccgtgggagagatcatgtccctctccgccgccactcccaagcacctcaagtccatcggactcctctcacccgacttccaggaggacaacgagaccgagatcaacttcctgctcaagcaggccctcaccatcgtgggaaccctccccttcacctacatgctcgagaagtggcgttggatggtgttcaagggcgagatccccaaggaccagtggatgaagaagtggtgggagatgaagcgtgagatcgtcggagttgtcgagcccgtcccccacgacgagacctactgcgaccccgcctccctcttccacgtctccaacgactactccttcatcaggtactacaccaggaccctgtaccagttccagtttcaggaggcactctgccaggccgccaagcacgagggacccctccacaagtgcgacatctccaactccaccgaggccggccagaaactcttcaacatgctcaggctcggaaagtccgagccctggaccctcgccctcgagaacgtcgtgggcgccaagaacatgaacgtgaggccactcctcaactacttcgagcccctcttcacctggctcaaggaccagaacaagaactccttcgtcggctggtccaccgactggtccccttatgcggaccagtccatcaaggtgaggatctccctcaagtccgccctcggcgacaaggcctacgagtggaacgacaacgagatgtacctcttcaggtcctccgtggcctatgctatgaggcagtacttcctgaaggtgaagaaccagatgatcctcttcggcgaggaggacgtgagggtcgccaatctcaagcccaggatctccttcaacttcttcgtgaccgcccccaagaacgtgtccgacatcatccccaggaccgaggtcgagaaggccatcaggatgtctaggtccaggatcaacgacgccttcaggctcaacgacaactccctcgagttcctcggaatccagcccaccctcggaccacccaaccagccccccgtctcg

SEQ ID NO: 4 - protein, 828 aa, Artificial
  Synthetic polypeptide
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 828 aa, Artificial
  Synthetic polypeptide
STIEEQAKTFLDKFNSEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 830 aa, Artificial
  Synthetic polypeptide
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAPVPSTPPTPSPSTPPTPSPSCCHPRLSLHRPALEDLLLGSEAQLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGK

SEQ ID NO: 7 - protein, 816 aa, Artificial
  Synthetic polypeptide
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAPVPPPPPCCHPRLSLHRPALEDLLLGSEAQLTCTLTGLRDASGATFTWTPSSGKSAVQGPPERDLCGCYSVSSVLPGCAQPWNHGETFTCTAAHPELKTPLTAQITKSGNTFRPEVHLLPPPSEELALNELVTLTCLARGFSPKDVLVRWLQGSQELPRKYLTWASRQEPSQGTTTFAVTSILRVAAEDWKKGDTFSCMVGHEALPLAFTQKTIDRLAGK

SEQ ID NO: 8 - protein, 828 aa, Artificial
  Synthetic polypeptide
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 9 - protein, 830 aa, Artificial
  Synthetic polypeptide
PVPSTPPTPSPSTPPTPSPSCCHPRLSLHRPALEDLLLGSEAQLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 10 - protein, 817 aa, Artificial
  Synthetic polypeptide
PVPPPPPCCHPRLSLHRPALEDLLLGSEAQLTCTLTGLRDASGATFTWTPSSGKSAVQGPPERDLCGCYSVSSVLPGCAQPWNHGETFTCTAAHPELKTPLTAQITKSGNTFRPEVHLLPPPSEELALNELVTLTCLARGFSPKDVLVRWLQGSQELPRKYLTWASRQEPSQGTTTFAVTSILRVAAEDWKKGDTFSCMVGHEALPLAFTQKTIDRLAGKSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 11 - protein, 828 aa, Artificial
  Synthetic polypeptide
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAEPKSCDKTHTCPPCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNHFTQKSLSLSPGK

SEQ ID NO: 12 - protein, 828 aa, Artificial
  Synthetic polypeptide
EPKSCDKTHTCPPCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNHFTQKSLSLSPGKSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 13 - protein, 828 aa, Artificial
  Synthetic polypeptide
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAEPKSADKTHTCPPCPAPELLGGPSVFLFPPKPKDTLMISRTPEVTCVVVDVSHEAPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 828 aa, Artificial
  Synthetic polypeptide
EPKSADKTHTCPPCPAPELLGGPSVFLFPPKPKDTLMISRTPEVTCVVVDVSHEAPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYEYGDYWRGDVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 15 - protein, 825 aa, Artificial
  Synthetic polypeptide
STIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 825 aa, Artificial
  Synthetic polypeptide
STIEEQAKTFLDKFNSEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 825 aa, Artificial
  Synthetic polypeptide
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYEYGDYWRGDVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYA

SEQ ID NO: 18 - protein, 232 aa, Homo sapiens
EPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 229 aa, Homo sapiens
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 232 aa, Homo sapiens
EPKSCDKTHTCPPCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNHFTQKSLSLSPGK

SEQ ID NO: 21 - protein, 234 aa, Homo sapiens
PVPSTPPTPSPSTPPTPSPSCCHPRLSLHRPALEDLLLGSEAQLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGK

SEQ ID NO: 22 - protein, 220 aa, Homo sapiens
PVPPPPPCCHPRLSLHRPALEDLLLGSEAQLTCTLTGLRDASGATFTWTPSSGKSAVQGPPERDLCGCYSVSSVLPGCAQPWNHGETFTCTAAHPELKTPLTAQITKSGNTFRPEVHLLPPPSEELALNELVTLTCLARGFSPKDVLVRWLQGSQELPRKYLTWASRQEPSQGTTTFAVTSILRVAAEDWKKGDTFSCMVGHEALPLAFTQKTIDRLAGK

SEQ ID NO: 23 - protein, 5 aa, Artificial
  Synthetic polypeptide
GGGGS

SEQ ID NO: 24 - protein, 10 aa, Artificial
  Synthetic polypeptide
GGGGSGGGGS

SEQ ID NO: 25 - protein, 15 aa, Artificial
  Synthetic polypeptide
GGGGSGGGGSGGGGS

SEQ ID NO: 26 - protein, 4 aa, Artificial
  Synthetic polypeptide
GGGS

SEQ ID NO: 27 - protein, 8 aa, Artificial
  Synthetic polypeptide
GGGSGGGS

SEQ ID NO: 28 - protein, 12 aa, Artificial
  Synthetic polypeptide
GGGSGGGSGGGS

SEQ ID NO: 29 - protein, 57 aa, artificial
  Artificial polypeptide
GGGGSGGGGSGGGGSGGGGSGGGGSASGGGGSGGGGSGGGGSGGGGSGGGGSGGGGS

SEQ ID NO: 30 - protein, 35 aa, artificial
  Artificial polypeptide
AGGGSGGGGSGGGGSGGGGSGGGGSGGGGSGGGGS

SEQ ID NO: 31 - protein, 40 aa, artificial
  Artificial polypeptide
AGGGSGGGGSGGGGSGGGGSGGGGSGGGGSGGGGSGGGGS

SEQ ID NO: 32 - protein, 45 aa, artificial
  Artificial polypeptide
AGGGSGGGGSGGGGSGGGGSGGGGSGGGGSGGGGSGGGGSGGGGS

SEQ ID NO: 33 - protein, 14 aa, artificial
  Artificial polypeptide
EGKSSGSGSESKEF

SEQ ID NO: 34 - protein, 72 aa, artificial
  Artificial polypeptide
AGSGGSGGSGGSPVPSTPPTNSSSTPPTPSPSPVPSTPPTNSSSTPPTPSPSPVPSTPPTNSSSTPPTPSPS

SEQ ID NO: 35 - protein, 72 aa, artificial
  Artificial polypeptide
AGSGGSGGSGGSPVPSTPPTPSPSTPPTPSPSPVPSTPPTNSSSTPPTPSPSPVPSTPPTPSPSTPPTPSPS

SEQ ID NO: 36 - protein, 64 aa, artificial
  Artificial polypeptide
AGSGGSGGSGGSPVPSTPPTPSPSTPPTPSPSGGSGNSSGSGGSPVPSTPPTPSPSTPPTPSPS

SEQ ID NO: 37 - protein, 72 aa, artificial
  Artificial polypeptide
AGSGGSGGSGGSPVPSTPPTPSPSTPPTPSPSPVPSTPPTPSPSTPPTPSPSPVPSTPPTPSPSTPPTPSPS

SEQ ID NO: 38 - protein, 150 aa, artificial
  Artificial polypeptide
AGSGGSGGSGGSPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDPVPSTPPTPSPSTPPTPSPS

SEQ ID NO: 39 - protein, 142 aa, artificial
  Artificial polypeptide
AGSGGSGGSGGSPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSGGS

SEQ ID NO: 40 - protein, 240 aa, artificial
  Artificial polypeptide
AGPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSG

SEQ ID NO: 41 - protein, 260 aa, artificial
  Artificial polypeptide
AGSGGSGGSGGSPVPSTPPTPSPSTPPTPSPSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDGGSGGSGGSGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDPVPSTPPTPSPSTPPTPSPS

SEQ ID NO: 42 - protein, 119 aa, artificial
  Artificial polypeptide
AGSGGSGGSGGSPVPSTPPTPSPSTPPTPSPSQIFVKTLTGKTITLEVEPSDTIENVKAKIQDKEGIPPDQQRLIFAGKQLEDGRTLSDYNIQKESTLHLVLRLRGGGGSGGSGGSGGS

SEQ ID NO: 43 - protein, 324 aa, artificial
  Artificial polypeptide
AGSGGSGGSGGSPVPSTPPTPSPSTPPTPSPSDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEASPVPSTPPTPSPSTPPTPS

SEQ ID NO: 44 - protein, 604 aa, artificial
  Artificial polypeptide
AGSGGSGGSGGSGGSGGSGGSGGSDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEASGGSGGSGGSGGSDGRYSLTYIYTGLSKHVEDVPAFQALGSLNDLQFFRYNSKDRKSQPMGLWRQVEGMEDWKQDSQLQKAREDIFMETLKDIVEYYNDSNGSHVLQGRFGCEIENNRSSGAFWKYYYDGKDYIEFNKEIPAWVPFDPAAQITKQKWEAEPVYVQRAKAYLEEECPATLRKYLKYSKNILDRQDPPSVVVTSHQAPGEKKKLKCLAYDFYPGKIDVHWTRAGEVQEPELRGDVLHNGNGTYQSWVVVAVPPQDTAPYSCHVQHSSLAQPLVVPWEASPVPSTPPTPSPSTPPTPSPS

SEQ ID NO: 45 - protein, 44 aa, artificial
  Artificial polypeptide
AGSGNSSGSGGSGGSGNSSGSGGSPVPSTPPTPSPSTPPTPSPS

SEQ ID NO: 46 - protein, 139 aa, artificial
  Artificial polypeptide
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGG

SEQ ID NO: 47 - protein, 243 aa, artificial
  Artificial polypeptide
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSRSAGGGGSGGGGSGGGG

SEQ ID NO: 48 - protein, 347 aa, artificial
  Artificial polypeptide
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQRSAGGGGSGGGGSGGGG

SEQ ID NO: 49 - protein, 449 aa, artificial
  Artificial polypeptide
KLSGGGGSGGGGSGGGGSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAEAWYNLGNAYYKQGDYQKAIEYYQKALELDPNNAEAWYNLGNAYYKQGDYQKAIEYYQKALELDPNNAEAWYNLGNAYYKQGDYQKAIEDYQKALELDPNNLQAEAWKNLGNAYYKQGDYQKAIEYYQKALELDPNNASAWYNLGNAYYKQGDYQKAIEYYQKALELDPNNAKAWYRRGNAYYKQGDYQKAIEDYQKALELDPNNRSAGGGGSGGGGSGGGG

SEQ ID NO: 50 - DNA, 39 bp, artificial
  Artificial polynucleotide
caggctaagaccttcctcgagaagttcaaccacgaggcc

SEQ ID NO: 51 - DNA, 39 bp, artificial
  Artificial polynucleotide
ggcctcgtggttgaacttctcgaggaaggtcttagcctg

SEQ ID NO: 52 - DNA, 39 bp, artificial
  Artificial polynucleotide
caggctaagaccttcctctccaagttcaaccacgaggcc

SEQ ID NO: 53 - DNA, 39 bp, artificial
  Artificial polynucleotide
ggcctcgtggttgaacttggagaggaaggtcttagcctg

SEQ ID NO: 54 - DNA, 39 bp, artificial
  Artificial polynucleotide
gctaagaccttcctcgaccagttcaaccacgaggccgag

SEQ ID NO: 55 - DNA, 39 bp, artificial
  Artificial polynucleotide
ctcggcctcgtggttgaactggtcgaggaaggtcttagc

SEQ ID NO: 56 - DNA, 39 bp, artificial
  Artificial polynucleotide
gctaagaccttcctcgacgagttcaaccacgaggccgag

SEQ ID NO: 57 - DNA, 39 bp, artificial
  Artificial polynucleotide
ctcggcctcgtggttgaactcgtcgaggaaggtcttagc

SEQ ID NO: 58 - DNA, 39 bp, artificial
  Artificial polynucleotide
ttcctcgacaagttcaactccgaggccgaggacctcttc

SEQ ID NO: 59 - DNA, 39 bp, artificial
  Artificial polynucleotide
gaagaggtcctcggcctcggagttgaacttgtcgaggaa

SEQ ID NO: 60 - DNA, 39 bp, artificial
  Artificial polynucleotide
ttcctcgacaagttcaacgtggaggccgaggacctcttc

SEQ ID NO: 61 - DNA, 39 bp, artificial
  Artificial polynucleotide
gaagaggtcctcggcctccacgttgaacttgtcgaggaa

SEQ ID NO: 62 - DNA, 39 bp, artificial
  Artificial polynucleotide
ttcaaccacgaggccgaggagctcttctaccagtcctcc

SEQ ID NO: 63 - DNA, 39 bp, artificial
  Artificial polynucleotide
ggaggactggtagaagagctcctcggcctcgtggttgaa

SEQ ID NO: 64 - DNA, 39 bp, artificial
  Artificial polynucleotide
gccgaggacctcttctacgcctcctccctcgcctcctgg

SEQ ID NO: 65 - DNA, 39 bp, artificial
  Artificial polynucleotide
ccaggaggcgagggaggaggcgtagaagaggtcctcggc

SEQ ID NO: 66 - DNA, 39 bp, artificial
  Artificial polynucleotide
gcagtccaccctcgcccagaactacccactccaggagat

SEQ ID NO: 67 - DNA, 39 bp, artificial
  Artificial polynucleotide
atctcctggagtgggtagttctgggcgagggtggactgc

SEQ ID NO: 68 - DNA, 39 bp, artificial
  Artificial polynucleotide
cagtccaccctcgcccagaagtacccactccaggagatc

SEQ ID NO: 69 - DNA, 39 bp, artificial
  Artificial polynucleotide
gatctcctggagtgggtacttctgggcgagggtggactg

SEQ ID NO: 70 - DNA, 39 bp, artificial
  Artificial polynucleotide
gcctgggacctcggaaagcacgacttcaggatcctcatg

SEQ ID NO: 71 - DNA, 39 bp, artificial
  Artificial polynucleotide
catgaggatcctgaagtcgtgctttccgaggtcccaggc

SEQ ID NO: 72 - DNA, 39 bp, artificial
  Artificial polynucleotide
gcctgggacctcggaaagaaggacttcaggatcctcatg

SEQ ID NO: 73 - DNA, 39 bp, artificial
  Artificial polynucleotide
catgaggatcctgaagtccttctttccgaggtcccaggc

SEQ ID NO: 74 - DNA, 42 bp, artificial
  Artificial polynucleotide
caaccacgaggccgaggagctctcctaccagtcctccctcgc

SEQ ID NO: 75 - DNA, 42 bp, artificial
  Artificial polynucleotide
gcgagggaggactggtaggagagctcctcggcctcgtggttg

SEQ ID NO: 76 - DNA, 41 bp, artificial
  Artificial polynucleotide
gagcagtccaccctcgccaagaactacccactccaggagat

SEQ ID NO: 77 - DNA, 41 bp, artificial
  Artificial polynucleotide
atctcctggagtgggtagttcttggcgagggtggactgctc

SEQ ID NO: 78 - DNA, 39 bp, artificial
  Artificial polynucleotide
cagtccaccctcgcccagacctacccactccaggagatc

SEQ ID NO: 79 - DNA, 39 bp, artificial
  Artificial polynucleotide
gatctcctggagtgggtaggtctgggcgagggtggactg

SEQ ID NO: 80 - DNA, 39 bp, artificial
  Artificial polynucleotide
atgacccaaggattctggaacaactccatgctcaccgat

SEQ ID NO: 81 - DNA, 39 bp, artificial
  Artificial polynucleotide
atcggtgagcatggagttgttccagaatccttgggtcat

SEQ ID NO: 82 - DNA, 39 bp, artificial
  Artificial polynucleotide
ctgcaggtgttcactccccgaccatcgaggaacaggcta

SEQ ID NO: 83 - DNA, 39 bp, artificial
  Artificial polynucleotide
tagcctgttcctcgatggtcggggagtgaacacctgcag

SEQ ID NO: 84 - DNA, 39 bp, artificial
  Artificial polynucleotide
ctccaggccctccagcagtcgggatcctccgtgctctcc

SEQ ID NO: 85 - DNA, 39 bp, artificial
  Artificial polynucleotide
ggagagcacggaggatcccgactgctggagggcctggag

SEQ ID NO: 86 - DNA, 39 bp, artificial
  Artificial polynucleotide
atcccggcaacgtgcagcgcgccgtctgccaccccaccg

SEQ ID NO: 87 - DNA, 39 bp, artificial
  Artificial polynucleotide
cggtggggtggcagacggcgcgctgcacgttgccgggat

SEQ ID NO: 88 - DNA, 39 bp, artificial
  Artificial polynucleotide
tggtgttcaagggcgaggtccccaaggaccagtggatga

SEQ ID NO: 89 - DNA, 39 bp, artificial
  Artificial polynucleotide
tcatccactggtccttggggacctcgcccttgaacacca

SEQ ID NO: 90 - DNA, 39 bp, artificial
  Artificial polynucleotide
acaagtgcgacatctccagctccaccgaggccggccaga

SEQ ID NO: 91 - DNA, 39 bp, artificial
  Artificial polynucleotide
tctggccggcctcggtggagctggagatgtcgcacttgt

SEQ ID NO: 92 - DNA, 39 bp, artificial
  Artificial polynucleotide
agtgcgacatctccaacccgaccgaggccggccagaaac

SEQ ID NO: 93 - DNA, 39 bp, artificial
  Artificial polynucleotide
gtttctggccggcctcggtcgggttggagatgtcgcact

SEQ ID NO: 94 - DNA, 39 bp, artificial
  Artificial polynucleotide
gcgacatctccaactccgcggaggccggccagaaactct

SEQ ID NO: 95 - DNA, 39 bp, artificial
  Artificial polynucleotide
agagtttctggccggcctccgcggagttggagatgtcgc

SEQ ID NO: 96 - DNA, 39 bp, artificial; identical to SEQ ID NO: 86
  Artificial polynucleotide

SEQ ID NO: 97 - DNA, 39 bp, artificial; identical to SEQ ID NO: 86
  Artificial polynucleotide

SEQ ID NO: 98 - DNA, 39 bp, artificial; identical to SEQ ID NO: 60
  Artificial polynucleotide

SEQ ID NO: 99 - DNA, 39 bp, artificial; identical to SEQ ID NO: 61
  Artificial polynucleotide

SEQ ID NO: 100 - DNA, 39 bp, artificial; identical to SEQ ID NO: 82
  Artificial polynucleotide

SEQ ID NO: 101 - DNA, 39 bp, artificial; identical to SEQ ID NO: 83
  Artificial polynucleotide

SEQ ID NO: 102 - DNA, 39 bp, artificial; identical to SEQ ID NO: 84
  Artificial polynucleotide

SEQ ID NO: 103 - DNA, 39 bp, artificial; identical to SEQ ID NO: 85
  Artificial polynucleotide

SEQ ID NO: 104 - DNA, 39 bp, artificial; identical to SEQ ID NO: 86
  Artificial polynucleotide

SEQ ID NO: 105 - DNA, 39 bp, artificial; identical to SEQ ID NO: 87
  Artificial polynucleotide

SEQ ID NO: 106 - DNA, 39 bp, artificial; identical to SEQ ID NO: 88
  Artificial polynucleotide

SEQ ID NO: 107 - DNA, 39 bp, artificial; identical to SEQ ID NO: 89
  Artificial polynucleotide

SEQ ID NO: 108 - DNA, 39 bp, artificial; identical to SEQ ID NO: 90
  Artificial polynucleotide

SEQ ID NO: 109 - DNA, 39 bp, artificial; identical to SEQ ID NO: 91
  Artificial polynucleotide

SEQ ID NO: 110 - DNA, 39 bp, artificial; identical to SEQ ID NO: 92
  Artificial polynucleotide

SEQ ID NO: 111 - DNA, 39 bp, artificial; identical to SEQ ID NO: 93
  Artificial polynucleotide

SEQ ID NO: 112 - DNA, 39 bp, artificial; identical to SEQ ID NO: 94
  Artificial polynucleotide

SEQ ID NO: 113 - DNA, 39 bp, artificial; identical to SEQ ID NO: 95
  Artificial polynucleotide

SEQ ID NO: 114 - DNA, 43 bp, artificial
  Artificial polynucleotide
gaaactgcaggtgttcactccccgaccatcgaggaacaggcta

SEQ ID NO: 115 - DNA, 39 bp, artificial; identical to SEQ ID NO: 58
  Artificial polynucleotide

SEQ ID NO: 116 - DNA, 39 bp, artificial; identical to SEQ ID NO: 59
  Artificial polynucleotide

SEQ ID NO: 117 - DNA, 39 bp, artificial; identical to SEQ ID NO: 60
  Artificial polynucleotide

SEQ ID NO: 118 - DNA, 39 bp, artificial; identical to SEQ ID NO: 61
  Artificial polynucleotide

SEQ ID NO: 119 - DNA, 42 bp, artificial
  Artificial polynucleotide
tcctcggagatatgtggggaaggttctggaccaacctctact

SEQ ID NO: 120 - DNA, 42 bp, artificial
  Artificial polynucleotide
agtagaggttggtccagaaccttccccacatatctccgagga

SEQ ID NO: 121 - DNA, 20 bp, artificial
  Artificial polynucleotide
tggagtggagctggatcttc

SEQ ID NO: 122 - DNA, 30 bp, artificial
  Artificial polynucleotide
gaaagagctcgcataaggggaccagtcggt

SEQ ID NO: 123 - DNA, 20 bp, artificial
  Artificial polynucleotide
ccttcgcaagacccttcctc

SEQ ID NO: 124 - DNA, 39 bp, artificial; identical to SEQ ID NO: 60
  Artificial polynucleotide

SEQ ID NO: 125 - DNA, 39 bp, artificial; identical to SEQ ID NO: 61
  Artificial polynucleotide

SEQ ID NO: 126 - DNA, 39 bp, artificial; identical to SEQ ID NO: 60
  Artificial polynucleotide

SEQ ID NO: 127 - DNA, 39 bp, artificial; identical to SEQ ID NO: 61
  Artificial polynucleotide